(19) United States
(12) Patent Application Publication
(10) Pub. No.: US 2006/0234347 A1
(43) Pub. Date: Oct. 19, 2006

(54) Targeting multiple angiogenic pathways for cancer therapy using soluble tyrosine kinase receptors

(76) Inventors: Thomas C. Harding, San Francisco, CA (US); Minh Nguyen, San Francisco, CA (US)

Correspondence Address: DLA Piper Rudnick Gray Cary LLP, Suite 800, 153 Townsend Street, San Francisco, CA, 94107-1957, US

(21) Appl. No.: 11/401,340

(22) Filed: Apr. 10, 2006

Related U.S. Application Data
(60) Provisional application No. 60/670,639, filed on Apr. 13, 2005.

(51) Int. Cl. C12P 21/06; C07H 21/04; C07K 14/71; C12N 15/86

(52) U.S. Cl. 435/69.1; 435/320.1; 435/325; 530/350; 536/23.5; 435/456

(57) ABSTRACT

Multivalent soluble receptor proteins that bind to more than one angiogenic factor are described. Nucleotide and vector sequences which encode the multivalent soluble receptor protein, as well as host cells which comprise them and methods of making and using them are also described. The multivalent soluble receptor proteins and vectors which encode them find utility in treatment of cancer and other diseases associated with angiogenesis.

### CROSS-REFERENCES TO RELATED APPLICATIONS

This application claims the benefit of U.S. Patent Application No. 60/670,639, filed Apr. 13, 2005, the contents of which is hereby incorporated by reference in it's entirety.

### BACKGROUND OF THE INVENTION

1. Field of the Invention

The present invention relates to multivalent soluble receptor proteins that bind multiple angiogenic factors and nucleic acids which encode them. The present invention also relates to methods of inhibiting angiogenesis and methods of treating cancer using such multivalent soluble receptor constructs.

2. Background of the Technology

Angiogenesis, the development of new blood vessels from an existing vascular bed, is a complex multistep process that involves the degradation of components of the extracellular matrix and then the migration, proliferation and differentiation of endothelial cells to form tubules and eventually new vessels. Angiogenesis is important in normal physiological processes including, for example, embryo implantation; embryogenesis and development and wound healing. Excessive angiogenesis is also involved in pathological conditions such as tumour cell growth and non-cancerous conditions such as neovascular glaucoma, rheumatoid arthritis, psoriasis and diabetic retinopathy. The vascular endothelium is normally quiescent. However, upon activation, endothelial cells proliferate and migrate to form a primitive tubular network which will ultimately form a capillary bed to supply blood to developing tissues including a growing tumour.

Persistent, unregulated angiogenesis occurs in a multiplicity of disease states, tumor metastasis and abnormal growth by endothelial cells and is believed to contribute to the pathology of these conditions. The diverse pathological states created due to unregulated angiogenesis have been grouped together as angiogenic dependent or angiogenic associated diseases. Therapies directed at control of the angiogenic processes could lead to the abrogation or mitigation of these diseases.

Many growth factors, receptor tyrosine kinases, and other naturally occurring factors are involved at various determinant points of new blood vessel formation. A number of anti-angiogenic therapies are currently in development and there are clinical trials targeting the VEGF ligand/receptor family. Human VEGF exists as a glycosylated homodimer in one of five mature processed forms containing 206, 189, 165, 145 and 121 amino adds, the most prevalent being the 165 amino acid form. Vascular endothelial growth factor (VEGF) and its homologues impart activity by binding to vascular endothelial cell plasma membrane-spanning tyrosine kinase receptors which then activates signal transduction and cellular signals.

There are at least three recognized VEGF receptors: VEGFR1, VEGFR2 and VEGFR3. The VEGF family has a demonstrated role in a wide spectrum of cancers, particularly highly vascularized tumors; however, recent research has indicated that additional growth factor pathways are also involved in tumor progression. One method for VEGF ligand blockade is the use of soluble VEGF receptors such as those derived from VEGFR-1 or VEGFR-2. One method for constructing these molecules involves fusing the extracellular IgG-like domains of the VEGF receptors that are responsible for binding the VEGF ligand, to the human IgG1 heavy chain fragment with a signal sequence at the N-terminus for secretion.

Blocking VEGF from binding to its receptor has proven efficacious for some cancers by inhibiting early stages of tumor angiogenesis. However, other cancers do not respond to treatment against VEGF, particularly cancers that have more established vasculature or can express other angiogenic factors thereby using alternative pathways, for example, fibroblast growth factor (FGF), platelet-derived growth factor (PDGF) and epidermal growth factor (EGF).

VEGF-based soluble receptors appear to have potential in inhibiting angiogenesis and in treatment of cancer; however, there remains a need for more effective strategies to efficiently inhibit angiogenic pathways.

### SUMMARY OF THE INVENTION

The invention provides multivalent soluble receptor proteins which serve as antagonists of angiogenic factors, wherein the multivalent soluble receptor protein targets two or more receptors or pathways related to angiogenesis.

In particular, multivalent soluble receptor proteins are provided that inhibit pathways involving FGF, VEGF, PDGF, EGF, angiopoietins, hepatocyte growth factor (HGF), Insulin-like growth factor (IGF), Ephrins, placental growth factor, tumor growth factor alpha (TGFa), tumor growth factor beta (TGFb), tumor necrosis factor alpha (TNFa) or tumor necrosis factor beta (TNFb).

In one aspect, multivalent chimeric soluble receptor proteins are constructed to include multiple ligand-binding domains of different receptors such that they are targeted to more than one ligand.

The invention provides nucleotide sequences which encode multivalent soluble receptor proteins which include: (a) the coding sequence for at least two domains selected from the group consisting of a PDGFR-alpha Ig-like domain, a PDGFR-beta Ig-like domain, a Fibroblast Growth Factor Receptor 1 (FGFR1) Ig-like domain, a Fibroblast Growth Factor Receptor 2 (FGFR2) Ig-like domain, a Hepatocyte Growth Factor Receptor (HGFR) SEMA domain-like domain; and (b) the coding sequence for a heterologous multimerizing domain, for example an IgGFc domain.

In one embodiment, the nucleotide sequence encodes at least one PDGFR-alpha Ig-like domain or one PDGFR-beta Ig-like domain such as the sequence presented as SEQ ID NO:16 or SEQ ID NO:19, respectively, and at least one Fibroblast Growth Factor Receptor 1 (FGFR1) Ig-like domain such as the sequence presented as SEQ ID NO:22. In a related embodiment, the nucleotide sequence encodes at least one PDGFR-alpha Ig-like domain or one PDGFR-beta Ig-like domain such as the sequence presented as SEQ ID NO:16 or SEQ ID NO:19, respectively, and at least one Fibroblast Growth Factor Receptor 2 (FGFR2) Ig-like domain, such as the sequence presented as SEQ ID NO:25. In a further related embodiment, the nucleotide sequence encodes at least one PDGFR-alpha Ig-like domain or one PDGFR-beta Ig-like domain such as the sequence presented as SEQ ID NO:16 or SEQ ID NO:19, respectively, and at least one SEMA domain from Hepatocyte Growth Factor Receptor (HGFR), such as the sequence presented as SEQ ID NO:28.

In another embodiment, the nucleotide sequence encodes a Vascular Endothelial Growth Factor Receptor 1 (VEGFR1) Ig-like domain 2 and a Vascular Endothelial Growth Factor Receptor 2 (VEGFR2) Ig-like domain 3 together with at least two additional domains selected from the group consisting of a PDGFR-alpha Ig-like domain such as the sequence presented as SEQ ID NO:16, a PDGFR-beta Ig-like domain such as the sequence presented as SEQ ID NO:19, a Fibroblast Growth Factor Receptor 1 (FGFR1) Ig-like domain such as the sequence presented as SEQ ID NO:22, a Fibroblast Growth Factor Receptor 2 (FGFR2) Ig-like domain such as the sequence presented as SEQ ID NO:25, a Hepatocyte Growth Factor Receptor (HGFR) SEMA domain such as the sequence presented as SEQ ID NO:28; and the coding sequence for a multimerizing domain, for example an IgGFc domain.

The invention further provides vectors such as an adeno-associated virus (AAV) vector, a retroviral vector, a lentiviral vector, an adenovirus (Ad) vector, a simian virus 40 (SV-40) vector, a bovine papilloma virus vector, an Epstein-Barr virus vector, a herpes virus vector, and a vaccinia virus vector comprising a multivalent soluble receptor encoding nucleotide sequence and host cells comprising such vectors.

The invention further discloses methods for producing multivalent soluble receptor proteins, using the vectors and host cells described hereinabove.

The invention also provides methods of inhibiting angiogenesis and lymphangiogeneis in vivo (e.g. in a mammal) by delivering a multivalent soluble receptor protein of the invention and/or a vector expressing a multivalent soluble receptor protein to a subject.

### BRIEF DESCRIPTION OF THE FIGURES

FIG. 1 depicts multivalent soluble FGF and PDGF receptor/IgG fusion proteins: PDGF-alpha domains 1-5 linked to a dimer domain (i.e., IgGFc) (FIG. 1A); PDGF-beta domains 1-5 linked to a dimer domain (i.e., IgGFc) (FIG. 1B); FGFR1 domains 1-3 linked to a dimer domain (i.e., IgGFc) (FIG. 1C); FGFR2 domains 2-3 linked to a dimer domain (i.e., IgGFc) (FIG. 1D); VEGFR1 domain 2 and VEGFR2 domain 3 linked to a dimer domain (i.e., IgGFc) (FIG. 1E).

FIG. 2 depicts multivalent soluble receptor fusion proteins that contain ligand binding motifs for more than one factor incorporated into a single molecule wherein the molecules comprise in the N terminal to C-terminal direction: VEGFR1 domain 2 and VEGFR2 domain 3 linked to PDGF-beta domains 1-5 and a dimer domain (IgGFc) (FIG. 2A); PDGF-beta domains 1-5 linked to VEGFR1 domain 2 and VEGFR2 domain 3 and a dimer domain (IgGFc) (FIG. 2B); VEGFR1 domain 2 and VEGFR2 domain 3 linked to a dimer domain (IgGFc) and PDGF-beta domains 1-5 (FIG. 2C); PDGF-beta domains 1-5 linked to a dimer domain (IgGFc) and VEGFR1 domain 2 and VEGFR2 domain 3 (FIG. 2D); VEGFR1 domain 2 and VEGFR2 domain 3 linked to a dimer domain (IgGFc) and FGFR1 domains 1-3 (FIG. 2E); VEGFR1 domain 2 and VEGFR2 domain 3 linked to a dimer domain (IgGFc) and VEGFR3 domains 1-3 (FIG. 2F); PDGF-alpha domains 1-5 linked to a dimer domain (IgGFc) and FGFR1 domains 1-3 (FIG. 2G); VEGFR1 domain 2 and VEGFR2 domain 3 linked to a dimer domain (IgGFc) and FGFR1 domains 1-3 (FIG. 2H).

FIG. 3 depicts single AAV expression vectors for the dual production/expression of multivalent soluble receptor fusion proteins: internal ribosome entry (IRES) based construct (FIG. 3A); bi-directional promoter based construct (FIG. 3B); and protease cleavage site based construct (FIG. 3C).

FIGS. 4A and 4B show the amino acid sequence of the extracellular domain of VEGFR1 (SEQ ID NO: 50), VEGFR2 (SEQ ID NO: 49) and VEGFR3 (SEQ ID NO: 48). Each of the seven Ig-like domains for each protein are labeled.

FIG. 5 shows an annotated version of the amino acid sequence of the multivalent soluble receptor fusion proteins sVEGFR-PDGFR beta domains 1-5 IgGFc (SEQ ID NO:51).

FIG. 6 shows an annotated version of the amino acid sequence of the multivalent fusion protein sPDGFR beta domains 1-5-VEGFR-IgGFc (SEQ ID NO:52)

FIG. 7 shows an annotated version of the amino acid sequence of the multivalent fusion protein sVEGFR-IgGFc-sPDGFR beta domains 1-5 (SEQ ID NO:53).

FIG. 8 shows an annotated version of the amino acid sequence of the multivalent fusion protein sPDGFR beta domains 1-5-IgGFc-VEGFR (SEQ ID NO:54)

FIG. 9 depicts a plasmid map of pTR-CAG-VEGF-TRAP-WPRE-BGHpA (SEQ ID NO:38). This plasmid contains the following sequences: VEGF-Trap (Start: 1908 End: 3284); AAV-2 5′ ITR (Start: 7 End: 136); CAG Promoter (Start: 217 End: 1910); VEGFR1 Signal sequence (Start: 1908 End: 1981) VEGFR1 D2 (Start: 1985 End: 2287); IgG1 Fc (Start: 2605 End: 3284); WPRE (Start: 3339 End: 3929); BGHpA Signal (Start: 3952 End: 4175); AAV-2 3′ ITR (Start: 4245 End: 4372 (Complementary)).

FIG. 10 depicts a plasmid map of pTR-CAG-sPDGFRb1-5Fc (SEQ ID NO:39). This plasmid contains the following sequences: AAV-2 5′ ITR (Start: 7 End: 136); CAG promoter/introns (Start: 217 End: 1901); PDGFRb domains1-5 (Start: 1915 End: 3506); IgG1 Fc (Start: 3521 End: 4200); WPRE (Start: 4255 End: 4845); BGHpA Signal (Start: 4868 End: 5091); and AAV-2 3′ ITR (Start: 5161 End: 5288 (Complementary)).

### DETAILED DESCRIPTION OF THE INVENTION

The present invention provides multivalent soluble receptor fusion protein compositions and methods for inhibiting multiple angiogenesis pathways using multivalent soluble receptor fusion proteins. Without being bound by theory, the inventors believe that targeting and inhibiting multiple angiogenesis pathways will more effectively inhibit angiogenesis and/or lymphangiogenesis.

The present invention may be described herein as targeting and inhibiting multiple angiogenic pathways. This is accomplished utilizing either a single vector that encodes a multivalent soluble receptor fusion protein or a multivalent soluble receptor fusion protein.

The invention provides several advantages. First, the vectors and fusion proteins of the invention target more than one angiogenic pathways. Blocking only one angiogenic pathway may not completely or even significantly block the angiogenic process pathway. For example, tumors require the angiogenesis process to increase their mass or size. Methods used to block a VEGF pathway may not completely block angiogenesis and therefore the tumor can continue growing. Tumors can express more than one angiogenic factors thereby using alternative angiogenic pathways, including PDGF, FGF, HGF and EGF and the like. Blocking these pathways can facilitate more effective inhibition of angiogenesis and result in a corresponding reduction in tumor growth and tumor regression.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992, Current Protocols in Molecular Biology (John Wiley & Sons, including periodic updates); Glover, 1985, DNA Cloning (IRL Press, Oxford); Anand, 1992, Techniques for the Analysis of Complex Genomes, Academic Press, New York; Guthrie and Fink, 1991, Guide to Yeast Genetics and Molecular Biology, Academic Press, New York; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); “Current Protocols in Immunology” (J. E. Coligan et al., eds., 1991); Riott, Essential Immunology, 6th Edition, Blackwell Scientific Publications, Oxford, 1988; Hogan et al., “PCR: The Polymerase Chain Reaction”, (Mullis et al., eds., 1994); Manipulating the Mouse Embryo, (Cold Spring Harbor Laboratory Press, Cold Spring Harbor, N.Y., 1986).

Definitions

Unless otherwise indicated, all terms used herein have the same meaning as they would to one skilled in the art and the practice of the present invention will employ, conventional techniques of microbiology and recombinant DNA technology, which are within the knowledge of those of skill of the art.

As used herein, the terms “multivalent soluble receptor protein” and “multivalent soluble receptor fusion molecule” may be used interchangeably and refer to fusions between two or more receptor components factors linked to a dimerizing or multimerizing domain (such as IgGFc), wherein the multivalent soluble receptor fusion molecule targets two or more receptors or pathways related to angiogenesis.

As used herein, the term “angiogenic factor” refers to a protein that stimulates angiogenesis. Exemplary angiogenic factors include, but are not limited to, VEGF proteins, FGF proteins, PDGF proteins, HGF proteins, EGF proteins and IGF proteins, angiopoietins (e.g. angiopoietin-1 (Ang-1), angiopoietin-2 (Ang-2)), Ephrin ligands (e.g. Ephrin B2, A1, A2), Integrin AV, Integrin B3, placental growth factor (PLGF), tumor growth factor-alpha (TGF-a), tumor growth factor-beta (TGF-b), tumor necrosis factor-alpha (TNF-a) and tumor necrosis factor-beta (TNF-b).

As used herein, “VEGF” refers to vascular endothelial growth factor. There are several forms of VEGF including, but not limited to, VEGF-206, VEGF-189, VEGF-165, VEGF-145, VEGF-121, VEGF-A, VEGF-B, VEGF-C and VEGF-D.

As used herein, “homologue of VEGF” refers to homodimers of VEGF-B, VEGF-C, VEGF-D and PIGF and any functional heterodimers formed between VEGF-A, VEGF-B, VEGF-C, VEGF-D and PIGF, including but not limited to a VEGF-A/PIGF heterodimer.

As used herein, “KDR” or “FLK-1” or “VEGFR2” refer to a kinase insert domain-containing receptor or fetal liver kinase or vascular endothelial growth factor receptor 2.

As used herein, “FLT-1” or “VEGFR1 ” refers to a fms-like tyrosine kinase receptor, also known as vascular endothelial growth factor receptor 1.

As used herein, the term “PDGFR” includes all receptors for PDGF including PDGFR-alpha and PDGFR-beta.

As used herein, the term “FGFR” includes all receptors for FGF including FGFR1 and FGFR2.

As used herein, the term “ligand” refers to a molecule capable of being bound by the ligand-binding domain of a receptor or a receptor analog. The “ligand” may be synthetic or may occur in nature. Ligands are typically grouped as agonists (a ligand wherein binding to a receptor induces the response pathway within a cell) and antagonists (a ligand wherein binding to a receptor blocks the response pathway within a cell).

As used herein, the “ligand-binding domain” of a receptor is that portion of the receptor that is involved with binding the natural ligand.

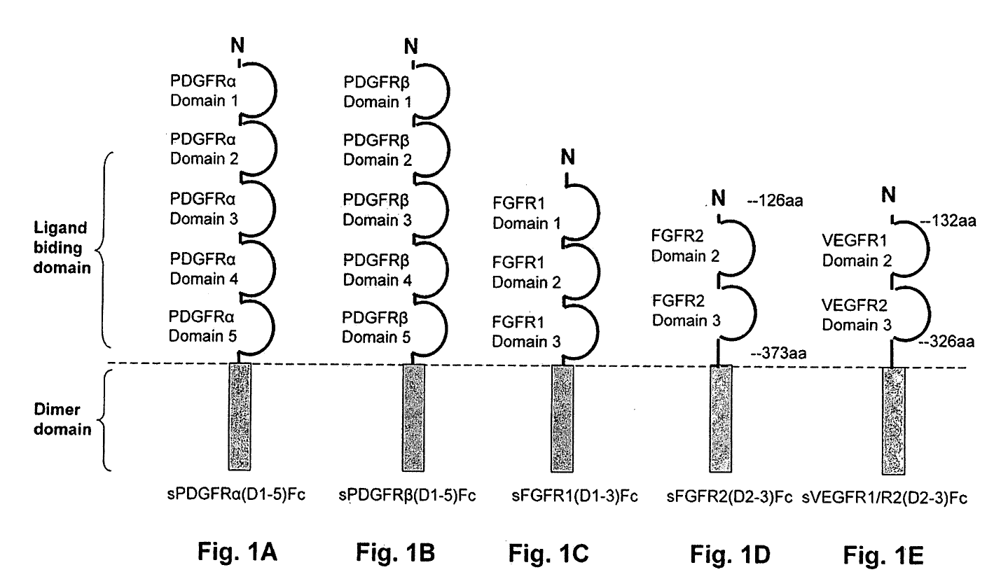

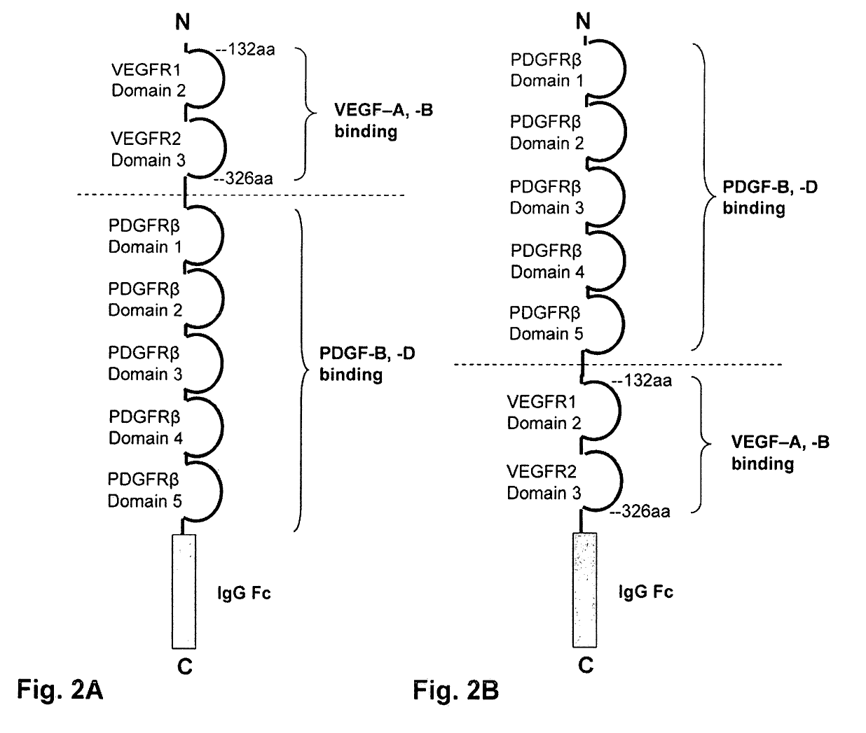

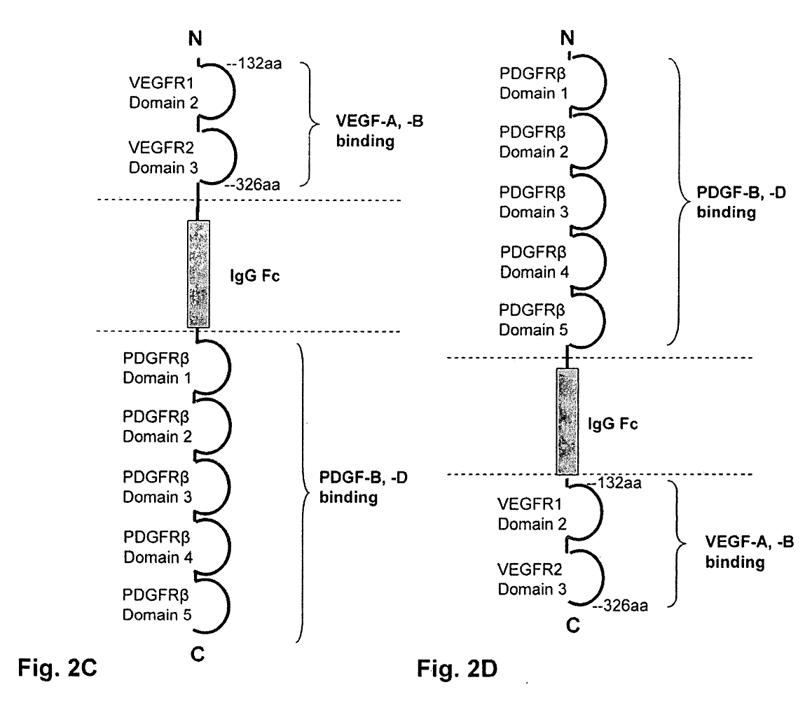

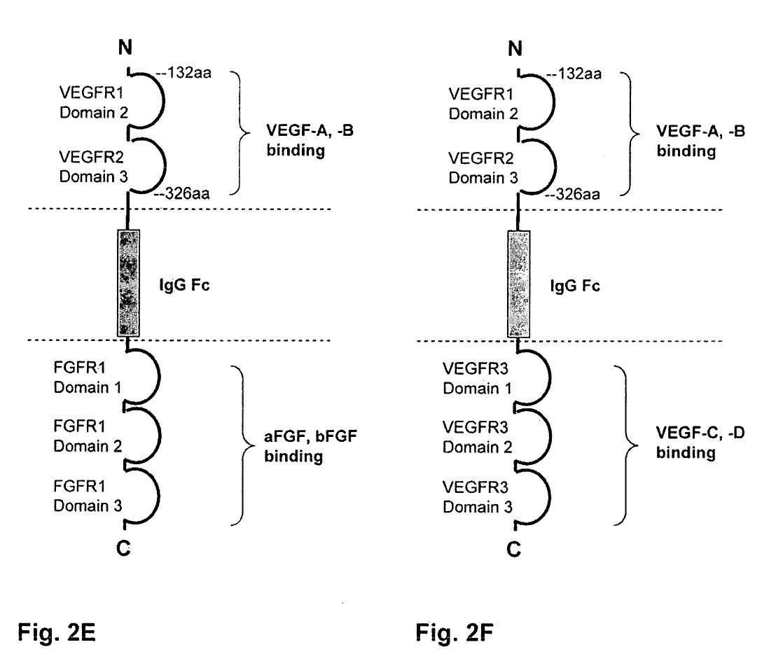

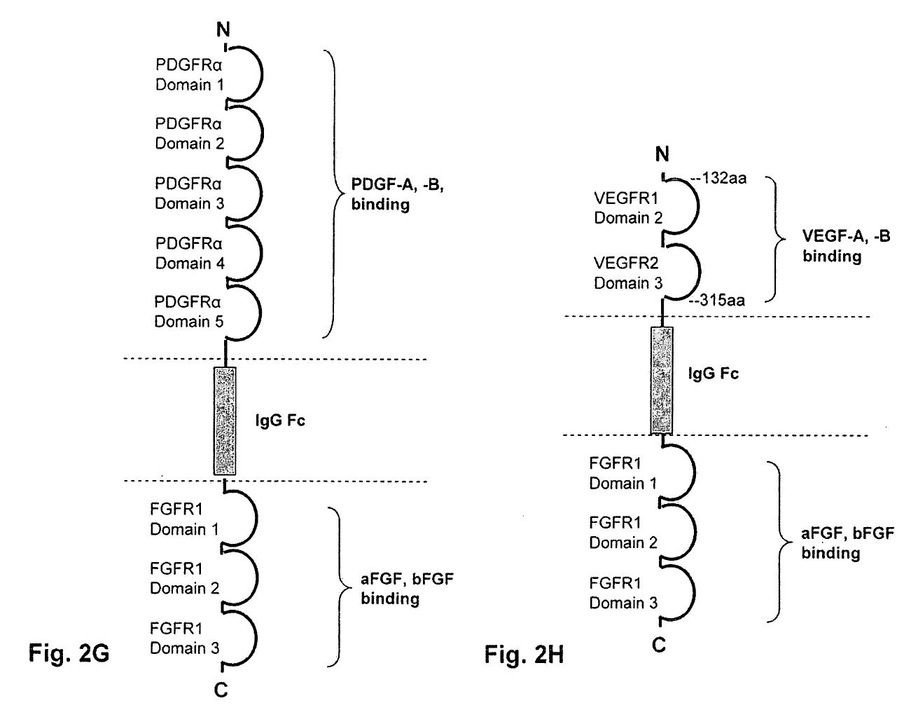

As used herein, the term “immunoglobulin domain” or “Ig-like domain” refers to each of the independent and distinct domains that are found in the extracellular ligand region of a multivalent soluble receptor proteins of the invention. The “immunoglobulin-like domain” or “Ig-like domain” refers to each of the seven independent and distinct domains that are found in the extracellular ligand-binding region of the fit-1, KDR and FLT4 receptors. Ig-like domains are generally referred to by number, the number designating the specific domain as it is shown in FIGS. 1 and 2. As used herein, the term “Ig-like domain” is intended to encompass not only the complete wild-type domain, but also insertional, deletional and substitutional variants thereof which substantially retain the functional characteristics of the intact domain. It will be readily apparent to those of ordinary skill in the art that numerous variants of Ig-like domains can be obtained which retain substantially the same functional characteristics as the wild type domain.

The term “multimerizing domain” or “multimerizing component” as used herein refers to a domain, such as the Fc domain from an IgG that is heterologous to the binding domains of a multivalent soluble receptor protein of the invention. A multimerizing domain may be essentially any polypeptide that forms a dimer (or higher order complex, such as a trimer, tetramer, etc.) with another polypeptide. Optionally, the multimerizing domain associates with other, identical multimerizing domains, thereby forming homomultimers. An IgG Fc element is an example of a dimerizing domain that tends to form homomultimers. As used herein the term multimerizing domain may be used to refer to a dimerizing, trimerinzing, tertramerizing domain, etc. In a preferred embodiment, the Ig-like domain of interest is fused to the N-terminus of the Fc domain of immunoglobulin G1 (IgG1). In some cases, the entire heavy chain constant region is fused to the VEGF receptor Ig-like domains of interest. However, more preferably, a sequence beginning in the hinge region just upstream of the papain cleavage site which defines Fc chemically, or analogous sites of other immunoglobulins are used in the fusion.

The term “extracellular ligand binding domain” is defined as the portion of a receptor that, in its native conformation in the cell membrane, is oriented extracellularly where it can contact with its cognate ligand. The extracellular ligand binding domain does not include the hydrophobic amino acids associated with the receptor's transmembrane domain or any amino acids associated with the receptor's intracellular domain. Generally, the intracellular or cytoplasmic domain of a receptor is usually composed of positively charged or polar amino acids (i.e. lysine, arginine, histidine, glutamic acid, aspartic acid). The preceding 15-30, predominantly hydrophobic or apolar amino acids (i.e. leucine, valine, isoleucine, and phenylalanine) comprise the transmembrane domain. The extracellular domain comprises the amino acids that precede the hydrophobic transmembrane stretch of amino acids. Usually the transmembrane domain is flanked by positively charged or polar amino acids such as lysine or arginine. (See von Heijne, 1995, BioEssays 17: 25-30.)

The term “soluble” as used herein with reference to the multivalent soluble receptor proteins of the present invention is intended to mean chimeric proteins which are not fixed to the surface of cells via a transmembrane domain. As such, soluble forms of the multivalent soluble receptor proteins of the present invention, while capable of binding to and inactivating VEGF, do not comprise a transmembrane domain and thus generally do not become associated with the cell membrane of cells in which the molecule is expressed.

The term “membrane-bound” as used herein with reference to the multivalent soluble receptor proteins of the present invention is intended to mean chimeric proteins which are fixed, via a transmembrane domain, to the surface of cells in which they are expressed.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring DNA into cells either in vitro or in vivo. For purposes of the present invention, these terms refer to adenoviruses, including recombinant adenoviruses formed when an adenoviral vector of the invention is encapsulated in an adenovirus capsid.

An “adenovirus vector” or “adenoviral vector” (used interchangeably) as referred to herein is a polynucleotide construct, which is replication competent or replication incompetent (e.g. defective).

Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein. Hence, the terms “adenovirus vector” or “adenoviral vector” as used herein include adenovirus or adenoviral particles.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. Preferably, a vector of the invention comprises DNA. As used herein, “DNA” includes not only bases A, T, C, and G, but also includes any of their analogs or modified forms of these bases, such as methylated nucleotides, interncleotide modifications such as uncharged linkages and thioates, use of sugar analogs, and modified and/or alternative backbone structures, such as polyamides.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleotide sequence probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The terms “coding sequence” and “coding region” refer to a nucleotide sequence that is transcribed into RNA such as mRNA, rRNA, tRNA, snRNA, sense RNA or antisense RNA. In one embodiment, the RNA is then translated in a cell to produce a protein.

The term “ORF” means open reading frame.

The term “gene” refers to a defined region that is located within a genome and that, in addition to the aforementioned coding sequence, comprises other, primarily regulatory, nucleotide sequences responsible for the control of expression, i.e., transcription and translation of the coding portion. A gene may also comprise other 5′ and 3′ untranslated sequences and termination sequences. Depending on the source of the gene, further elements that may be present are, for example, introns.

The terms “heterologous” and “exogenous” as used herein with reference to nucleotide sequences such as promoters and gene coding sequences, refer to sequences that originate from a source foreign to a particular virus or host cell or, if from the same source, are modified from their original form. Thus, a heterologous gene in a virus or cell includes a gene that is endogenous to the particular virus or cell but has been modified through, for example, codon optimization. The terms also include non-naturally occurring multiple copies of a naturally occurring nucleotide sequences. Thus, the terms refer to a nucleotide sequence that is foreign or heterologous to the virus or cell, or homologous to the virus or cell but in a position within the host viral or cellular genome in which it is not ordinarily found.

The terms “complement” and “complementary” refer to two nucleotide sequences that comprise antiparallel nucleotide sequences capable of pairing with one another upon formation of hydrogen bonds between the complementary base residues in the antiparallel nucleotide sequences.

The term “native” refers to a gene that is present in the genome of the wildtype virus or cell.

The term “naturally occurring” or “wildtype” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

The term “recombinant” as used herein with reference to nucleotide sequences refers to a combination of nucleotide sequences that are joined together using recombinant DNA technology into a progeny nucleotide sequence. As used herein with reference to viruses, cells, and organisms, the terms “recombinant,” “transformed,” and “transgenic” refer to a host virus, cell, or organism into which a heterologous nucleotide sequence has been introduced. The nucleotide sequence can be stably integrated into the genome of the host or the nucleotide sequence can also be present as an extrachromosomal molecule. Such an extrachromosomal molecule can be auto-replicating. Recombinant viruses, cells, and organisms are understood to encompass not only the end product of a transformation process, but also recombinant progeny thereof. A “non-transformed,” “non-transgenic,” or “non-recombinant” host refers to a wildtype virus, cell, or organism that does not contain the heterologous nucleotide sequence.

“Regulatory elements” are sequences involved in controlling the expression of a nucleotide sequence. Regulatory elements include promoters, enhancers, and termination signals. They also typically encompass sequences required for proper translation of the nucleotide sequence.

The term “promoter” refers to an untranslated DNA sequence usually located upstream of the coding region that contains the binding site for RNA polymerase II and initiates transcription of the DNA. The promoter region may also include other elements that act as regulators of gene expression. The term “minimal promoter” refers to a promoter element, particularly a TATA element that is inactive or has greatly reduced promoter activity in the absence of upstream activation elements.

As used herein, a “regulatable promoter” is any promoter whose activity is affected by a cis or trans acting factor (e.g., an inducible promoter, such as an external signal or agent).

As used herein, a “constitutive promoter” is any promoter that directs RNA production in many or all tissue/cell types at most times, e.g., the human CMV immediate early enhancer/promoter region which promotes constitutive expression of cloned DNA inserts in mammalian cells.

The term “enhancer” within the meaning of the invention may be any genetic element, e.g., a nucleotide sequence that increases transcription of a coding sequence operatively linked to a promoter to an extent greater than the transcription activation effected by the promoter itself when operatively linked to the coding sequence, i.e. it increases transcription from the promoter.

The terms “transcriptional regulation elements” and “translational regulation elements” are those elements that affect transcription and/or translation of nucleotide sequences. These elements include, but are not limited to, splice donor and acceptor sites, translation stop and start codons, and adenylation signals.

As used herein, a “transcriptional response element” or “transcriptional regulatory element”, or “TRE” is a polynucleotide sequence, preferably a DNA sequence, comprising one or more enhancer(s) and/or promoter(s) and/or promoter elements such as a transcriptional regulatory protein response sequence or sequences, which increases transcription of an operatively linked polynucleotide in a host cell that allows a TRE to function.

“Under transcriptional control” is a term well understood in the art and indicates that transcription of a polynucleotide sequence, usually a DNA sequence, depends on its being operatively linked to an element which contributes to the initiation of, or promotes, transcription.

The term “operatively linked” relates to the orientation of polynucleotide elements in a functional relationship. An IRES is operatively linked to a coding sequence if the IRES promotes transcription of the coding sequence. Operatively linked means that the DNA sequences being linked are generally contiguous and, where necessary to join two protein coding regions, contiguous and in the same reading frame. However, since enhancers generally function when separated from the promoter by several kilobases and intronic sequences may be of variable length, some polynucleotide elements may be operatively linked but not contiguous.

As used herein, “co-transcribed” means that two (or more) coding regions or polynucleotides are under transcriptional control of a single transcriptional control or regulatory element.

The term “vector”, as used herein, refers to a nucleotide sequence or construct designed for transfer between different host cells. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector. Any vector for use in gene introduction can basically be used as a “vector” into which the DNA having the desired sequence is to be introduced. Plasmid vectors will find use in practicing the present invention. The term vector as it applies to the present invention is used to describe a recombinant vector, e.g., a plasmid or viral vector (including a replication defective or replication competent virus). The terms “vector,” “polynucleotide vector,” “polynucleotide vector construct,” “nucleotide sequence vector construct,” and “vector construct” are used interchangeably herein to mean any construct for gene transfer, as understood by one skilled in the art.

The term “coding region”, as used herein, refers to a nucleotide sequence that contains the coding sequence. The coding region may contain other regions from the corresponding gene including introns. The term “coding sequence” (CDS) refers to the nucleotide sequence containing the codons that encode a protein. The coding sequence generally begins with a translation start codon (e.g. ATG) and ends with a translation stop codon. Sequences said to be upstream of a coding sequence are 5′ to the translational start codon and sequences downstream of a CDS are 3′ of the translational stop codon.

The term “homologous” as used herein with reference to nucleotide molecule refers to a nucleotide sequence naturally associated with a host virus or cell.

The terms “identical” or percent “identity” are used herein in the context of two or more nucleotide sequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

As used herein, the term “sequence identity” refers to the degree of identify between nucleotides in two or more aligned sequences, when aligned using a sequence alignment program. The term “% homology” is used interchangeably herein with the term “% identity” herein and refers to the level of nucleotide or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence.

“Transformation” is typically used to refer to bacteria comprising heterologous DNA or cells which express an oncogene and have therefore been converted into a continuous growth mode such as tumor cells. A vector used to “transform” a cell may be a plasmid, virus or other vehicle.

Typically, a cell is referred to as “transduced”, “infected”, “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

As used herein, the terms “stably transformed”, “stably transfected” and “transgenic” refer to cells that have a non-native (heterologous) nucleotide sequence integrated into the genome. Stable transfection is demonstrated by the establishment of cell lines or clones comprised of a population of daughter cells containing the transfected DNA stably integrated into their genomes. In some cases, “transfection” is not stable, i.e., it is transient. In the case of transient transfection, the exogenous or heterologous DNA is expressed, however, the introduced sequence is not integrated into the genome and is considered to be episomal.

The terms “administering” or “introducing”, as used herein refer to delivery of a vector for recombinant protein expression to a cell or to cells and or organs of a subject. Such administering or introducing may take place in vivo, in vitro or ex vivo. A vector for recombinant protein or polypeptide expression may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

As used herein, “ex vivo administration” refers to a process where primary cells are taken from a subject, a vector is administered to the cells to produce transduced, infected or transfected recombinant cells and the recombinant cells are readministered to the same or a different subject.

The term “replication defective” as used herein relative to a viral vector of the invention means the viral vector cannot further replicate and package its genomes. For example, when the cell of a subject are infected with an adenoviral vector that has the entire E1 and the E4 coding region deleted or inactivated, the heterologous transgene is expressed in the patient's cells if the transgene is transcriptionally active in the cell. However, due to the fact that the patient's cells lack the Ad E1 and E4 coding sequences, the Ad vector is replication defective and viral particles cannot be formed in these cells

The term “replication competent” means the vector can replicate in particular cell types (“target cells”), e.g., cancer cells and preferentially effect cytolysis of those cells. Specific replication competent viral vectors have been developed for which selective replication in cancer cells preferentially destroys those cells. Various cell-specific replication competent adenovirus constructs, which preferentially replicate in (and thus destroy) certain cell types. Such viral vectors may be referred to as “oncolytic viruses” or “oncolytic vectors” and may be considered to be “cytolytic” or “cytopathic” and to effect “selective cytolysis” of target cells. Examples of “replication competent” or “oncolytic” viral vectors are described in, for example PCT Publication Nos. WO98/39466, WO95/19434, WO97/01358, WO98/39467, WO98/39465, WO01/72994, WO 04/009790, WO 00/15820, WO 98/14593, WO 00/46355, WO 02/067861, WO 98/39464, WO 98/13508, WO 20004/009790; U.S. Provisional Application Ser. Nos. 60/511,812, 60/423,203 and US Patent Publication No. US20010053352, expressly incorporated by reference herein.

The terms “replication conditional viruses”, “preferentially replicating viruses”, “specifically replicating viruses” and “selectively replicating viruses” are terms that are used interchangeably and are replication competent viral vectors and particles that preferentially replicate in certain types of cells or tissues but to a lesser degree or not at all in other types. In one embodiment of the invention, the viral vector and/or particle selectively replicates in tumor cells and or abnormally proliferating tissue, such as solid tumors and other neoplasms. Such viruses may be referred to as “oncolytic viruses” or “oncolytic vectors” and may be considered to be “cytolytic” or “cytopathic” and to effect “selective cytolysis” of target cells.

The term “plasmid” as used herein refers to a DNA molecule that is capable of autonomous replication within a host cell, either extrachromosomally or as part of the host cell chromosome(s). The starting plasmids herein are commercially available, are publicly available on an unrestricted basis, or can be constructed from such available plasmids as disclosed herein and/or in accordance with published procedures. In certain instances, as will be apparent to the ordinarily skilled artisan, other plasmids known in the art may be used interchangeable with plasmids described herein.

The term “expression” refers to the transcription and/or translation of an endogenous gene, transgene or coding region in a cell.

A “polyadenylation signal sequence” is a recognition region for endonuclease cleavage of a RNA transcript that is followed by a polyadenylation consensus sequence AATAAA. A polyadenylation signal sequence provides a “polyA site”, i.e. a site on a RNA transcript to which adenine residues will be added by post-transcriptional polyadenylation. Generally, a polyadenylation signal sequence includes a core poly(A) signal that consists of two recognition elements flanking a cleavage-polyadenylation site (e.g., FIG. 1 of WO 02/067861 and WO 02/068627). The choice of a suitable polyadenylation signal sequence will consider the strength of the polyadenylation signal sequence, as completion of polyadenylation process correlates with poly(A) site strength (Chao et al., Molecular and Cellular Biology, 1999, 19:5588-5600). For example, the strong SV40 late poly(A) site is committed to cleavage more rapidly than the weaker SV40 early poly(A) site. The person skilled in the art will consider choosing a stronger polyadenylation signal sequence if desired. In principle, any polyadenylation signal sequence may be useful for the purposes of the present invention. However, in some embodiments of this invention the termination signal sequence is the SV40 late polyadenylation signal sequence or the SV40 early polyadenylation signal sequence. Usually, the termination signal sequence is isolated from its genetic source or synthetically constructed and inserted into a vector of the invention at a suitable position.

A “multicistronic transcript” refers to a mRNA molecule that contains more than one protein coding region, or cistron. A mRNA comprising two coding regions is denoted a “bicistronic transcript.” The “5′-proximal” coding region or cistron is the coding region whose translation initiation codon (usually AUG) is closest to the 5′-end of a multicistronic mRNA molecule. A “5′-distal” coding region or cistron is one whose translation initiation codon (usually AUG) is not the closest initiation codon to the 5′ end of the mRNA. The terms “5′-distal” and “downstream” are used synonymously to refer to coding regions that are not adjacent to the 5′ end of a mRNA molecule.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene (Jackson R J, Howell M T, Kaminski A (1990) Trends Biochem Sci 15(12):477-83) and Jackson R J and Kaminski, A. (1995) RNA 1(10):985-1000). The present invention encompasses the use of any IRES element, which is able to promote direct internal ribosome entry to the initiation codon of a cistron. PCT publication WO 01/55369 describes examples of IRES sequences including synthetic sequences and these sequences may also be used according to the present invention. “Under translational control of an IRES” as used herein means that translation is associated with the IRES and proceeds in a cap-independent manner. Examples of “IRES” known in the art include, but are not limited, to IRES obtainable from picornavirus (Jackson et al., 1990, Trends Biochem Sci 15(12):477-483); and IRES obtainable from viral or cellular mRNA sources, such as for example, immunoglobulin heavy-chain binding protein (BiP), the vascular endothelial growth factor (VEGF) (Huez et al. (1998) Mol. Cell. Biol. 18(11):6178-6190), the fibroblast growth factor 2, and insulin-like growth factor, the translational initiation factor eIF4G, yeast transcription factors TFIID and HAP4. IRES have also been reported in different viruses such as cardiovirus, rhinovirus, aphthovirus, HCV, Friend murine leukemia virus (FrMLV) and Moloney murine leukemia virus (MoMLV). As used herein, “IRES” encompasses functional variations of IRES sequences as long as the variation is able to promote direct internal ribosome entry to the initiation codon of a cistron. In some embodiments, the IRES is mammalian. In other embodiments, the IRES is viral or protozoan. In one embodiment, the IRES is obtainable from encephelomycarditis virus (ECMV) (commercially available from Novogen, Duke et al. (1992) J. Virol 66(3):1602-1609). In another illustrative embodiment disclosed herein, the IRES is from VEGF. Examples of IRES sequences are described in U.S. Pat. No. 6,692,736.

A “self-processing cleavage site” or “self-processing cleavage sequence” as referred to herein is a DNA or amino acid sequence, wherein upon translation, rapid intramolecular (cis) cleavage of a polypeptide comprising the self-processing cleavage site occurs to result in expression of discrete mature protein or polypeptide products. Such a “self-processing cleavage site”, may also be referred to as a post-translational or co-translational processing cleavage site, e.g., a 2A site, sequence or domain. A 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site, sequence or domain demonstrates “auto-proteolysis” or “cleavage” by cleaving its own C-terminus in cis to produce primary cleavage products (Furler; Palmenberg, Ann. Rev. Microbiol. 44:603-623 (1990)).

A “self-processing cleavage site” or “self-processing cleavage sequence” is defined herein as a post-translational or co-translational processing cleavage site or sequence. Such a “self-processing cleavage” site or sequence refers to a DNA or amino acid sequence, exemplified herein by a 2A site, sequence or domain or a 2A-like site, sequence or domain. As used herein, a “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

As used herein, the term “additional proteolytic cleavage site”, refers to a sequence which is incorporated into an expression construct of the invention adjacent a self-processing cleavage site, such as a 2A or 2A like sequence, and provides a means to remove additional amino acids that remain following cleavage by the self processing cleavage sequence. Exemplary “additional proteolytic cleavage sites” are described herein and include, but are not limited to, furin cleavage sites with the consensus sequence RXK(R)R (SEQ ID NO: 44). Such furin cleavage sites can be cleaved by endogenous subtilisin-like proteases, such as furin and other serine proteases within the protein secretion pathway.

In one embodiment, the invention provides a method for removal of residual amino acids and a composition for expression of the same. A number of novel constructs have been designed that provide for removal of these additional amino acids from the C-terminus of the protein. Furin cleavage occurs at the C-terminus of the cleavage site, which has the consensus sequence RXR(K)R (SEQ ID NO: 45), where X is any amino acid. In one aspect, the invention provides a means for removal of the newly exposed basic amino acid residues R or K from the C-terminus of the protein by use of an enzyme selected from a group of enzymes called carboxypeptidases (CPs), which include, but not limited to, carboxypeptidase D, E and H(CPD, CPE, CPH), as further described in U.S. Application Ser. No. 60/659,871.

As used herein, “transgene” refers to a polynucleotide that can be expressed, via recombinant techniques, in a non-native environment or heterologous cell under appropriate conditions. In the present invention, the transgene coding region is inserted in a viral vector. In one embodiment, the viral vector is an adenoviral vector. The transgene may be derived from the same type of cell in which it is to be expressed, but introduced from an exogenous source, modified as compared to a corresponding native form and/or expressed from a non-native site, or it may be derived from a heterologous cell. “Transgene” is synonymous with “exogenous gene”, “foreign gene”, “heterologous coding sequence” and “heterologous gene”. In the context of a vector for use in practicing the present invention, a “heterologous polynucleotide” or “heterologous gene” or “transgene” is any polynucleotide or gene that is not present in the corresponding wild-type vector or virus. The transgene coding sequence may be a sequence found in nature that codes for a certain protein. The transgene coding sequence may alternatively be a non-natural coding sequence. For example, one skilled in the art can readily recode a coding sequence to optimize the codons for expression in a certain species using a codon usage chart. In one embodiment, the recoded sequence still codes for the same amino acid sequence as a natural coding sequence for the transgene. Examples of preferred transgenes for inclusion in the vectors of the invention, are provided herein. A transgene may be a therapeutic gene. A transgene does not necessarily code for a protein.

As used herein, a “therapeutic” gene refers to a transgene that, when expressed, confers a beneficial effect on a cell, tissue or mammal in which the gene is expressed. Examples of beneficial effects include amelioration of a sign or symptom of a condition or disease, prevention or inhibition of a condition or disease, or conferral of a desired characteristic. Numerous examples of therapeutic genes are known in the art, a number of which are further described below.

In the context of a vector for use in practicing the present invention, a “heterologous” sequence or element is one which is not associated with or derived from the corresponding wild-type vector or virus.

In the context of a vector for use in practicing the present invention, an “endogenous” sequence or element is native to or derived from the corresponding wild-type vector or virus.

“Replication” and “propagation” are used interchangeably and refer to the ability of a viral vector of the invention to reproduce or proliferate. These terms are well understood in the art. For purposes of this invention, replication involves production of virus proteins and is generally directed to reproduction of virus. Replication can be measured using assays standard in the art and described herein, such as a virus yield assay, burst assay or plaque assay. “Replication” and “propagation” include any activity directly or indirectly involved in the process of virus manufacture, including, but not limited to, viral gene expression; production of viral proteins, replication of nucleotides or other components; packaging of viral components into complete viruses and cell lysis.

“Preferential replication” and “selective replication” and “specific replication” may be used interchangeably and mean that the virus replicates more in a target cell than in a non-target cell. The virus replicates at a higher rate in target cells than non target cells, e.g. at least about 3-fold higher, at least about 10-fold higher, at least about 50-fold higher, and in some instances at least about 100-fold, 400-fold, 500-fold, 1000-fold or even 1×10⁶ higher. In one embodiment, the virus replicates only in the target cells (that is, does not replicate at all or replicates at a very low level in non-target cells).

As used herein, a “packaging cell” is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by the vector or by the packaging cell.

“Producer cells” for viral vectors are well known in the art. A producer cell is a cell in which the adenoviral vector is delivered and the adenoviral vector is replicated and packaged into virions. If the viral vector has an essential gene deleted or inactivated, then the producer cell complements for the inactivated gene. Examples of adenoviral vector producer cells are PerC.6 (Falluax et al. Hum Gene Ther. 1998 Sep. 1; 9(13):1909-17) and 293 cells (Graham et al. J Gen Virol. 1977 July; 36(1):59-74). In the case of selectively replicating viruses, producer cells may be of a cell type in which the virus selectively replicates. Alternatively or in addition, the producer cell may express the genes that are selectively controlled or inactivated in the viral vector.

The term “HeLa-S3” means the human cervical tumor-derived cell line available from American Type Culture Collection (ATCC, Manassas, Va.) and designated as ATCC number CCL-2.2. HeLa-S3 is a clonal derivative of the parent HeLa line (ATCC CCL-2). HeLa-S3 was cloned in 1955 by T. T. Puck et al. (J. Exp. Med. 103: 273-284 (1956)).

An “individual” is a vertebrate, a mammal, or a human. Mammals include, but are not limited to, farm animals, sport animals, rodents, primates, and pets.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

As used herein, “cytotoxicity” is a term well understood in the art and refers to a state in which a cell's usual biochemical or biological activities are compromised (i.e., inhibited). These activities include, but are not limited to, metabolism; cellular replication; DNA replication; transcription; translation; uptake of molecules. “Cytotoxicity” includes cell death and/or cytolysis. Assays are known in the art which indicate cytotoxicity, such as dye exclusion, ³H-thymidine uptake, and plaque assays.

As used herein, the terms “biological activity” and “biologically active”, refer to the activity attributed to a particular protein in a cell line in culture or in vivo. The “biological activity” of an “immunoglobulin”, “antibody” or fragment thereof refers to the ability to bind an antigenic determinant and thereby facilitate immunological function.

As used herein, the term “therapeutically effective amount” of a vector or chimeric multivalent soluble receptor protein of the present invention is an amount that is effective to either prevent, lessen the worsening of, alleviate, or cure the treated condition, in particular that amount which is sufficient to reduce or inhibit the proliferation of vascular endothelium in vivo.

As used herein, the terms “neoplastic cells”, “neoplasia”, “tumor”, “tumor cells”, “carcinoma”, “carcinoma cells”, “cancer” and “cancer cells”, (used interchangeably) refer to cells which exhibit relatively autonomous growth, so that they exhibit an aberrant growth phenotype characterized by a significant loss of control of cell proliferation. Neoplastic cells can be malignant or benign.

Multivalent Soluble Receptor Proteins

A number of anti-angiogenic therapies are currently in development (Marx, Science. 2003 Jul. 25; 301(5632):452-4). These therapies generally rely on blockage of VEGF receptors, however, recent research has indicated that additional growth factor pathways are also involved in tumor progression (Rich and Bigner, Nat Rev Drug Discov. May; 3(5):430-46 (2004); Garcia-Echeverria and Fabbro, Mini Rev Med Chem. March; 4(3):273-83 (2004)). Of these factors the tyrosine kinase receptor family members fibroblast growth factor (FGF; Powers et al. Endocr Relat Cancer. 2000 September; 7(3): 165-97), platelet derived growth factor (PDGF; Saharinen et al. J Clin Invest. 2003 May; 111(9):1277-80; Ostman Cytokine & Growth Factor Reviews 15 (2004) 275-286), epidermal growth factor (EGF), hepatocyte growth factor (HGF; Trusolino L, Comoglio P M., Nat Rev Cancer. 2002 April; 2(4):289-300) and Insulin-like growth factor (IGF) have been implicated. For a review of angiogenic factors see Harrigan, Neurosurgery 53(3) 2003 pgs 639-658.

Blocking ligands such as FGF, PDGF, EGF, angiopoietins (e.g. angiopoietin-1, angiopoietin-2), Ephrin ligands (e.g. Ephrin B2, A1, A2), IntegrinAV, Integrin B3, placental growth factor, tumor growth factor-alpha, tumor growth factor-beta, tumor necrosis factor-alpha and tumor necrosis factor-beta from binding to their receptors either alone or in addition to VEGF may lead to tumor stabilization or regression in cancer types that are unresponsive or not completely responsive to VEGF treatment alone.

Effective soluble receptors have also been identified for blocking PDGF and FGF ligand action. Tyrosine kinase receptor/IgG fusions have been described for VEGF, PDGF and FGF. Several groups have used these soluble receptors to block PDGF, FGF and VEGF binding to its respective ligand receptor to treat tumor growth in various animal models as a monotherapy (Strawn et al. 1994 J Biol Chem. August 19; 269(33):21215-22) and in combination (Ogawa et al. 2002 Cancer Gene Ther. August; 9(8):633-40). In each case one soluble receptor is delivered as either a monotherapy or is expressed individually using a viral construct. The invention provides multivalent soluble receptor proteins, vectors encoding them and methods of use. Exemplary, multivalent soluble receptor proteins are depicted in FIGS. 1A-E and FIGS. 2A-H.

The multivalent soluble receptor proteins of the invention bind to more than one angiogenic factor. In one aspect, the angiogenic factors are selected from the group consisting of FGF, PDGF, EGF, HGF, angiopoietins, IGF and VEGF. In one embodiment, the invention provides multivalent soluble receptor proteins comprising at least two Ig-like binding domains that bind angiogenic factors wherein the at least two Ig-like domains are from the extracellular portion of two different receptor proteins. The receptor proteins may be, but are not limited to, VEGFR1, VEGFR2, VEGFR3, PDGFR (e.g. PDGFR-alpha and PDGFR-beta), Tie-2 and FGFR (e.g. FGFR1 and FGFR2).

In one embodiment the binding domain binds angiogenic factors selected from the group consisting FGF, PDGF, EGF, HGF, angiopoietins, IGF and VEGF. In some embodiments, the binding domains may be comprised of one or more Ig-like domains from the extracellular portion of a receptor that binds an angiogenic factor (e.g. VEGF trap). If multiple Ig-like domains are used, they may bind to the same angiogenic factor(s) or different factors. Various domains that bind to angiogenic factors are known in the art including those domains derived from VEGFR1 (Flt1) and VEGFR2 (KDR; see WO98/13071: U.S. Pat. No. 5,712,380; U.S. Pat. No. 6,383,486; WO 97/44453; WO97/13787; WO00/7531), FGF receptor (FGFR; see U.S. Pat. No. 6,350,593; U.S. Pat. No. 6,656,728; Chellaiah et al Journal of Biological Chemistry 1999 December 274(49): 34785-34794; Powers et al Endocrine-Related Cancer 2000 7:165-197; Ogawa et al. (2002) Cancer Gene Ther. August; 9(8):633-40; Compagni et al. Cancer Res. 2000 Dec. 15; 60(24):7163-9), PDGF receptor α and_(Mahadevan et al Journal of Biological Chemistry 1995 November 270(46):27595-27600; Lokker et al. Journal of Biological Chemistry 1997 December 272(52):33037-33044; Miyazawa et al. Journal of Biological Chemistry 1998 September 273(39):25495-25502) VEGFR3 (Makiners et al Nature Medicine 2001 Feb. 7(2):199-205) and Tie2 (Lin P et al 1998 PNAS USA 95(15):8829-34).

FIGS. 2A-H depict examples of multivalent soluble receptor proteins of the invention. The multivalent soluble receptor protein may also contain a multimerizing domain, such as a Fc domain from an IgG. The Ig-like domains may be upstream (toward amino terminus), downstream (toward carboxyl terminus) or both upstream and downstream of the multimerizing domain. In one embodiment, all of the Ig-like domains of the invention are located downstream of the multimerizing domain.

In one embodiment, the multimerizing domain is a Fc domain of an IgG. For example the Fc region may be comprised of a sequence beginning in the hinge region just upstream of the papain cleavage site which defines Fc chemically, or analogous sites of other immunoglobulins. In some embodiments, the encoded chimeric polypeptide retains at least functionally active hinge, CH2 and CH3 domains of the constant region of an immunoglobulin heavy chain. In some embodiments, fusions are also made to the C-terminus of the Fc portion of a constant domain, or immediately N-terminal to the CH1 of the heavy chain or the corresponding region of the light chain. In one preferred embodiment, the Ig-like domain of interest is fused to the N-terminus of the Fc domain of immunoglobulin G₁ (IgG-1).

The ligand-binding domains of a soluble chimeric receptor protein of the invention may or may not be linked by a linking sequence such as a peptide linker. The linking sequence is used to covalently connect two or more individual domains linked of the soluble chimeric receptor protein and is located between the 2 domains. Preferably, the linker increases flexibility of the binding domains and does not to interfere significantly with the structure of each functional binding domain within the soluble chimeric receptor protein. The peptide linker L is preferably between 2-50 amino acids in length, more preferably 2-30 amino acids in length, and most preferably 2-10 amino acids in length.

Exemplary linkers include linear peptides having at least two amino acid residues such as Gly-Gly, Gly-Ala-Gly, Gly-Pro-Ala, Gly-Gly-Gly-Gly-Ser (SEQ ID NO: 46). Exemplary linkers are presented herein as SEQ ID NOs: 12-13 (amino acid sequence) and SEQ ID NOs: 31-33, 40 and 41 (nucleotide sequence). Suitable linear peptides include polyglycine, polyserine, polyproline, polyalanine and oligopeptides consisting of alanyl and/or serinyl and/or prolinyl and/or glycyl amino acid residues.

Alternatively, the linker moiety may be a polypeptide multivalent linker that has branched “arms” that link multiple binding domain in a non-linear fashion. Examples include, but are not limited to, those disclosed in Tam (Journal of Immunological Methods 196:17, 1996). Preferably, a multivalent linker have between about three and about forty amino acid residues, all or some of which provide attachment sites for conjugation with binding domians. More preferably, the linker has between about two and about twenty attachment sites, which are often functional groups located in the amino acid residue side chains. However, alpha amino groups and alpha carboxylic acids can also serve as attachment sites. Exemplary multivalent linkers include, but are not limited to, polylysines, polyornithines, polycysteines, polyglutamic acid and polyaspartic acid. Optionally, amino acid residues with inert side chains, e.g., glycine, alanine and valine, can be included in the amino acid sequence. The linkers may also be a non peptide chemical entity such as a chemical linker is suitable for parenteral or oral administration once attached to the binding domains. The chemical linker may be a bifunctional linker, each of which reacts with a binding domain. Alternatively, the chemical linker may be a branched linker that has a multiplicity of appropriately spaced reactive groups, each of which can react with a functional group of a binding domain. The binding domains are attached by way of reactive functional groups and are spaces such that steric hindrance does not substantially interfere with formation of covalent bonds between some of the reactive functional groups (e.g., amines, carboxylic acids, alcohols, aldehydes and thiols) and the peptide. Not all attachment sites need be occupied. See e.g., Liu, et al., U.S. Application Serial No. 20030064053, expressly incorporated by refernce herein.

Ig-Like Domains of the Invention

The multivalent soluble receptor proteins of the invention are comprised of at least two Ig-like domains that bind at least two different angiogenic factor. The multivalent soluble receptor protein may also contain a multimerizing domain. The precise site at which the fusion is made is not critical; particular sites are well known and may be selected in order to optimize the biological activity, secretion, bioavailability or binding characteristics of the protein.

Examples of multivalent soluble receptor proteins that are provided by the present invention are described throughout and particularly in the examples and in FIGS. 1A-C and 2A-H. Ig-like domains are known and recognized by those skilled in the art. Briefly, they are generally characterized as containing about 110 amino acid residues and contain an intrachain disulfide bond that forms approximately 60 amino acid loop. (Immunology, Janis Kuby 1992, W.H Freeman & Company, New York) X-ray crystallography has revealed that Ig-like domains are usually folded into a compact structure, known as an immunoglobulin fold. This structure characteristically is comprised of two beta pleated sheets, each containing three or four antiparallel beta strands of amino acids (Kuby 1992).

Receptor Tyrosine Kinases (RTKs)

Receptor tyrosine kinases (RTKs) are transmembrane proteins that span the plasma membrane just once. Ligands that trigger RTKs include insulin, Vascular Endothelial Growth Factor (VEGF), Platelet-Derived Growth Factor (PDGF), Epidermal Growth Factor (EGF), Fibroblast Growth Factor (FGF) and Macrophage Colony-Stimulating Factor (MCSF).

Receptor tyrosine kinases (RTKs) are cell surface transmembrane proteins responsible for intracellular signal transduction which are activated by binding of a ligand to two adjacent receptors resulting in formation of an active dimer which catalyzes the phosphorylation of tyrosine residues. This activated dimer attaches phosphate groups to certain tyrosine residues converting them into an active state. The human genome encodes a large number of different tyrosine kinases, some of which act directly by transferring their phosphate to transcription factors thereby activating them. Receptor tyrosine kinases are involved in cellular signaling pathways and regulate key cell functions such as proliferation, differentiation, migration and invasion as well as angiogenesis. More than 70% of the known oncogenes and proto-oncogenes involved in cancer code for PTKs and over-expression and/or structural alteration of receptor tyrosine kinases has been associated with tumor growth, angiogenesis and metastasis.

VEGF (Vascular Endothelial Growth Factor)

A number of strategies aimed at blockage of the VEGF pathway are in clinical development. Blockage of the VEGF pathway has been achieved by a number of strategies such as blocking antibodies targeted against VEGF (Asano, M., et al. (1998) Hybridoma 17, 185-190) or its receptors (Prewett, M. et al. (1999) Cancer Res. 59, 5209-5218), soluble decoy receptors that prevent VEGF from binding to its normal receptors, as well as chemical inhibitors of the tyrosine kinase activity of the VEGFRs. Recently, a study that compared the efficacy of VEGF blockade to other “antiangiogenic” strategies established that this approach is superior to many others (Holash et al. PNAS, 99(17) 11393, 2002; WO 00/75319).

There are at least three recognized VEGF receptors: VEGFR1, VEGFR2 and VEGFR3. VEGFR1 is also called Flt-1, whose biological function is not well defined yet. Vascular Endothelial Growth Factor receptor 1 is also called_fms-related tyrosine kinase 1 (FLT1), and vascular endothelial growth factor/vascular permeability factor receptor. VEGFR2 is a transmembrane tyrosine kinase receptor, consisting of an Ig-like extracellular domain, a hydrophobic transmembrane domain, and an intracellular domain containing two tyrosine kinase motifs. VEGFR3 plays a key role in lymphatic angiogenesis. VEGFR3 binds VEGF-C and -D.

Vascular Endothelial Growth Factor (VEGF) mediates its actions through the VEGF receptor 1 (Flt-1) and VEGF receptor 2 (KDR or Flk-1) receptor tyrosine kinases. To localize the extracellular region of Flt-1 that is involved in ligand interactions, secreted Fc fusion proteins between the extracellular ligand biding domain of the receptor and IgG1 Fc have been generated and evaluated for VEGF-A and PlGF-1 affinity (Cunningham et al. 1997. Biochem Biophys Res Commun. 1997 Feb. 24; 231(3):596-9; Ma L et al. Biotechnol Appl Biochem. 34(Pt 3):199-204, 2001; Holash et al. Proc Natl Acad Sci USA. August 20; 99(17):11393-8 (2002)). Ligand binding studies show that amino acids 1-234 are sufficient to achieve minimal VEGF-A (VEGF 165 isoform) interactions. The extension of this region to 1-331 amino acids (SEQ ID NO:3) provides high affinity ligand binding comparable to the full receptor. This region is also sufficient to achieve interactions of Flt-1 with Placental Growth Factor (PIGF-1). VEGFR1 binds VEGF-A and -B.

VEGFR2 is also called KDR in human and Flk-1 for its mouse homologous. VEGFR2 (KDR/FLK-1) is a ˜210 kDa member of a receptor tyrosine kinase family whose activation plays a role in a large number of biological processes such as embryonic development, wound healing, cell proliferation, migration, and differentiation. VEGFR2 expression is mostly restricted to vascular endothelial cells. VEGFR2 binds VEGF-A and -B. The extracellular region of KDR consists of seven immunoglobulin-like domains, and deletion studies have shown that amino acids 1-327 (SEQ ID NO:6) are sufficient and necessary for high affinity binding to VEGF (Kaplan et al. 1997; Fu et al 1998). Deletion of amino acids 224-327 from this construct reduced the binding to VEGF by >1000-fold, indicating a critical functional role for this region in VEGF/KDR interaction. Results suggest that VEGFR-3 needs to be associated to VEGFR-2 to induce ligand-dependent cellular responses (Alam A. et al., Biochem Biophys Res Commun. 2004 Nov. 12; 324(2):909-15).

Vascular endothelial growth factor receptor-3 (VEGFR-3/Flt4) binds two known members of the VEGF ligand family, VEGF-C and VEGF-D, and has a critical function in the remodeling of the primary capillary vasculature of midgestation embryos. Later during development, VEGFR-3 regulates the growth and maintenance of the lymphatic vessels. VEGFR-3 is essential for vascular development and maintenance of lymphatic vessel's integrity (Alam A. et al., Biochem Biophys Res Commun. 2004 Nov. 12; 324(2):909-15). The VEGF-C binding region of the receptor has been determined by He et al. (2002) to be within amino acids 1-330 (amino acids 1-330 of SEQ ID NO:7).

One method for VEGF ligand blockade is the use of soluble VEGF receptors such as those derived from VEGFR-1 or VEGFR-2. One method for constructing these molecules involves fusing the extracellular IgG-like domains of the VEGF receptors that are responsible for binding the VEGF ligand, to the human IgG1 heavy chain fragment with a signal sequence at the N-terminus for secretion. Given the high degree of amino acid homology between Flt-1 and KDR, corresponding regions of amino acids between the 2 receptors can substitute when swapped between the molecules and in such a manner, create molecules with altered binding affinities. For example the KDR/Flt-1 hybrid VEGF-Trap. VEGF (Vascular Endothelial Growth Factor) Trap is a composite decoy receptor fusion protein that contains portions of the extracellular domains of two different VEGF receptors VEGFR-1 (flt-1) and VEGFR-2 (KDR). The VEGF Trap (R1R2) has a high affinity for VEGF (Holash et al. Proc Natl Acad Sci USA. August 20; 99(17):11393-8 (2002)).

Chimeric VEGF receptors which are chimeras of derived from VEGFR-2 and VEGFR-3 are described for example in WO02/060950.

Other Angiogenic Factors

Recent research has indicated that a number of growth factor pathways are involved in tumor progression (Rich and Bigner, Nat Rev Drug Discov. May; 3(5):430-46 (2004); Garcia-Echeverria and Fabbro. Mini Rev Med Chem. March; 4(3):273-83 (2004)). Of these factors the tyrosine kinase receptor family members fibroblast growth factor (FGF; Powers et al. Endocr Relat Cancer. 2000 September; 7(3):165-97), platelet derived growth factor (PDGF; Saharinen et al. J Clin Invest. 2003 May; 111 (9):1277-80; Ostman Cytokine & Growth Factor Reviews 15 (2004) 275-286), epidermal growth factor (EGF), hepatocyte growth factor (HGF) and Insulin-like growth factor (IGF) have been implicated.

Blocking ligands such as FGF, PDGF, EGF, angiopoietins (e.g. angiopoietin-1, angiopoietin-2), Ephrin ligands (e.g. Ephrin B2, A1, A2), IntegrinAV, Integrin B3, placental growth factor, tumor growth factor-alpha, tumor growth factor-beta, tumor necrosis factor-alpha and tumor necrosis factor-beta from binding to their receptors either alone or in addition to VEGF may lead to tumor stabilization or regression in cancer types that are unresponsive or not completely responsive to VEGF treatment alone.

Tyrosine kinase receptor/IgG fusions have been described for VEGF, PDGF, and FGF. Several groups have used these soluble receptors to block PDGF, FGF and VEGF binding to its respective ligand receptor to treat tumor growth in various animal models as a monotherapy (Strawn et al. 1994 J Biol Chem. August 19; 269(33):21215-22) and in combination (Ogawa et al. 2002 Cancer Gene Ther. August; 9(8):633-40). In all cases described the soluble receptors are delivered as either a monotherapy or in combination from separate viral constructs.

Platelet-Derived Growth Factor (PDGF)

Platelet-derived growth factor (PDGF), a factor released from platelets upon clotting, is responsible for stimulating the proliferation of fibroblasts in vitro. PDGF is also a mitogen for vascular smooth muscle cells, bone cells, cartilage cells, connective tissue cells and some blood cells (Hughes A, et al. Gen Pharmacol 27(7):1079-89, (1996)). PDGF is involved in many biological activities, including hyperplasia, chemotaxis, embryonic neuron fiber development, and respiratory tubule epithelial cells development.

The biological effects of platelet-derived growth factor (PDGF) are mediated by alpha- and beta-PDGF receptors (PDGFR alpha and β). The PDGFR alpha receptor binds PDGF-AA, AB, BB and CC ligands. Using deletion mutagenesis the PDGF-AA and -BB binding sites have been mapped to amino acids 1-314 of the PDGFR alpha receptor (SEQ ID NO:16; Lokker et al. J Biol Chem. 1997 Dec. 26; 272(52):33037-44, 1997; Miyazawa et al. J Biol Chem. 1998 Sep. 25; 273(39):25495-502, 1998; Mahadevan et al. J Biol Chem. 1995 Nov. 17; 270(46):27595-600, 1995).

The biological effects of platelet-derived growth factor (PDGF) are mediated by alpha- and beta-PDGF receptors (PDGFR alpha and β). The PDGFRβ receptor binds PDGF-BB and DD ligands. Using deletion mutagenesis the PDGF-BB binding sites have been mapped to amino acids 1-315 of the PDGFRβ receptor (SEQ ID NO: 19; Lokker et al. J Biol Chem. 1997 Dec. 26; 272(52):33037-44, 1997).

Fibroblast Growth Factor Receptors (FGFRs)

Most FGFs initiate fibroblast proliferation, however, they also induce proliferation of endothelial cells, chondrocytes, smooth muscle cells, and melanocytes, etc. Furthermore, FGF-2 molecule has been shown to induce adipocyte differentiation, stimulates astrocyte migration and prolongs neuron survival (Burgess, W. H. and T. Maciag Annu. Rev. Biochem. 58:575, 1989). Four fibroblast growth factor receptors (FGFR1-4) constitute a family of transmembrane tyrosine kinases that serve as high affinity receptors for at least 22 FGF ligands. Gene targeting in mice has yielded valuable insights into the functions of this important gene family in multiple biological processes. These include mesoderm induction and patterning; cell growth, migration, and differentiation; organ formation and maintenance; neuronal differentiation and survival; wound healing; and malignant transformation. In relation to FGFR1, structure binding studies have revealed that amino acids 119-372 of the receptor are required for acidic and basic FGF binding (SEQ ID NO:22; Challaiah et al., 1999; Olsen et al., 2004).

For FGFR2, structure binding studies have revealed that amino acids 126-373 of the receptor (SEQ ID NO:25) are required for FGF binding (Miki et al., Science. 1991 Jan. 4; 251(4989):72-5, 1991; 1992; Celli et al., EMBO J. 1998 Mar. 16; 17(6):1642-55, 1998).

In addition, amino acid substitutions based upon naturally occurring human mutations can be introduced into the FGFR2 binding region to improve ligand affinity or specificity. For example, Apert syndrome (AS) is characterized by craniosynostosis (premature fusion of cranial sutures) and severe syndactyly of the hands and feet. Two activating mutations, Ser-252-->Trp and Pro-253-->Arg, in FGFR2 account for nearly all known cases of AS. These mutations introduce additional interactions between FGFR2 and FGF2, thereby augmenting FGFR2-FGF2 affinity. The Pro-253-->Arg mutation will indiscriminately increase the affinity of FGFR2 toward any FGF. In contrast, the Ser-252-->Trp mutation will selectively enhance the affinity of FGFR2 toward a limited subset of FGFs (Ibrahimi et al., Proc Natl Acad Sci USA. 2001 Jun. 19; 98(13):7182-7, 2001).

HGF Ligand/Receptor Family

Hepatocyte growth factor (HGF) was originally described as a mitogenic factor of hepatocytes during liver regeneration, but HGF has a variety of biological activities including mitogenesis and morphogenesis in epithelial cells. HGF is essential for normal embryological development and liver regeneration. The receptor of HGF, c-Met, is also a tyrosine kinase receptor. Also, over expression of c-Met and its activation by autocrine HGF expression is found in a variety of human tumors indicating co-expression of HGF and c-Met may be involved in tumor metastasis. (Sakkab D. et al., J Biol Chem, Vol. 275(12) 8806-8811, 2000). Met, the receptor for hepatocyte growth factor (HGF), is activated in human cancer by both ligand-dependent and -independent mechanisms. Hepatocyte growth factor (HGF) binds the extracellular domain of C-Met and activates the Met receptor to induce mitogenesis, morphogenesis, and motility. The extracellular domain of Met is comprised of Sema, PSI, and four IPT subdomains. Observations indicate that only the Sema domain and following PSI domain of the extracellular region of the receptor (SEQ ID NO:28; amino acids 1-562) is necessary for dimerization in addition to HGF binding (Kong-Beltran et al., Cancer Cell. 2004 July; 6(1):75-84, 2004; Trusolino L, Comoglio P M., Nat Rev Cancer. 2002 April; 2(4):289-300).

Angiopoietins (e.g. Angiopoietin-1, Angiopoietin-2)

Tie2 (Tek) is the receptor for Angiopoietins 1 & 2 (Ang1 and Ang2) Angiopoietins act as endothelial growth factors. Ang1 promotes angiogenesis by activating Tie2. Ang2 may also activate Tie2 depending on local conditions (I've added Tie2 to the sequence listing file).

Angiopoietin (Ang) 1, a ligand for the receptor tyrosine kinase Tie2, regulates the formation and stabilization of the blood vessel network during embryogenesis. In adults, Ang1 is associated with blood vessel stabilization and recruitment of perivascular cells, whereas Ang2 acts to counter these actions. Recent results from gene-targeted mice have shown that Ang2 is also essential for the proper patterning of lymphatic vessels and that Ang1 can be substituted for this function. This receptor possesses a unique extracellular domain containing 2 immunoglobulin-like loops separated by 3 epidermal growth factor-like repeats that are connected to 3 fibronectin type III-like repeats. Studies have indicated that the extracellular region of the Tie2 receptor (amino acids 1-733) is capable of ligand binding (Lin P et al., Proc Natl Acad Sci USA. 1998 Jul. 21; 95(15):8829-34; Lin P, et al.” J Clin Invest. 1997 Oct. 15; 100(8):2072-8.

Exemplary binding domains for use in construction of the multivalent soluble receptor proteins of the invention are described in Table 1, below. Binding of the ligand may not be the only variable that would be important, since other factors such as the secretion of the receptor from the cell, dimerization and bioavailability become important.

It is understood that variants or mutants of the Ig-like domains that bind to an angiogenic factor(s) find use in the present invention. For in vivo an even in vitro applications in order to inhibit angiogenesis the multivalent soluble receptor proteins of the invention need to be available for binding to the angiogenic factors. It is believed that positive charges on proteins allow proteins to bind to extracellular matrix components and the like, possibly reducing their availability to bind their ligand (e.g. angiogenic factor). Therefore, the invention also provides modified multivalent soluble receptor proteins that are modified to reduce the positive charges (e.g. lower the pI). There are methods known to those skilled in the art for modifying the charge of a protein including acetylation and/or by replacing codons of the coding region that code for positive charged amino acids with codons for neutral or negatively charged amino acids. Examples of these types of modifications are described in WO200075319. Various amino acid substitutions can be made in the Ig-like domain or domains without departing from the spirit of the present invention with respect to the proteins' ability to bind to angiogenic factors and inhibit angiogenesis. Thus point mutations and broader variations may be made in the Ig-like domain(s) so as to impart interesting properties that do not substantially affect the chimeric protein's ability to bind angiogenic factors and inhibit angiogenesis. Sequence variants encoding the Ig-like domains of the multivalent soluble receptor proteins of the invention are included within the scope of the invention.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J. Mol. Biol. 215: 403-410 (1990), with software that is publicly available through the National Center for Biotechnology Information (http://www.ncbi.nim.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981).

In accordance with the present invention, also encompassed are sequence variants of genes encoding an Ig-like domain of a multivalent soluble receptor protein of the invention that have 80, 85, 88, 89, 90, 91, 92, 93, 94, 95, 96, 97, 98 or 99% or more sequence identity to the native nucleotide or amino acid sequence of an anti-cancer compound described herein. Sequence variants include nucleotide sequences that encode the same polypeptide as is encoded by the therapeutic compounds or factors described herein. Thus, where the coding frame of the Ig-like domain is known, it will be appreciated that as a result of the degeneracy of the genetic code, a number of coding sequences can be produced. For example, the triplet CGT encodes the amino acid arginine. Arginine is alternatively encoded by CGA, CGC, CGG, AGA, and AGG. Therefore it is appreciated that such substitutions in the coding region fall within the sequence variants that are covered by the present invention.

A nucleic acid sequence is considered to be “selectively hybridizable” to a reference nucleic acid sequence if the two sequences specifically hybridize to one another under moderate to high stringency hybridization and wash conditions (i.e. “stringent hybridization conditions” and “stringent wash conditions). Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

“Stringent hybridization conditions” and “stringent wash conditions” in the context of nucleic acid hybridization experiments such as Southern and Northern hybridizations are sequence dependent, and are different under different environmental parameters. Longer sequences hybridize at higher temperatures. An extensive guide to the hybridization of nucleic acids is found in Tijssen (1993) Laboratory Techniques in Biochemistry and Molecular Biology-Hybridization with Nucleic Acid Probes part 1 chapter 2 “Overview of principles of hybridization and the strategy of nucleic acid probe assays”, Elsevier, N.Y. Generally, highly stringent hybridization and wash conditions are selected to be about 5° C. to 10° C. (preferably 5° C.) lower than the thermal melting point (Tₘ) for the specific sequence at a defined ionic strength and pH. Typically, under highly stringent conditions a probe will hybridize to its target subsequence, but to no other unrelated sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids that have more than 100 complementary residues on a filter in a Southern or Northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash conditions for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization.

Sequence variants that encode a polypeptide with the same biological activity as an Ig-like domain of a multivalent soluble receptor protein of the invention, as described herein, and hybridize under moderate to high stringency hybridization conditions are considered to be within the scope of the present invention. It is further appreciated that such sequence variants may or may not hybridize to the parent sequence under conditions of high stringency. This would be possible, for example, when the sequence variant includes a different codon for each of the amino acids encoded by the parent nucleotide. Such variants are, nonetheless, specifically contemplated and encompassed by the present invention.

It will be appreciated that various amino acid substitutions can be made in the Ig-like domain or domains of the chimeric VEGF receptor proteins of the present invention without departing from the spirit of the present invention with respect to the chimeric proteins' ability to bind to and inhibit angiogenesis or lymphangiogenesis. Thus, point mutational and other broader variations may be made in a multivalent soluble receptor protein of the invention so as to impart interesting properties that do not substantially affect the protein's ability to bind to and inhibit angiogenesis or lymphangiogenesis. These variants may be made by means generally known well in the art.

Amino acid sequence variants of the Ig-like domain or domains present in the multivalent soluble receptor proteins of the present invention can also be prepared by creating mutations in the DNA encoding the protein. Such variants include, for example, deletions from, or insertions or substitutions of, amino acid residues within the amino acid sequence of the Ig-like domain or domains. Any combination of deletion, insertion, and substitution may also be made to arrive at the final construct, provided that the final construct possesses the desired activity. Obviously, the mutations that will be made in the DNA encoding the variant must not place the sequence out of reading frame and preferably will not create complementary regions that could produce secondary mRNA structure (see, e.g., EP 75,444A).

At the genetic level, variants of the Ig-like domain or domains present in the multivalent soluble receptor proteins of the present invention ordinarily are prepared by site-directed mutagenesis of nucleotides in the DNA encoding an IgG-like domain or domains, thereby producing DNA encoding the variant, and thereafter expressing the DNA in recombinant cell culture or in vivo. The variants typically exhibit the same qualitative ability to bind to the ligand as does the unaltered soluble receptor protein.

Gene Delivery Vectors

The present invention contemplates the use of any vector for introduction of one or more coding sequences for a multivalent soluble receptor protein into mammalian cells. Exemplary vectors include but are not limited to, viral and non-viral vectors, such as retroviruses (e.g. derived from MoMLV, MSCV, SFFV, MPSV, SNV etc), including lentiviruses (e.g. derived from HIV-1, HIV-2, SIV, BIV, FIV etc.), adenovirus (Ad) vectors including replication competent, replication deficient and gutless forms thereof, adeno-associated virus (AAV) vectors, simian virus 40 (SV-40) vectors, bovine papilloma virus vectors, Epstein-Barr virus vectors, herpes virus vectors, vaccinia virus vectors, Moloney murine leukemia virus vectors, Harvey murine sarcoma virus vectors, murine mammary tumor virus vectors, Rous sarcoma virus vectors, baculovirus vectors and nonviral plasmid vectors. In one approach, the vector is a viral vector. Viruses can efficiently transduce cells and introduce their own DNA into a host cell. In generating recombinant viral vectors, a gene or coding sequence for a heterologous (or non-native) protein may be incorporated into the viral vector.

In one case, viral vectors are constructed by replacing non-essential genes with one or more genes encoding one or more heterologous gene products (e.g. RNA, protein). The vector may or may not also comprise a “marker” or “selectable marker” function by which the vector can be identified and selected. While any selectable marker can be used, selectable markers for use in such expression vectors are generally known in the art and the choice of the proper selectable marker will depend on the host cell and application. Examples of selectable marker genes which encode proteins that confer resistance to antibiotics or other toxins include ampicillin, methotrexate, tetracycline, neomycin (Southern et al., J., J Mol Appl Genet. 1982; 1(4):327-41 (1982)), mycophenolic acid (Mulligan et al., Science 209:1422-7 (1980)), puromycin, zeomycin, hygromycin (Sugden et al., Mol Cell Biol. 5(2):410-3 (1985)) or G418.

As will be understood by those of skill in the art, expression vectors typically include an origin of replication, a promoter operably linked to the coding sequence or sequences to be expressed, as well as ribosome binding sites, RNA splice sites, a polyadenylation site, and transcriptional terminator sequences, as appropriate to the coding sequence(s) being expressed. Control sequences are nucleotide sequences necessary for the expression of an operably linked coding sequence in a particular host organism. The control sequences that are suitable for prokaryotes, for example, include a promoter, optionally an operator sequence, a ribosome binding site, etc. Eukaryotic cells are known to utilize promoters, polyadenylation signals, and enhancers.

Reference to a vector or other DNA sequences as “recombinant” merely acknowledges the operable linkage of DNA sequences which are not typically operatively linked as isolated from or found in nature. Regulatory (expression/control) sequences are operatively linked to a nucleotide sequence when the expression/control sequences regulate the transcription and, as appropriate, translation of the nucleotide sequence. Thus expression/control sequences can include promoters, enhancers, transcription terminators, a start codon (i.e., ATG) in front of a coding sequence, splicing signal for introns and stop codons.

The vectors of the invention typically include heterologous control sequences, including, but not limited to, constitutive promoters, tissue or cell type specific promoters, tumor selective promoters and enhancers, regulatable or inducible promoters, enhancers, and the like.

Exemplary promoters include, but are not limited to: the cytomegalovirus (CMV) immediate early promoter, the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter (Ill et al., Blood Coagul. Fibrinolysis 8S2:23-30 (1997), chimeric liver-specific promoters (LSPs), the E2F promoter, the telomerase (hTERT) promoter; the cytomegalovirus enhancer/chicken beta-actin/Rabbit β-globin promoter (CAG promoter; Niwa H. et al. 1991. Gene 108(2):193-9) and the elongation factor 1-alpha promoter (EF1-alpha) promoter (Kim D W et al. 1990. Gene. 91(2):217-23 and Guo Z S et al. 1996. Gene Ther. 3(9):802-10. Preferred promoters include the EF1-alpha promoter, the PGK promoter, a cytomegalovirus immediate early gene (CMV) promoter and a cytomegalovirus enhancer/chicken beta-actin (CAG) promoter. The nucleotide sequence of these and numerous additional promoters are known in the art. The relevant sequences may be readily obtained from public databases and incorporated into vectors for use in practicing the present invention.

Secondary coding sequences may be used to enhance expression. For example, dihydrofolate reductase (DHFR) may be used to amplify expression in cell culture whereby expression is controlled by altering the methotrexate (MTX), concentration.

The present invention also contemplates the inclusion of a gene regulation system for the controlled expression of immunoglobulin coding sequences. Gene regulation systems are useful in the modulated expression of a particular gene or genes. In one exemplary approach, a gene regulation system or switch includes a chimeric transcription factor that has a ligand binding domain, a transcriptional activation domain and a DNA binding domain. The domains may be obtained from virtually any source and may be combined in any of a number of ways to obtain a novel protein. A regulatable gene system also includes a DNA response element which interacts with the chimeric transcription factor. This element is located adjacent to the gene to be regulated.

Exemplary gene regulation systems that may be employed in practicing the present invention include, the Drosophila ecdysone system (Yao et al., Proc. Nat. Acad. Sci., 93:3346 (1996)), the Bombyx ecdysone system (Suhr et al., Proc. Nat. Acad. Sci., 95:7999 (1998)), the Valentis GeneSwitch® synthetic progesterone receptor system which employs RU-486 as the inducer (Osterwalder et al., Proc Natl Acad Sci 98(22):12596-601 (2001)); the TetÔ & RevTetÔ Systems (BD Biosciences Clontech), which employs small molecules, such as tetracycline (Tc) or analogues, e.g. doxycycline or anhydrotetracycline, to regulate (turn on or off) transcription of the target (Knott et al., Biotechniques 32(4):796, 798, 800 (2002)); ARIAD Regulation Technology which is based on the use of a small molecule to bring together two intracellular molecules, each of which is linked to either a transcriptional activator or a DNA binding protein. When these components come together, transcription of the gene of interest is activated. Ariad has two major systems: a system based on homodimerization and a system based on heterodimerization (Rivera et al., Nature Med, 2(9):1028-1032 (1996); Ye et al., Science 283: 88-91 (2000)), both of which may be employed in practicing the present invention.

Preferred gene regulation systems for use in practicing the present invention are the ARIAD Regulation Technology and the TetÔ & RevTetÔ Systems.

AAV Vectors

Adeno-associated virus (AAV) is a helper-dependent human parvovirus which is able to infect cells latently by chromosomal integration. AAV vectors have significant potential as gene transfer vectors because of their non-pathogenic nature, excellent clinical safety profile and ability to direct significant amounts of transgene expression in vivo. Recombinant AAV vectors are characterized in that they are capable of directing the expression and the production of the selected transgenic products in targeted cells. Thus, the recombinant vectors comprise at least all of the sequences of AAV essential for encapsidation and the physical structures for infection of target cells. Infection of a cell with an AAV viral vector incorporated into a viral particle, typically leads to integration of the viral vector into the host cell genome. Therefore, AAV vectors provide the potential for long term expression from the cell, and “daughter cells” that are a result of cell division.

The present invention contemplates the use of any AAV viral vector serotype for introduction of constructs comprising the coding sequence for immunoglobulin heavy and light chains and a self processing cleavage sequence into cells so long as expression of immunoglobulin results. A large number of AAV vectors are known in the art. In generating recombinant AAV viral vectors, non-essential genes are replaced with a gene encoding a protein or polypeptide of interest. Early work was carried out using the AAV2 serotype. However, the use of alternative AAV serotypes other than AAV2 (Davidson et al (2000), PNAS 97(7)3428-32; Passini et al (2003), J. Virol 77(12):7034-40) has demonstrated different cell tropisms and increased transduction capabilities. In one aspect, the present invention is directed to AAV vectors and methods that allow optimal AAV vector-mediated delivery and expression of an immunoglobulin or other therapeutic compound in vitro or in vivo.

For use in practicing the present invention rAAV virions may be produced using standard methodology, known to those of skill in the art and are constructed such that they include, as operatively linked components in the direction of transcription, control sequences including transcription initiation and termination sequences, the immunoglobulin coding sequence(s) of interest and a self processing cleavage sequence. More specifically, the recombinant AAV vectors of the instant invention comprise: (1) a packaging site enabling the vector to be incorporated into replication-defective AAV virions; (2) the coding sequence for two or more polypeptides or proteins of interest, e.g., heavy and light chains of an immunoglobulin of interest; and (3) a sequence encoding a self-processing cleavage site alone or in combination with an additional proteolytic cleavage site. AAV vectors for use in practicing the invention are constructed such that they also include, as operatively linked components in the direction of transcription, control sequences including transcription initiation and termination sequences. These components are flanked on the 5′ and 3′ end by functional AAV ITR sequences. By “functional AAV ITR sequences” is meant that the ITR sequences function as intended for the rescue, replication and packaging of the AAV virion.

Recombinant AAV vectors are also characterized in that they are capable of directing the expression and production of recombinant immunoglobulins in target cells. Thus, the recombinant vectors comprise at least all of the sequences of AAV essential for encapsidation and the physical structures for infection of the recombinant AAV (rAAV) virions. Hence, AAV ITRs for use in the vectors of the invention need not have a wild-type nucleotide sequence (e.g., as described in Kotin, Hum. Gene Ther., 5:793-801, 1994), and may be altered by the insertion, deletion or substitution of nucleotides or the AAV ITRs may be derived from any of several AAV serotypes. Generally, an AAV vector is a vector derived from an adeno-associated virus serotype, including without limitation, AAV-1, AAV-2, AAV-3, AAV-4, AAV-5, AAV-6, AAV-7, AAV-8, etc. Preferred rAAV vectors have the wild type REP and CAP genes deleted in whole or part, but retain functional flanking ITR sequences. Table 2 illustrates exemplary AAV serotypes for use in practicing the present invention.

TABLE 2
Exemplary AAV Serotypes For Use In Gene Transfer.

| | | | | Immunity |
|---|---|---|---|---|
| | | Genome | Homology to | in Human |
| Serotype | Origin | Size (bp) | AAV2 | Population |
| AAV-1 | Human specimen | 4718 | NT: 80% | NAB: 20% |
| | | | AA: 83% | |
| AAV-2 | Human Genital | 4681 | NT: 100% | NAB: 27-53% |
| | Abortion Tissue | | AA: 100% | |
| | Amnion Fluid | | | |
| AAV-3 | Human | 4726 | NT: 82% | cross reactivity |
| | Adenovirus | | AA: 88% | with AAV2 |
| | Specimen | | | NAB |
| AAV-4 | African Green | 4774 | NT: 66% | Unknown |
| | Monkey | | AA: 60% | |
| AAV-5 | Human Genital | 4625 | NT: 65% | ELISA: 45% |
| | Lesion | | AA: 56% | NAB: 0% |
| AAV-6 | Laboratory Isolate | 4683 | NT: 80% | 20% |
| | | | AA: 83% | |
| AAV-7 | Isolated From | 4721 | NT: 78% | NAB: <1:20 |
| | Heart DNA of | | AA: 82% | (˜5%) |
| | Rhesus Monkey | | | |
| AAV-8 | Isolated From | 4393 | NT: 79% | NAB: <1:20 |
| | Heart DNA of | | AA: 83% | (˜5%) |
| | Rhesus Monkey | | | |

Typically, an AAV expression vector is introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large Rep proteins (Rep78 and Rep68), typically by mutating the start codon following p5 from ATG to ACG, as described in U.S. Pat. No. 6,548,286, expressly incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety.

More than 40 serotypes of AAV are currently known, however, new serotypes and variants of existing serotypes are still being identified today and are considered within the scope of the present invention. See Gao et al (2002), PNAS 99(18):11854-6; Gao et al (2003), PNAS 100(10):6081-6; Bossis and Chiorini (2003), J. Virol. 77(12):6799-810). Different AAV serotypes are used to optimize transduction of particular target cells or to target specific cell types within a particular target tissue. The use of different AAV serotypes may facilitate targeting of diseased tissue. Particular AAV serotypes may more efficiently target and/or replicate in specific target tissue types or cells. A single self-complementary AAV vector can be used in practicing the invention in order to increase transduction efficiency and result in faster onset of transgene expression (McCarty et al., Gene Ther. 2001 August; 8(16): 1248-54).

In practicing the invention, host cells for producing rAAV virions include mammalian cells, insect cells, microorganisms and yeast. Host cells can also be packaging cells in which the AAV rep and cap genes are stably maintained in the host cell or producer cells in which the AAV vector genome is stably maintained and packaged. Exemplary packaging and producer cells are derived from 293, A549 or HeLa cells. AAV vectors are purified and formulated using standard techniques known in the art.

Retroviral and Lentiviral Vectors

Retroviral vectors are a common tool for gene delivery (Miller, 1992, Nature 357: 455-460). Retroviral vectors including lentiviral vectors may be used in practicing the present invention. Retroviral vectors have been tested and found to be suitable delivery vehicles for the stable introduction of a variety of genes of interest into the genomic DNA of a broad range of target cells. The ability of retroviral vectors to deliver unrearranged, a transgene(s) into cells makes retroviral vectors well suited for transferring genes into cells. Further, retroviruses enter host cells by the binding of retroviral envelope glycoproteins to specific cell surface receptors on the host cells. Consequently, pseudotyped retroviral vectors in which the encoded native envelope protein is replaced by a heterologous envelope protein that has a different cellular specificity than the native envelope protein (e.g., binds to a different cell-surface receptor as compared to the native envelope protein) may also find utility in practicing the present invention.

The present invention provides retroviral vectors which include e.g., retroviral transfer vectors comprising one or more sequences which encode a multivalent soluble receptor protein of the invention and retroviral packaging vectors comprising one or more packaging elements. In particular, the present invention provides pseudotyped retroviral vectors encoding a heterologous or functionally modified envelope protein for producing pseudotyped retrovirus.

The core sequence of the retroviral vectors of the present invention may be readily derived from a wide variety of retroviruses, including for example, B, C, and D type retroviruses as well as spumaviruses and lentiviruses (see RNA Tumor Viruses, Second Edition, Cold Spring Harbor Laboratory, 1985). An example of a retrovirus suitable for use in the compositions and methods of the present invention includes, but is not limited to, lentivirus. Other retroviruses suitable for use in the compositions and methods of the present invention include, but are not limited to, Avian Leukosis Virus, Bovine Leukemia Virus, Murine Leukemia Virus, Mink-Cell Focus-Inducing Virus, Murine Sarcoma Virus, Reticuloendotheliosis virus and Rous Sarcoma Virus. Particularly preferred Murine Leukemia Viruses include 4070A and 1504A (Hartley and Rowe, J. Virol. 19:19-25, 1976), Abelson (ATCC No. VR-999), Friend (ATCC No. VR-245), Graffi, Gross (ATCC No. VR-590), Kirsten, Harvey Sarcoma Virus and Rauscher (ATCC No. VR-998), and Moloney Murine Leukemia Virus (ATCC No. VR-190). Such retroviruses may be readily obtained from depositories or collections such as the American Type Culture Collection (“ATCC”; Rockville, Md.), or isolated from known sources using commonly available techniques.

Preferably, a retroviral vector sequence of the present invention is derived from a lentivirus. A preferred lentivirus is a human immunodeficiency virus, e.g., type 1 or 2 (i.e., HIV-1 or HIV-2, wherein HIV-1 was formerly called lymphadenopathy associated virus 3 (HTLV-III) and acquired immune deficiency syndrome (AIDS)-related virus (ARV)), or another virus related to HIV-1 or HIV-2 that has been identified and associated with AIDS or AIDS-like disease. Other lentivirus vectors that ,ay be used in practicing the invention include, a sheep Visna/maedi virus, a feline immunodeficiency virus (FIV), a bovine lentivirus (e.g. BIV; WO200366810), simian immunodeficiency virus (SIV), an equine infectious anemia virus (EIAV), and a caprine arthritis-encephalitis virus (CAEV).

The various genera and strains of retroviruses suitable for use in the compositions and methods are well known in the art (see, e.g., Fields Virology, Third Edition, edited by B. N. Fields et al., Lippincott-Raven Publishers (1996), see e.g., Chapter 58, Retroviridae: The Viruses and Their Replication, Classification, pages 1768-1771).

The present invention provides retroviral packaging systems for generating producer cells and producer cell lines that produce retroviruses, and methods of making such packaging systems. Accordingly, the present invention also provides producer cells and cell lines generated by introducing a retroviral transfer vector into such packaging systems (e.g., by transfection or infection), and methods of making such packaging cells and cell lines.

The retroviral packaging systems for use in practicing the present invention comprise at least two packaging vectors: a first packaging vector which comprises a first nucleotide sequence comprising a gag, a pol, or gag and pol genes; and a second packaging vector which comprises a second nucleotide sequence comprising a heterologous or functionally modified envelope gene. In one embodiment, the retroviral elements are derived from a lentivirus, such as HIV. Preferably, the vectors lack a functional tat gene and/or functional accessory genes (vif, vpr, vpu, vpx, nef). In another embodiment, the system further comprises a third packaging vector that comprises a nucleotide sequence comprising a rev gene. The packaging system can be provided in the form of a packaging cell that contains the first, second, and, optionally, third nucleotide sequences.

The invention is applicable to a variety of retroviral systems, and those skilled in the art will appreciate the common elements shared across differing groups of retroviruses. The description herein uses lentiviral systems as a representative example. However, all retroviruses share the features of enveloped virions with surface projections and containing one molecule of linear, positive-sense single stranded RNA, a genome consisting of a dimer, and the common proteins gag, pol and env.

Lentiviruses share several structural virion proteins in common, including the envelope glycoproteins SU (gp120) and TM (gp41), which are encoded by the env gene; CA (p24), MA (p17) and NC (p7-11), which are encoded by the gag gene; and RT, PR and IN encoded by the pol gene. HIV-1 and HIV-2 contain accessory and other proteins involved in regulation of synthesis and processing virus RNA and other replicative functions. The accessory proteins, encoded by the vif, vpr, vpu/vpx, and nef genes, can be omitted (or inactivated) from the recombinant system. In addition, tat and rev can be omitted or inactivated, e.g., by mutation or deletion.

In one embodiment, the lentiviral vector packaging systems provide separate packaging constructs for gag/pol and env, and typically employ a heterologous or functionally modified envelope protein (e.g. VSVG envelope). In a further embodiment, lentiviral vector systems have the accessory genes, vif, vpr, vpu and nef, deleted or inactivated. In a further embodiment, the lentiviral vector systems have the tat gene deleted or otherwise inactivated (e.g., via mutation). In another embodiment, the gag and pol coding sequence are “split” in to two separate coding sequences or open reading frames as known in the art. Typically the split gag and pol coding sequences are operatively linked to separate promoters and may be located on different nucleotide sequences.

Compensation for the regulation of transcription normally provided by tat can be provided by the use of a strong constitutive promoter, such as the human cytomegalovirus immediate early (HCMV-IE) enhancer/promoter. Other promoters/enhancers can be selected based on strength of constitutive promoter activity, specificity for target tissue (e.g., liver-specific promoter), or other factors relating to desired control over expression, as is understood in the art. For example, in some embodiments, it is desirable to employ an inducible promoter such as tet to achieve controlled expression. The gene encoding rev is preferably provided on a separate expression construct, such that the lentiviral vector system will involve four constructs (e.g. plasmids): one each for gag/pol, rev, envelope and the transfer vector. Regardless of the generation of the packaging system employed, gag and pol can be provided on a single construct or on separate constructs.

Typically, the packaging vectors are included in a packaging cell, and are introduced into the cell via transfection, transduction or infection. Methods for transfection, transduction or infection are well known by those of skill in the art. A retroviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some embodiments, the packaging vectors are introduced into the cells together with a dominant selectable marker, such as neo, DHFR, Gln synthetase or ADA, followed by selection in the presence of the appropriate drug and isolation of clones. A selectable marker gene can be linked physically to genes encoding by the packaging vector or may co-introduced (e.g. cotransfected) with the packaging vector.

Typically, the packaging vectors are included in a packaging cell, and are introduced into the cell via transfection, transduction or infection. Methods for transfection, transduction or infection are well known by those of skill in the art. A retroviral transfer vector of the present invention can be introduced into a packaging cell line, via transfection, transduction or infection, to generate a producer cell or cell line. The packaging vectors of the present invention can be introduced into human cells or cell lines by standard methods including, e.g., calcium phosphate transfection, lipofection or electroporation. In some embodiments, the packaging vectors are introduced into the cells together with a dominant selectable marker, such as neo, DHFR, Gln synthetase or ADA, followed by selection in the presence of the appropriate drug and isolation of clones. A selectable marker gene can be linked physically to genes encoding by the packaging vector or may co-introduced (e.g. cotransfected) with the packaging vector.

Stable cell lines, wherein the packaging functions are configured to be expressed by a suitable packaging cell, are known. For example, see U.S. Pat. No. 5,686,279; and Ory et al., Proc. Natl. Acad. Sci. (1996) 93:11400-11406, which describe packaging cells. Further description of stable cell line production can be found in Dull et al., 1998, J. Virology 72(11):8463-8471; and in Zufferey et al., 1998, J. Virology 72(12):9873-9880.

Zufferey et al., 1997, Nature Biotechnology 15:871-875, teach a lentiviral packaging plasmid wherein sequences 3′ of pol including the HIV-1 envelope gene are deleted. The construct contains tat and rev sequences and the 3′ LTR is replaced with poly A sequences. The 5′ LTR and psi sequences are replaced by another promoter, such as one which is inducible. For example, a CMV promoter or derivative thereof can be used.

The packaging vectors of interest may contain additional changes to the packaging functions to enhance lentiviral protein expression and to enhance safety. For example, all of the HIV sequences upstream of gag can be removed. Also, sequences downstream of envelope can be removed. Moreover, steps can be taken to modify the vector to enhance the splicing and translation of the RNA.

Optionally, a conditional packaging system is used, such as that described by Dull et al., 1998, J. Virology 72(11):8463-8471. Also preferred is the use of a self-inactivating vector (SIN), which improves the biosafety of the vector by deletion of the HIV-1 long terminal repeat (LTR) as described, for example, by Zufferey et al., 1998, J. Virology 72(12):9873-9880. Inducible vectors can also be used, such as through a tet-inducible LTR.

Adenoviral Vectors

Adenovirus gene therapy vectors are known to exhibit strong expression in vitro and in vivo, excellent titer, and the ability to transduce dividing and non-dividing cells in vivo (Hitt et al., Adv in Virus Res 55:479-505 (2000)).

As used herein, the terms “adenovirus” and “adenoviral particle” are used to include any and all viruses that may be categorized as an adenovirus, including any adenovirus that infects a human or an animal, including all known and later discovered groups, subgroups, and serotypes. Thus, as used herein, “adenovirus” and “adenovirus particle” refer to the virus itself or derivatives thereof and cover all serotypes and subtypes and both naturally occurring and recombinant forms, except where indicated otherwise. Such adenoviruses may be wildtype or may be modified in various ways known in the art or as disclosed herein. Such modifications include modifications to the adenovirus genome that are packaged in the particle in order to make an infectious virus. Such modifications include deletions known in the art, such as deletions in one or more of the adenoviral genes that are essential for replication, e.g., the E1a, E1b, E2a, E2b, E3, or E4 coding regions. The term “gene essential for replication” refers to a nucleotide sequence whose transcription is required for a viral vector to replicate in a target cell. For example, in an adenoviral vector of the invention, a gene essential for replication may be selected from the group consisting of the E1a, E1 b, E2a, E2b, and E4 genes. The terms also include replication-specific adenoviruses; that is, viruses that preferentially replicate in certain types of cells or tissues but to a lesser degree or not at all in other types. Such viruses are sometimes referred to as “cytolytic” or “cytopathic” viruses (or vectors), and, if they have such an effect on neoplastic cells, are referred to as “oncolytic” viruses (or vectors).

The adenoviral vectors of the invention include replication incompetent (defective) and replication competent vectors. Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein.

In the context of adenoviral vectors, the term “5′” is used interchangeably with “upstream” and means in the direction of the left inverted terminal repeat (ITR). In the context of adenoviral vectors, the term “3′” is used interchangeably with “downstream” and means in the direction of the right ITR.

Standard systems for generating adenoviral vectors for expression of inserted sequences are known in the art and are available from commercial sources, for example the Adeno-X™ expression system from Clontech (Clontechniques (January 2000) p. 10-12).

The present invention contemplates the use of any and all adenoviral serotypes to construct adenoviral vectors and virus particles according to the present invention. Adenoviral stocks that can be employed according to the invention include any adenovirus serotype. Adenovirus serotypes 1 through 47 are currently available from American Type Culture Collection (ATCC, Manassas, Va.), and the invention includes any other serotype of adenovirus available from any source. The adenoviruses that can be employed according to the invention may be of human or non-human origin. For instance, an adenovirus can be of subgroup A (e.g., serotypes 12, 18, 31), subgroup B (e.g., serotypes 3, 7, 11, 14, 16, 21, 34, 35), subgroup C (e.g., serotypes 1, 2, 5, 6), subgroup D (e.g., serotypes 8, 9, 10, 13, 15, 17, 19, 20, 22-30, 32, 33, 36-39, 42-47), subgroup E (serotype 4), subgroup F (serotype 40,41), or any other adenoviral serotype. Throughout the specification reference is made to specific nucleotides in adenovirus type 5. One skilled in the art can determine the corresponding nucleotides in other serotypes and therefore construct similar adenoviral vectors in other adenovirus serotypes. In one preferred embodiment, the adenoviral nucleotide sequence backbone is derived from adenovirus serotype 2 (Ad2), 5 (Ad5) or 35 (Ad35), or a chimeric adenovirus backbone comprising a combination of a portion of adenovirus serotype 2 (Ad2) or 5 (Ad5) with a portion of adenovirus serotype 35 (Ad35).

In one embodiment, the adenoviral vector of the invention is replication incompetent. Replication incompetent vectors traditionally lack one or more genes essential for replication. A replication incompetent vector does not replicate, or does so at very low levels, in the target cell. In one embodiment, a replication defective vector has at least one coding region in E1a, E1b, E2a, E2b or E4 inactivated, usually by deleting or mutating, part or all of the coding region. Methods for propagating these vectors are well known in the art. These replication incompetent viruses are propagated on cells that complement the essential gene(s) which are lacking. Replication incompetent adenoviral vectors have been used extensively to transduce cells in vitro and in vivo and express various transgenes.

Replication-defective Ad virions encapsulating the recombinant Ad vectors of the instant invention are made by standard techniques known in the art using Ad packaging cells and packaging technology. Examples of these methods may be found, for example, in U.S. Pat. No. 5,872,005, incorporated herein by reference in its entirety. In making an Ad vector according to the present invention, a multivalent soluble receptor protein-encoding sequence is inserted into adenovirus in the deleted E1A, E1B or E3 region of the virus genome. Preferred adenoviral vectors for use in practicing the invention do not express one or more wild-type Ad gene products, e.g., E1a, E1b, E2, E3, E4. Preferred embodiments are virions that are typically used together with packaging cell lines that complement the functions of E1, E2A, E4 and optionally the E3 gene regions. See, e.g. U.S. Pat. Nos. 5,872,005, 5,994,106, 6,133,028 and 6,127,175, expressly incorporated by reference herein in their entirety. Adenovirus vectors are purified and formulated using standard techniques known in the art.

In one embodiment, the adenoviral vector is replication-competent or replication conditional. Such vectors are able to replicate in a target cell. Replication competent viruses include wild-type viruses and viruses engineered to replicate in target cells. These include vectors designed to replicate specifically or preferentially in one type of target cell as compared to another. The target cell can be of a certain cell type, tissue type or have a certain cell status.

The DNA and protein sequences of Adenovirus serotypes 2 and 5 can be found in GenBank under accession number NC_{—}001405 (Ad2) and AY339865 (Ad5), both of which are incorporated herein in their entirety. Along with the complete genome DNA sequence, the GenBank entries include useful details such as references, location of splicing signals, polyadenylation sites, TATA signals, introns, start and stop codons for each identified gene, protein sequence, cDNA for each gene, and a list of sequence variations that exist throughout the literature. Also, of special interest with regards to the present invention, the mRNA structures for each region can be deduced from the indicated splicing site and polyadenylation cleavage site for each gene or region and the reference list of relevant publications in these GenBank records.

By way of example, an adenoviral vector based on adenoviral serotype 5 can be packaged into viral particles with extra sequences totaling up to about 105% of the genome size, or approximately 1.8 kb larger than the native Ad5 genome, without requiring deletion of viral sequences. If non-essential sequences are removed from the adenovirus genome, an additional 4.6 kb of insert can be tolerated (i.e., for a total insertion capacity of about 6.4 kb).

The viral vectors of this invention can be prepared using recombinant techniques that are standard in the art. Methods of modifying replication-competent or replication-incompetent viral vectors are well known in the art and are described herein and in publications cited herein. Various methods for cloning transgenes and desired transcriptional elements into adenovirus are described herein and are standard and well know in the art. The transgene and desired transcriptional elements are cloned into various sites in the adenoviral vector genome, as described herein. For example, there are various plasmids in the art that contain the different portions of the adenovirus genome, including plasmids that contain the entire adenovirus genome. The construction of these plasmids is also well described in the art (e.g. US20030104625). Once a site is selected for transgene(s) insertion an appropriate plasmid can be used to perform the modifications. Then the modifications may be introduced into a full-length adenoviral vector genome by, for example homologous recombination or in vitro ligation. The homologous recombination may take place in a mammalian cell (e.g. PerC6) or in a bacterial cell (e.g. E. Coli, see WO9617070). Manipulation of the viral vector genome can alternatively or in addition include well known molecular biology methods including, but not limited to, polymerase chain reaction (PCR), PCR-SOEing, restriction digests. If homologous recombination is employed, the two plasmids should share at least about 500 bp of sequence overlap, although smaller regions of overlap will recombine, but usually with lower efficiencies. Each plasmid, as desired, may be independently manipulated, followed by cotransfection in a competent host, providing complementing genes as appropriate for propagation of the adenoviral vector. Plasmids are generally introduced into a suitable host cell (e.g. 293, PerC.6, Hela-S3 cells) using appropriate means of transduction, such as cationic liposomes or calcium phosphate. Alternatively, in vitro ligation of the right and left-hand portions of the adenovirus genome can also be used to construct recombinant adenovirus derivative containing all the replication-essential portions of adenovirus genome. Berkner et al. (1983) Nucleic Acid Research 11: 6003-6020; Bridge et al. (1989) J. Virol. 63: 631-638.

Methods of packaging polynucleotides into adenovirus particles are known in the art and are also described in PCT PCT/US98/04080. The preferred packaging cells are those that have been designed to limit homologous recombination that could lead to wildtype adenoviral particles. Cells that may be used to produce the adenoviral particles of the invention include the human embryonic kidney cell line 293 (Graham et al., J Gen. Virol. 36:59-72 (1977)), the human embryonic retinoblast cell line PER.C6 (U.S. Pat. Nos. 5,994,128 and 6,033,908; Fallaux et al., Hum. Gene Ther. 9: 1909-1917 (1998)), and the human cervical tumor-derived cell line HeLa-S3 (PCT Application NO. US 04/11855).

For convenience, plasmids are available that provide the necessary portions of adenovirus. Plasmid pXC.1 (McKinnon (1982) Gene 19:33-42) contains the wild-type left-hand end of Ad5. pBHG10 (Bett et al. (1994); Microbix Biosystems Inc., Toronto) provides the right-hand end of Ad5, with a deletion in E3. Deletions in E3 provide more room in the viral vector to insert heterologous sequences. The gene for E3 is located on the opposite strand from E4 (r-strand). pBHG11 provides an even larger E3 deletion, an additional 0.3 kb is deleted (Bett et al. (1994). Alternatively, the use of pBHGE3 (Microbix Biosystems, Inc.) provides the right hand end of Ad5, with a full-length of E3.

The invention further provides a recombinant adenovirus particle comprising a recombinant viral vector according to the invention. In one embodiment, a capsid protein of the adenovirus particle comprises a targeting ligand. In one embodiment, the capsid protein is a fiber protein or pIX. In one embodiment, the capsid protein is a fiber protein and the ligand is in the C terminus or HI loop of the fiber protein. The adenoviral vector particle may also include other mutations to the fiber protein. In one embodiment, the ligand is added to the carboxyl end of the adenovirus fiber protein. In an additional embodiment, the virus is targeted by replacing the a portion of the fiber knob with a portions of a fiber knob from another adenovirus serotype. Examples of these mutations include, but are not limited to those described in U.S. application Ser. No. 10/403,337; US Application Publication No. 20040002060; PCT Publication Nos. WO 98/07877; WO 99/39734; WO 00/67576; WO 01/92299; and U.S. Pat. Nos. 5,543,328; 5,731,190; 5,756,086; 5,770,442; 5,846,782; 5,962,311; 5,922,315; 6,057,155; 6,127,525; 6,153,435; 6,455,314; 6,555,368 and 6,683,170 and Wu et al. (J Virol. 2003 Jul. 1; 77(13):7225-7235). These include, but are not limited to, mutations that decrease binding of the viral vector particle to a particular cell type or more than one cell type, enhance the binding of the viral vector particle to a particular cell type or more than one cell type and/or reduce the immune response to the adenoviral vector particle in an animal.

The vectors of the invention may also include enhancers and coding sequences for signal peptides. The vector constructs may or may not include an intron. Thus it will be appreciated that vectors of the invention may include any of a number of transgenes, combinations of transgenes and transgene/regulatory element combinations.

Exemplary replication competent adenoviral vectors are described for example in WO95/19434, WO97/01358, WO98/39465, WO98/39467, WO98/39466, WO99/06576, WO98/39464, WO00/20041, WO00/15820, WO00/39319, WO01/72994, WO01/72341, WO01/73093, WO03078592, WO 04/009790, WO 04/042025, WO96/17053, WO99/25860, WO 02/067861, WO 02/068627, each of which is expressly incorporated by reference herein.

Transgenes

The vectors of the invention may, in addition to coding for angiogenesis inhibitors of the invention, may include one or more other transgenes. Also, vectors and/or multivalent soluble receptor proteins of the invention may be used in combination with vectors encoding other transgenes. In one embodiment, these transgenes may encode for a marker. In one embodiment, these transgenes may encode for a cytotoxic protein. These vectors encoding a cytotoxic protein may be used to eliminate certain cells in either an investigational setting or to achieve a therapeutic effect. For example, in certain instances, it may be desirable to enhance the degree of therapeutic efficacy by enhancing the rate of cytotoxic activity. This could be accomplished by coupling the cell-specific replicative cytotoxic activity with expression of, one or more metabolic enzymes such as HSV-tk, nitroreductase, cytochrome P450 or cytosine deaminase (CD) which render cells capable of metabolizing 5-fluorocytosine (5-FC) to the chemotherapeutic agent 5-fluorouracil (5-FU), carboxylesterase (CA), deoxycytidine kinase (dCK), purine nucleoside phosphorylase (PNP), carboxypeptidase G2 (CPG2; Niculescu-Duvaz et al. J Med Chem. 2004 May 6; 47(10):2651-2658), thymidine phosphorylase (TP), thymidine kinase (TK) or xanthine-guanine phosphoribosyl transferase (XGPRT). This type of transgene may also be used to confer a bystander effect.

Additional transgenes that may be introduced into a vector of the invention include a factor capable of initiating apoptosis, antisense or ribozymes, which among other capabilities may be directed to mRNAs encoding proteins essential for proliferation of the cells or a pathogen, such as structural proteins, transcription factors, polymerases, etc., viral or other pathogenic proteins, where the pathogen proliferates intracellularly, cytotoxic proteins, e.g., the chains of diphtheria, ricin, abrin, etc., genes that encode an engineered cytoplasmic variant of a nuclease (e.g., RNase A) or protease (e.g., trypsin, papain, proteinase K, carboxypeptidase, etc.), chemokines, such as MCP3 alpha or MIP-1, pore-forming proteins derived from viruses, bacteria, or mammalian cells, fusgenic genes, chemotherapy sensitizing genes and radiation sensitizing genes. Other genes of interest include cytokines, antigens, transmembrane proteins, and the like, such as IL-1, IL-2, IL-4, IL-5, IL-6, IL-10, IL-12, IL-18 or flt3, GM-CSF, G-CSF, M-CSF, IFN-α, -β, -γ, TNF-α, -β, TGF-a, —P, NGF, MDA-7 (Melanoma differentiation associated gene-7, mda-7/interleukin-24), and the like. Further examples include, proapoptotic genes such as Fas, Bax, Caspase, TRAIL, Fas ligands, nitric oxide synthase (NOS) and the like; fusion genes which can lead to cell fusion or facilitate cell fusion such as V22, VSV and the like; tumor suppressor gene such as p53, RB, p16, p17, W9 and the like; genes associated with the cell cycle and genes which encode anti-angiogenic proteins such as endostatin, angiostatin and the like.

Other opportunities for specific genetic modification include T cells, such as tumor infiltrating lymphocytes (TILs), where the TILs may be modified to enhance expansion, enhance cytotoxicity, reduce response to proliferation inhibitors, enhance expression of lymphokines, etc. One may also wish to enhance target cell vulnerability by providing for expression of specific surface membrane proteins, e.g., B7, SV40 T antigen mutants, etc.

Although any gene or coding sequence of relevance can be used in the practice of the invention, certain genes, or fragments thereof, are particularly suitable. For example, coding regions encoding immunogenic polypeptides, toxins, immunotoxins and cytokines are useful in the practice of the invention. These coding regions include those hereinabove and additional coding regions include those that encode the following: proteins that stimulate interactions with immune cells such as B7, CD28, MHC class I, MHC class II, TAPs, tumor-associated antigens such as immunogenic sequences from MART-1, gp 100(pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, β-catenin, MUM-1, CDK-4, caspase 8, KIA 0205, HLA-A2R1701, α-fetoprotein, telomerase catalytic protein, G-250, MUC-1, carcinoembryonic protein, p53, Her2/neu, triosephosphate isomerase, CDC-27, LDLR-FUT, telomerase reverse transcriptase, PSMA, cDNAs of antibodies that block inhibitory signals (CTLA4 blockade), chemokines (MIP1α, MIP3α, CCR7 ligand, and calreticulin), anti-angiogenic genes include, but are not limited to, genes that encode METH-I, METH-2, TrpRS fragments, proliferin-related protein, prolactin fragment, PEDF, vasostatin, various fragments of extracellular matrix proteins and growth factor/cytokine inhibitors, various fragments of extracellular matrix proteins which include, but are not limited to, angiostatin, endostatin, kininostatin, fibrinogen-E fragment, thrombospondin, tumstatin, canstatin, restin, growth factor/cytokine inhibitors which include, but are not limited to, VEGF/VEGFR antagonist, sFlt-1, sFlk, sNRP1, angiopoietin/tie antagonist, sTie-2, chemokines (IP-10, PF-4, Gro-beta, IFN-gamma (Mig), IFNα, FGF/FGFR antagonist (sFGFR), Ephrin/Eph antagonist (sEphB4 and sephrinB2), PDGF, TGFβ and IGF-1. Genes suitable for use in the practice of the invention can encode enzymes (such as, for example, urease, renin, thrombin, metalloproteases, nitric oxide synthase, superoxide dismutase, catalase and others known to those of skill in the art), enzyme inhibitors (such as, for example, alpha1-antitrypsin, antithrombin III, cellular or viral protease inhibitors, plasminogen activator inhibitor-1, tissue inhibitor of metalloproteases, etc.), the cystic fibrosis transmembrane conductance regulator (CFTR) protein, insulin, dystrophin, or a Major Histocompatibility Complex (MHC) antigen of class I or II. Also useful are genes encoding polypeptides that can modulate/regulate expression of corresponding genes, polypeptides capable of inhibiting a bacterial, parasitic or viral infection or its development (for example, antigenic polypeptides, antigenic epitopes, and transdominant protein variants inhibiting the action of a native protein by competition), apoptosis inducers or inhibitors (for example, Bax, Bc12, Bc1X and others known to those of skill in the art), cytostatic agents (e.g., p21, p16, Rb, etc.), apolipoproteins (e.g., ApoAI, ApoAIV, ApoE, etc.), oxygen radical scavengers, polypeptides having an anti-tumor effect, antibodies, toxins, immunotoxins, markers (e.g., beta-galactosidase, luciferase, etc.) or any other genes of interest that are recognized in the art as being useful for treatment or prevention of a clinical condition. Further transgenes include those coding for a polypeptide which inhibits cellular division or signal transduction, a tumor suppressor protein (such as, for example, p53, Rb, p73), a polypeptide which activates the host immune system, a tumor-associated antigen (e.g., MUC-1, BRCA-1, an HPV early or late antigen such as E6, E7, L1, L2, etc), optionally in combination with a cytokine.

The invention further comprises combinations of two or more transgenes with synergistic, complementary and/or nonoverlapping toxicities and methods of action. In summary, the present invention provides methods for inserting transgene coding regions in specific regions of the viral vector genome. The methods take advantage of known viral transcription elements and the mechanisms for expression of Ad genes, reduce the size of the DNA sequence for transgene expression that is inserted into the Ad genome, since no additional promoter is necessary and the regulation signals encompass a smaller size DNA fragment, provide flexibility in temporal regulation of the transgene (e.g. early versus late stage of infection; early versus intermediate stage of infection), and provide techniques to regulate the amount of transgene expressed. For example, a higher amount of transgene can be expressed by inserting the transgene into a transcript that is expressed normally at high levels and/or by operatively linking a high efficiency splice acceptor site to the transgene coding region. Expression levels are also affected by how close the regulating signals are to their consensus sequences; changes can be made to tailor expression as desired.

In designing the adenoviral vectors of the invention the biological activity of the transgene is considered, e.g. in some cases it is advantageous that the transgene be inserted in the vector such that the transgene is only or mostly expressed at the late stages of infection (after viral DNA replication). For example, the transgene may be inserted, in L3, as further described herein. For some transgenes, it may be preferred to express the transgene early in the viral life cycle. In such cases, the transgene may be inserted in any of the early regions (for example, E3) or into the upstream L1 region.

Introducing Vectors into Cells

The vector constructs of the invention comprising nucleotide sequences encoding multivalent soluble receptor proteins of the invention may be introduced into cells in vitro, ex vivo or in vivo for delivery of multivalent soluble receptor proteins to cells, e.g., somatic cells, or in the production of recombinant multivalent soluble receptor proteins of the invention by vector-transduced cells using standard methodology known in the art. Such techniques include transfection using calcium phosphate, micro-injection into cultured cells (Capecchi, Cell 22:479-488 [1980]), electroporation (Shigekawa et al., BioTechn., 6:742-751 [1988]), liposome-mediated gene transfer (Mannino et al., BioTechn., 6:682-690 [1988]), lipid-mediated transduction (Felgner et al., Proc. Natl. Acad. Sci. USA 84:7413-7417 [1987]), and nucleic acid delivery using high-velocity microprojectiles (Klein et al., Nature 327:70-73 [1987]).

Viral construct encoding multivalent soluble receptor proteins of the invention may be introduced into cells using standard infection techniques routinely employed by those of skill in the art.

For in vitro or ex vivo expression, any cell effective to express a functional multivalent soluble receptor protein may be employed. Numerous examples of cells and cell lines used for protein expression are known in the art. For example, prokaryotic cells and insect cells may be used for expression. In addition, eukaryotic microorganisms, such as yeast may be used. The expression of recombinant proteins in prokaryotic, insect and yeast systems are generally known in the art and may be adapted for antibody expression using the compositions and methods of the present invention.

Examples of cells useful for multivalent soluble receptor protein expression further include mammalian cells, such as fibroblast cells, cells from non-human mammals such as ovine, porcine, murine and bovine cells, insect cells and the like. Specific examples of mammalian cells include COS cells, VERO cells, HeLa cells, Chinese hamster ovary (CHO) cells, 293 cell, NSO cells, SP20 cells, 3T3 fibroblast cells, W138 cells, BHK cells, HEPG2 cells, DUX cells and MDCK cells.

Host cells are cultured in conventional nutrient media, modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium (MEM, Sigma), RPMI 1640 (Sigma), and Dulbecco's Modified Eagle's Medium (DMEM, Sigma) are typically suitable for culturing host cells. A given medium is generally supplemented as necessary with hormones and/or other growth factors (such as insulin, transferrin, or epidermal growth factor), DHFR, salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The appropriate culture conditions for a particular cell line, such as temperature, pH and the like, are generally known in the art, with suggested culture conditions for culture of numerous cell lines provided, for example, in the ATCC Catalogue available on line at <“http://www.atcc.org/Search catalogs/AllCollections.cfm”>.

A vector encoding a multivalent soluble receptor proteins of the invention may be administered in vivo via any of a number of routes (e.g., intradermally, intravenously, intratumorally, into the brain, intraportally, intraperitoneally, intramuscularly, into the bladder etc.), effective to deliver the vector in animal models or human subjects. Dependent upon the route of administration, the recombinant multivalent soluble receptor protein will elicit an effect locally or systemically. The use of a tissue specific promoter 5′ to the multivalent soluble receptor protein open reading frame(s) results in greater tissue specificity with respect to expression of a recombinant protein expressed under control of a non-tissue specific promoter.

A vector encoding a multivalent soluble receptor proteins of the invention may be administered in vivo via any of a number of routes (e.g., intradermally, intravenously, intratumorally, into the brain, intraportally, intraperitoneally, intramuscularly, into the bladder etc.), effective to deliver the vector in animal models or human subjects. Dependent upon the route of administration, the recombinant multivalent soluble receptor protein will elicit an effect locally or systemically. The use of a tissue specific promoter 5′ to the multivalent soluble receptor protein open reading frame(s) results in greater tissue specificity with respect to expression of a recombinant protein expressed under control of a non-tissue specific promoter.

For example, in vivo delivery of the a recombinant AAV vector encoding a multivalent soluble receptor protein of the invention may be targeted to a wide variety of organ types including, but not limited to brain, liver, blood vessels, muscle, heart, lung and skin. In vivo delivery of the recombinant AAV vector may also be targeted to a wide variety of cell types based on the serotype of the virus, the status of the cells, i.e. cancer cells may be targeted based on cell cycle, the hypoxic state of the cellular environment or other physiological status that deviates from the typical, or normal, physiological state of that same cell when in a non-cancerous (non-dividing or regulated dividing state under normal, physiological conditions). Examples of cell status associated promoters include the telomerase reverse transcriptase promoter (TERT) and the E2F promoter.

In the case of ex vivo gene transfer, the target cells are removed from the host and genetically modified in the laboratory using a recombinant vector encoding a multivalent soluble receptor protein according to the present invention and methods well known in the art.

The recombinant vectors of the invention can be administered using conventional modes of administration including but not limited to the modes described above and may be in a variety of formulations which include but are not limited to liquid solutions and suspensions, microvesicles, liposomes and injectable or infusible solutions. The preferred form depends upon the mode of administration and the therapeutic application.

As the experimental results provided herein show, there are many advantages to be realized in using the inventive multivalent soluble receptor proteins of the invention in protein production in vivo, such as the administration of a single vector for long-term and sustained multivalent soluble receptor protein expression in patients; in vivo expression of the multivalent soluble receptor protein.

Recombinant vector constructs encoding a multivalent soluble receptor protein of the present invention find further utility in the in vitro production of recombinant protein for use in therapy. Methods for recombinant protein production are well known in the art and may be utilized for expression of recombinant multivalent soluble receptor protein using the vector constructs described herein.

Compositions and Methods for Practicing the Invention

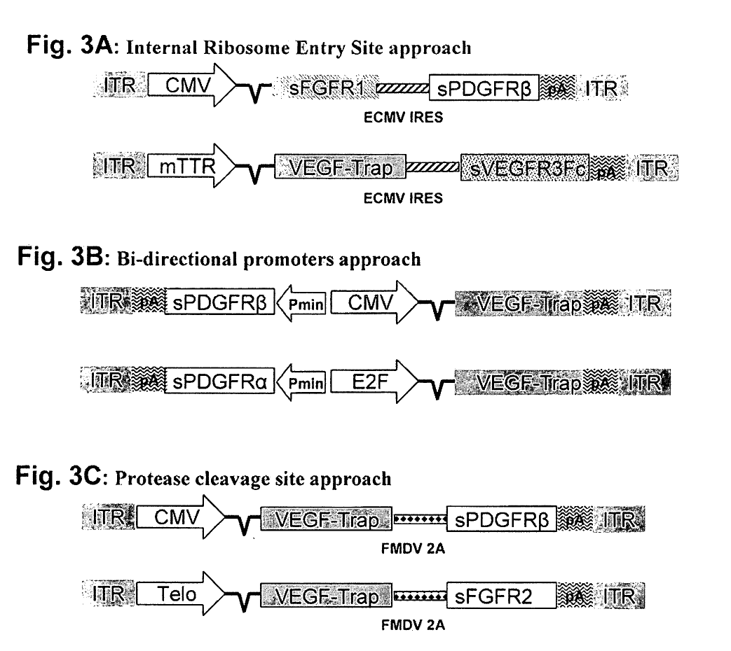

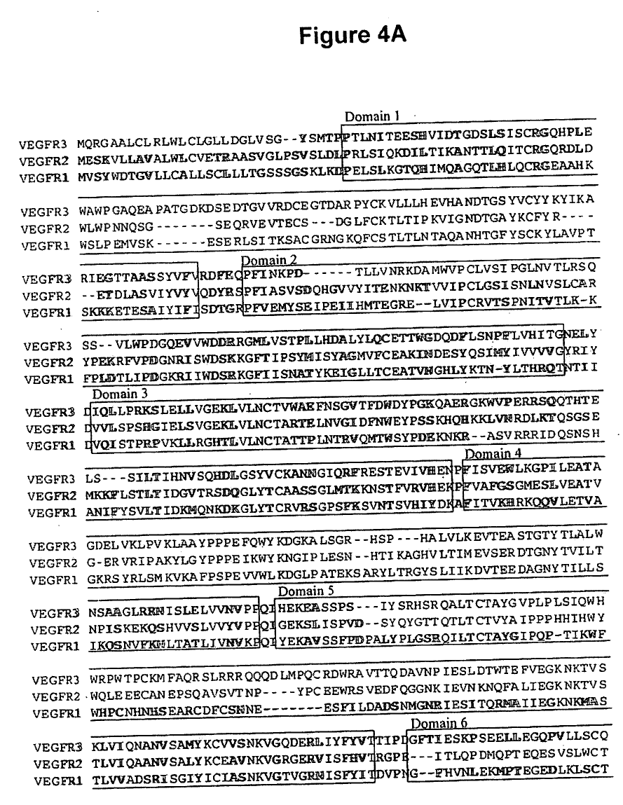

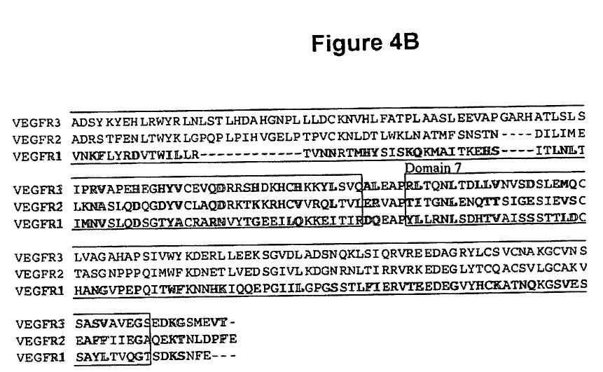

The invention provides single agents for inhibiting more than one angiogenic pathways, including nucleotide sequences and vectors for expression of multivalent soluble receptor fusion proteins (e.g., see FIGS. 3A-C) and multivalent soluble receptor proteins (e.g., see FIGS. 1A-C and 2A-H).

Nucleotide sequences that encode the multivalent soluble receptor proteins of the invention are constructed using standard recombinant DNA techniques. In most cases, these vectors are constructed so as to encode at least a portion of a receptor that is capable of binding an angiogenic factor without stimulating mitogenesis or angiogenesis. The portion of the receptor is generally part of the extracellular domain of a receptor that binds at least one angiogenic factor. For example, it may comprise Ig-like domains from one or multiple receptors that bind to an angiogenic factor.

In one embodiment, the polypeptides are multivalent soluble receptor proteins that bind at least two different angiogenic factors. In one embodiment, the two different angiogenic factors are from different families of angiogenic factors, e.g, a family of angiogenic factors selected from the group consisting of FGF, VEGF, PDGF, EGF, angiopoietins, Ephrins, placental growth factor, tumor growth factor alpha (TGFa), tumor growth factor beta (TGFb), tumor necrosis factor alpha (TNFa) and tumor necrosis factor beta (TNFb).

The invention further relates to a method of treating a subject having a neoplastic condition, comprising administering a therapeutically effective amount of a multivalent soluble receptor protein or vector encoding it to a subject, typically a patient with cancer. In a related embodiment, the multivalent soluble receptor proteins of the invention find utility in treatment of non neoplastic conditions by in vivo administration of a multivalent soluble receptor protein or vector encoding it to a subject. Alternatively, cells may be modified ex vivo and administered to a subject for treatment of a neoplastic or non neoplastic condition. Ex vivo modified cells are rendered proliferation incompetent prior to administration to a subject, typically by irradiation using techniques routinely employed by those of skill in the art.

Typically, the subject is a human patient. A therapeutically effective amount of a multivalent soluble receptor protein or vector encoding it is an amount effective at dosages and for a period of time necessary to achieve the desired result. This amount may vary according to various factors including but not limited to sex, age, weight of a subject, and the like.

An therapeutically effective amount of a vector encoding a of the invention is administered to a subject (e.g. a human) as a composition in a pharmaceutically acceptable excipient, including, but not limited to, saline solutions, suitable buffers, preservatives, stabilizers, and may be administered in conjunction with suitable agents such as antiemetics. An effective amount is an amount sufficient to effect beneficial or desired results, including clinical efficacy. An effective amount can be administered in one or more administrations. For purposes of this invention, an effective amount of vector is an amount that is sufficient to palliate, ameliorate, stabilize, reverse, slow or delay the progression of the disease state or alleviate symptoms of the disease. Some subject s are refractory to these treatments, and it is understood that the methods encompass administration to these subjects. The amount to be given will be determined by the condition of the individual, the extent of disease, the route of administration, how many doses will be administered, and the desired objective.

Delivery of vectors of the invention is generally accomplished by either site-specific injection or intravenous injection. Site-specific injections of vector may include, for example, injections into tumors, as well as intraperitoneal, intrapleural, intrathecal, intra-arterial, subcutaneous or topical application. These methods are easily accommodated in treatments using the combination of vectors and chemotherapeutic agents. The invention also contemplates the use of the vector to infect cells from the animal ex vivo. For example, cells are isolated from an animal. The isolated cells may contain a mixture of tumor cells and non-tumor cells. The cells are infected with a virus that is replication competent and the virus specifically replicates in tumor cells. Therefore, the tumor cells are eliminated and if desired the remaining non-tumor cells may be administered back to the same animal or if desired to a different animal.

The viral vectors may be delivered to the target cell in a variety of ways, including, but not limited to, liposomes, general transfection methods that are well known in the art (such as calcium phosphate precipitation or electroporation), direct injection, and intravenous infusion. The means of delivery will depend in large part on the particular vector (including its form) as well as the type and location of the target cells (i.e., whether the cells are in vitro or in vivo).

If used as a packaged virus, AAV vectors may be administered in an appropriate physiologically acceptable carrier at a dose of about 10⁴ to about 10¹⁴. If administered as a polynucleotide construct (i.e., not packaged as a virus) about 0.01 ug to about 1000 ug of an AAV vector can be administered. The exact dosage to be administered is dependent upon a variety of factors including the age, weight, and sex of the patient, and the size and severity of the condition being treated. The adenoviral vector(s) may be administered one or more times, depending upon the intended use and the immune response potential of the host, and may also be administered as multiple, simultaneous injections. If an immune response is undesirable, the immune response may be diminished by employing a variety of immunosuppressants, or by employing a technique such as an immunoadsorption procedure (e.g., immunoapheresis) that removes adenovirus antibody from the blood, so as to permit repetitive administration, without a strong immune response.

If packaged as another viral form, such as adenovirus or HSV, an amount to be administered is based on standard knowledge about that particular virus (which is readily obtainable from, for example, published literature) and can be determined empirically.

Combinations

Embodiments of the present invention include methods for the administration of combinations of a vector encoding a multivalent soluble receptor proteins of the present invention and/or a multivalent soluble receptor protein and a second anti-neoplastic therapy (e.g., a chemotherapeutic agent), which may include radiation, administration of an anti-neoplastic agent, etc., to an individual with neoplasia, as detailed in U.S. Application 2003/0068307. The vector and/or protein and anti-neoplastic agent may be administered simultaneously or sequentially, with various time intervals for sequential administration. In some embodiments, an effective amount of vector and/or multivalent soluble receptor protein and an effective amount of at least one anti-neoplastic agent are combined with a suitable excipient and/or buffer solutions and administered simultaneously from the same solution by any of the methods listed herein or those known in the art. This may be applicable when the anti-neoplastic agent does not compromise the viability and/or activity of the vector or protein itself.

Where more than one anti-neoplastic agent is administered, the agents may be administered together in the same composition; sequentially in any order; or, alternatively, administered simultaneously in different compositions. If the agents are administered sequentially, administration may further comprise a time delay. Sequential administration may be in any order, and accordingly encompasses the administration of an effective amount of a vector first, followed by the administration of an effective amount of the anti-neoplastic agent. The interval between administration of a vector which expresses a multivalent soluble receptor protein and/or the protein itself and chemotherapeutic agent may be in terms of at least (or, alternatively, less than) minutes, hours, or days. Sequential administration also encompasses administration of a chosen anti-neoplastic agent followed by the administration of the vector and/or protein. The interval between administration may be in terms of at least (or, alternatively, less than) minutes, hours, or days.

For therapeutic applications, the multivalent soluble receptor proteins of the present invention are administered to a mammal, preferably a human, in a pharmaceutically acceptable dosage form, including those that may be administered to a human intravenously as a bolus or by continuous infusion over a period of time, by intramuscular, intraperitoneal, intracerebrospinal, subcutaneous, intra-arterial, intrasynovial, intrathecal, oral, topical, or inhalation routes. The multivalent soluble receptor proteins of the present invention are also suitably administered by intratumoral, peritumoral, intralesional or perilesional routes.

In a further aspect of the invention, a pharmaceutical composition comprising a vector or chimeric multivalent soluble receptor protein of the invention and a pharmaceutically acceptable carrier is provided. Such compositions, which can comprise an effective amount of vector and/or chimeric multivalent soluble receptor protein in a pharmaceutically acceptable carrier, are suitable for local or systemic administration to individuals in unit dosage forms, sterile parenteral solutions or suspensions, sterile non-parenteral solutions or oral solutions or suspensions, oil in water or water in oil emulsions and the like. Formulations for parenteral and non-parenteral drug delivery are known in the art. Compositions also include lyophilized and/or reconstituted forms of the cancer-specific vector or particles of the invention. Acceptable pharmaceutical carriers are, for example, saline solution, protamine sulfate (Elkins-Sinn, Inc., Cherry Hill, N.J.), water, aqueous buffers, such as phosphate buffers and Tris buffers, or Polybrene (Sigma Chemical, St. Louis Mo.) and phosphate-buffered saline and sucrose. The selection of a suitable pharmaceutical carrier is deemed to be apparent to those skilled in the art from the teachings contained herein. These solutions are sterile and generally free of particulate matter other than the desired cancer-specific vector. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. Excipients that enhance uptake of the vector or chimeric multivalent soluble receptor protein by cells may be included.

For chimeric multivalent soluble receptor protein administration, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. No. 3,773,919, EP 58,481A, U.S. Pat. No. 3,887,699, EP 158,277A, Canadian Patent No. 1176565, U. Sidman et al., Biopolymers 22:547 (1983) and R. Langer et al., Chem. Tech. 12:98 (1982). The protein will usually be formulated in such vehicles at a concentration of about 0.01 mg/ml to 1000 mg/ml.

Optionally other ingredients may be added to pharmaceutical formulations such as antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

The vector or chimeric multivalent soluble receptor protein formulation to be used for therapeutic administration will in general be sterile. Sterility is readily accomplished through various methods known in the art, for example by filtration through sterile filtration membranes (e.g., 0.2 micron membranes). The vector or chimeric multivalent soluble receptor protein may be stored in lyophilized form or as an aqueous solution. The pH of vector or chimeric multivalent soluble receptor protein preparations typically will be about from 6 to 8, although higher or lower pH values may also be appropriate in certain instances.

For the prevention or treatment of disease, the appropriate dosage of a given vector or chimeric multivalent soluble receptor protein or will depend upon the type of disease to be treated, the severity and course of the disease, whether they are administered for preventative or therapeutic purposes, previous therapy, the patient's clinical history and response and in the case a human, the discretion of the attending physician. The vector or chimeric multivalent soluble receptor protein is suitably administered to the patient at one time or over a series of treatments.

Anti-neoplastic (chemotherapeutic) agents include those from each of the major classes of chemotherapeutics, including but not limited to: alkylating agents, alkaloids, antimetabolites, anti-tumor antibiotics, nitrosoureas, hormonal agonists/antagonists and analogs, immunomodulators, photosensitizers, enzymes and others. In some embodiments, the antineoplastic is an alkaloid, an antimetabolite, an antibiotic or an alkylating agent. In certain embodiments the antineoplastic agents include, for example, thiotepa, interferon alpha-2a, and the M-VAC combination (methotrexate-vinblastine, doxorubicin, cyclophosphamide). Preferred antineoplastic agents include, for example, 5-fluorouracil, cisplatin, 5-azacytidine, and gemcitabine. Particularly preferred embodiments include, but are not limited to, 5-fluorouracil, gemcitabine, doxorubicin, miroxantrone, mitomycin, dacarbazine, carmustine, vinblastine, lomustine, tamoxifen, docetaxel, paclitaxel or cisplatin. The specific choice of both the chemotherapeutic agent(s) is dependent upon, inter alia, the characteristics of the disease to be treated. These characteristics include, but are not limited to, location of the tumor, stage of the disease and the individual's response to previous treatments, if any.

There are a variety of delivery methods for the administration of antineoplastic agents, which are well known in the art, including oral and parenteral methods. There are a number of drawbacks to oral administration for a large number of antineoplastic agents, including low bioavailability, irritation of the digestive tract and the necessity of remembering to administer complicated combinations of drugs. The majority of parenteral administration of antineoplastic agents is intravenously, as intramuscular and subcutaneous injection often leads to irritation or damage to the tissue. Regional variations of parenteral injections include intra-arterial, intravesical, intra-tumor, intrathecal, intrapleural, intraperitoneal and intracavity injections.

Delivery methods for chemotherapeutic agents include intravenous, intraparenteral and intraperitoneal methods as well as oral administration. Intravenous methods also include delivery through a vein of the extremities as well as including more site specific delivery, such as an intravenous drip into the portal vein. Other intraparenteral methods of delivery include direct injections of an antineoplastic solution, for example, subcutaneously, intracavity or intra-tumor.

Assessment of the efficacy of a particular treatment regimen may be determined by any of the techniques employed by those of skill in the art to treat the subject condition, including diagnostic methods such as imaging techniques, analysis of serum tumor markers, biopsy, the presence, absence or amelioration of tumor associated symptoms. It will be understood that a given treatment regime may be modified, as appropriate, to maximize efficacy.

Utility

The multivalent soluble receptor proteins of the present invention find utility in the treatment of any and all cancers and related disorders. Exemplary cancers and related conditions that are amenable to treatment include cancers of the prostate, breast, lung, esophagus, colon, rectum, liver, urinary tract (e.g., bladder), kidney, liver, lung (e.g. non-small cell lung carcinoma), reproductive tract (e.g., ovary, cervix and endometrium), pancreas, gastrointestinal tract, stomach, thyroid, endocrine system, respiratory system, biliary tract, skin (e.g., melanoma), larynx, hematopoietic cancers of lymphoid or myeloid lineage, neurologic system, head and neck cancer, nasopharyngeal carcinoma (NPC), glioblastoma, teratocarcinoma, neuroblastoma, adenocarcinoma, cancers of mesenchymal origin such as a fibrosarcoma or rhabdomyosarcoma, soft tissue sarcoma and carcinoma, choriocarcinioma, hepatoblastoma, Karposi's sarcoma and Wilm's tumor.

Non-neoplastic conditions that are impacted by angiogenesis or lymph angiogenesis are also amenable to treatment using a chimeric multivalent soluble receptor fusion protein of the invention. For example, angiogenesis has been suggested to play a role in conditions such as rheumatoid arthritis, psoriasis, atherosclerosis, diabetic and other retinopathies, retrolentral fibroplasia, neovascular glaucoma, age-related macular degeneration, thyroid hyperplasias (including grave's disease), corneal and other tissue transplantation, chronic inflammation, lung inflammation, nephrotic syndrome, preclampasia, ascites, pericardial effusion (such as associated with pericarditis) and pleural effusion. As a result, these conditions may be treated using a vector or chimeric multivalent soluble receptor protein of the invention.

In another embodiment, the multivalent soluble receptor proteins that bind multiple angiogenesis promoting factors may be utilized to purify multiple angiogenic factors. For example, a multivalent soluble receptor protein that binds both VEGF and PDGF protein can be used to purify both of these proteins. The solution of VEGF and PDGF can then be used to study the process of angiogenesis or can be used to induce angiogenesis in a mammal including the induction of angiogenesis to treat a mammal. This eliminates the need to perform multiple purification processes to purify multiple angiogenic proteins. In this case, the term purification means that a significant amount of undesired protein is removed in the purification process and the resulting purified proteins are not necessarily 100% of the desired proteins. In one aspect, a significant amount of undesired protein is removed during the purification process. Protein purification procedures are known to those skilled in the art (see e.g., Scopes, Protein purification-principle and practice. Third Edition 1994).

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the preferred embodiments.

Materials and Methods

1. Characterization of Multivalent Soluble Receptor Proteins

Expression as well as the effectiveness of a given multivalent soluble receptor protein may be evaluated in vitro and in vivo using any of a number of methods known in the art.

For example, gene expression may be evaluated by measurement of the amount of multivalent soluble receptor protein or an IgG-like domain thereof following culture of cells that have been genetically modified to express a particular multivalent soluble receptor protein, e.g., by measurement of intracellular levels of expressed protein or by evaluation of the amount of expressed protein in the culture supernatant. Gene expression may also be evaluated in vivo, e.g., by determining the amount of a given multivalent soluble receptor protein in the serum of animals following administration of a viral vector encoding the protein. Such analyses may be carried out by a number of techniques routinely employed by those of skill in the art, including, but not limited to immunoassay, such as ELISA (as further described below), competitive immunoassay, radioimmunoassay, Western blot, indirect immunofluorescent assay and the like. The activity, expression and/or production of mRNA for a given multivalent soluble receptor fusion protein may also be determined by Northern blot and/or reverse transcriptase polymerase chain reaction (RT-PCR).

A. Detection by Immunoblotting and ELISA

Multivalent proteins are resolved using NuPage Bis-Tris gels and MOPS buffer by 4-12% SDS-PAGE (Invitrogen Life Technologies, Carlsbad, Calif.). Resolved proteins are transferred onto nitrocellulose for 1 hr in 20% methanol-containing transfer buffer (Invitrogen Life Technologies, Carlsbad, Calif.). Membranes are blocked for 1 hr in Tris-buffered saline (TBS) containing 5% BSA and 0.2% Tween-20 (ICN Pharmaceuticals, Inc., Costa Mesa, Calif.), and then probed with antiserum corresponding to the receptor construction (for VEGFR-3 biotinylated goat anti-VEGFR3 antiserum (R&D Systems, Minneapolis, Minn.)) for 1 hr. The blots are washed extensively with TBS-5% BSA, probed with HRP-conjugated-streptavidin (BD Pharmingen) for 1 hr, and subsequently visualized by enhanced chemiluminescence using the Supersignal substrate (Pierce, Rockford Ill.).

B. Quantification of Multivalent Soluble Receptor Proteins by IgG-Capture, IgG-Detect ELISA

Soluble VEGFR1-Fc is quantified using a commercially available sandwich ELISA kit (R&D Systems, Minneapolis, Minn.). Soluble VEGFR1/R2 is quantified using a sandwich ELISA technique using paired antibodies to human IgG1-Fc. Briefly, 96-well Immulon-4 microtiter plates (VWR, Willard, Ohio) are coated with goat anti-human IgG-Fc polyclonal antibody (Sigma Chemical Co., St. Louis, Mo.) in 0.1M carbonate pH 9.6 buffer and incubated overnight at 4° C. The plates are washed with PBS-0.05% Tween-20, and blocked with 2% non-fat milk diluent in borate buffer (KPL, Gaithersburg, Md.). Protein-G purified sVEGFR1/R2 protein from plasmid transfected HEK 293 cells is used for standard curves after serial dilutions using a 1% BSA diluent blocking solution (KPL, Gaithersburg, Md.). Diluted samples and the standard are incubated in the wells for 2 hr, washed extensively, and then incubated with 500 ng/ml HRP-conjugated anti-human IgG-Fc antibody (Bethyl Laboratories, Montgomery, Tex.) for 1 hr. After extensive washing, the samples are detected using ABTS peroxidase detection substrate at 450 nm optical density.

C. Evaluation Of Receptor Tyrosine Kinase (RTK) Blockade By Multivalent Soluble Receptor Proteins

Evaluation of Anti-Angiogenic Factors

The effectiveness of a given multivalent soluble receptor fusion protein in inhibiting the activity of associated factors may be evaluated in vitro using any of a number of methods known in the art. Exemplary in vitro angiogenesis assays include, but are not limited to, an endothelial cell migration assay, a Matrigel tube formation assay, endothelial and tumor cell proliferation assays, apoptosis assays and aortic ring assays.

In Vitro Assays

The rate of endothelial cell migration is evaluated using human umbilical vein endothelial cells (HUVEC) in a modified Boyden chamber assay (Clyman et al., 1994, Cell Adhes Commun. 1(4):333-42 and Lin, P et al., 1998, Cell Growth Differ. 9(1):49-58). A matrigel tube formation assay is used to demonstrate differentiation of endothelial cells. In carrying out the assay, endothelial cells are layered on top of an extracellular matrix (Matrigel), which allows them to differentiate into tube-like structures. Angiostatin, either in the form of fusion protein or protease treated plasminogen, has been shown to inhibit the proliferation of endothelial cells, migration of endothelial cells, inhibition of Matrigel tube formation and an induction of apoptosis of endothelial cells (O'Reily et al., Cell. 1994, 79(2):315-28 and Lucas et al., 1998, Blood 92(12):4730-41). Endothelial and tumor cell proliferation assays may be used to demonstrate the inhibitory effects of vector produced multivalent soluble receptor proteins on cell proliferation. An aortic ring assay has been used to demonstrate the inhibition of microvessel outgrowth of rat aorta rings by virally produced angiostatin and endostatin (Kruger, E. A. et al., 2000, Biophys. Res. Comm. 268, 183-191). Tumor cell apoptosis may also be evaluated as a further indicator of anti-angiogenic activity of multivalent soluble receptor proteins of the invention.

VEGF-A Inhibition Bioassay

HMVEC cells are seeded in 96-well flat-bottom plates at a density of 5×10³ cells/well and cultured overnight at 37° C. in a humidified incubator. The next day, the media is replaced with EBM-2 basal media (Cambrex, East Rutherford, N.J.) containing 5% FBS and incubated for 6 hr to deprive the cells of mitogenic growth factors. The cells are then stimulated with 20 ng/ml recombinant human VEGF (R&D Systems, Minneapolis, Minn.) in the presence, or absence, of increasing concentrations of a multivalent soluble receptor fusion protein. After 72 hr, cell proliferation is measured using a WST-8 tetrazolium salt-based Cell Counting Kit (Dojindo Laboratories, Gaithersburg, Md.) according to the manufacturer's specifications.

VEGF-C Inhibition Bioassay

A bioassay to investigate the blockade of VEGF-C biological activity is preformed as follows. BaF3/VEGFR3-EpoR cells (Makinen et al., Nat Med, 2001; 7(2): 199-205, 2001), a murine B-cell line stably expressing a multivalent soluble receptor fusion protein, e.g., a chimeric receptor comprised of the extracellular domain of VEGFR-3 and the intracellular domain of erythropoietin receptor (obtained from K. Alitalo, Univ. Helsinki, Finland) and maintained in Dulbeco's Modified Essential medium supplemented with 5% fetal bovine serum (GIBCO, Grand Island, N.Y.). BaF3/VEGFR3-EpoR cells are seeded at 1×10⁴ cells/well in 96-well titer plates and incubated overnight in 5% FBS-containing media. The following day, cells are stimulated with 100 ng/ml recombinant human VEGF-C (RnD Systems, Minneapolis, Minn.) in the presence of increasing concentrations of multivalent soluble receptor fusion protein. After 72 hrs, VEGF-C-mediated cell proliferation is measured by WST-8 tetrazolium salt using the Cell Counting Kit-8 (Dojindo Laboratories, Kumamato, Japan) according to the manufacturer's recommendations.

PDGF-BB and PDGF-AA Inhibition Bioassay

NIH 3T3 cells (ATCC, Manassas, Va.) are seeded at a density of 5×10³ cells/well on a 96-plate and cultured at 37° C. in a humidified incubator. Two days post-plating, the media is replaced with DMEM supplemented with 2% platelet-poor plasma (BioMedical Technologies, Stoughton, Mass.) containing and incubated for 6 hr to deprive the cells of mitogenic growth factors. The media is then removed and replaced with media containing 2% platelet-poor plasma and 10 ng/ml PDGF-BB (R&D Systems; for PDGF-BB stimulated bioassay) or 30 ng/ml pf PDGF-AA (R&D Systems; for PDGF-AAV stimulated bioassay) in the presence of increasing concentrations of multivalent soluble receptor fusion protein. After 48 hr, cell proliferation is measured using a WST-8 tetrazolium salt-based Cell Counting Kit (Dojindo Laboratories, Gaithersburg, Md.) according to the manufacturer's specifications.

HGF Proliferation Assay

HepG2 cells (ATCC, Manassas, Va.) are seeded at a density of 5×10³ cells/well in a 96 well plate in DMEM high (JRH Biosciences, Lanexa, Kans.) supplemented with 10% FBS. Twenty-four hours post-plating cells are starved for 6 hours in DMEM high without serum. Following serum starvation human recombinant HGF (R&D Systems, Minneapolis, Minn.) is added at a concentration of 10 ng/ml in the presence of increasing concentrations of multivalent soluble receptor fusion protein. 72 hours following HGF-addition, cell proliferation is measured using a WST-8 tetrazolium salt-based Cell Counting Kit (Dojindo Laboratories, Gaithersburg, Md.) according to the manufacturer's specifications.

bFGF Inhibition Bioassay

HMVEC cells are seeded in 96-well flat-bottom plates at a density of 5×10³ cells/well and cultured overnight at 37° C. in a humidified incubator. The next day, the media is replaced with EBM-2 basal media (Cambrex, East Rutherford, N.J.) for 4 hr to deprive the cells of mitogenic growth factors. The cells are then stimulated with 2 ng/ml recombinant human bFGF (R&D Systems, Minneapolis, Minn.) in the presence, or absence, of increasing concentrations of multivalent soluble receptor fusion protein. After 72 hr, cell proliferation is measured using a WST-8 tetrazolium salt-based Cell Counting Kit (Dojindo Laboratories, Gaithersburg, Md.) according to the manufacturer's specifications.

VEGF and bFGF Induced Endothelial Cell Migration Assay (Modified Boyden Chamber Migration Assay)

Briefly, a 24-well polycarbonate filter wells, (Costar Transwell with an 8 um pore size) are coated with 2% gelatin in PBS for 2-4 hours at room temperature in the cell culture hood, then subsequently incubated at 37 C for 1 h with DMEM containing 0.1% BSA. HUVEC cells are trypsinized, pelleted by centrifugation, washed and resuspended in fresh DMEM/BSA to a final concentration of 2×10⁶ cells/ml. Aliquots of cells 2×10⁵ cells are applied to the upper chamber of the filter wells. The filter inserts with cells are placed in wells of a 24-well culture plate containing either media alone as a control, or media plus human recombinant VEGF (for VEGF induced) or bFGF (for bFGF induced) at 10 ng/ml preincubated for 30 min with increasing concentrations of multivalent soluble receptor fusion protein. After a 6 hour incubation at 37 C, the cells that have migrated to the lower surface of the filter inserts are fixed with Diff-Quik (Dade International), fixed for 2 min; solution I for 2 min and solution II for 3 min. Filter inserts are examined under a microscope at 200× magnification.

Matrigel Tube Formation Assay—bFGF and VEGF

Matrigel (Beckton Dickinson) is coated onto 24-well cell culture plates on ice, and incubated at 37 C for 30 min. Conditioned medium from cells transduced with a vector construct which encodes a multivalent soluble receptor fusion protein is collected and assayed for production of anti-angiogenic activity. Conditioned medium is then titrated to contain 300 ng/ml of control protein and used to layer on top of the matrigel coated plates. 5×10⁵ HUVEC cells are added on top of the conditioned media. Plates are incubated for 12 hours at 37 C, and plates are scored by the total number of junctions formed by the endothelial cells from 5 fields and averaged under the microscope.

Aortic Ring Assay—bFGF Assay

12-well tissue culture plates are covered with Matrigel (Becton-Dickinson, Bedford, Mass.) and allowed to solidify for 1 hours at 37 C incubator. Thoracic aortas are excised from 4-6 week old male Sprague-Dawley rats and the fibroadipose tissue is removed. Aortas are sectioned into 1.2 mm long cross sections. Rinsed numerous times with EGM-2 (Clonetics Inc.), placed on Matrigel coated wells, and covered with additional Matrigel, then allowed to solidify at 37° C. for another hour. The rings are cultured overnight in 2 ml of EGM-2, the next day the media is removed, and the rings are cultured with bFGF and different concentrations of multivalent soluble receptor fusion protein for 4 days.

PDGFR-β Phospho-Tyrosine Kinase ELISA

U-87 MG human glioma cells are seeded at 5×10⁵ cells per well on 6-well plates in DMEM media (JRH Biosciences, Lanexa, Kans.) supplemented with 10% FBS. Forty-eight hours post-plating cells are starved in DMEM supplemented with 2% platelet-poor plasma for 24 hours. Following starvation cells are stimulated with 33 ng/ml of human PDGF-BB (R&D Systems, Minneapolis, Minn.) with or without multivalent soluble receptor fusion protein for 5 minutes in DMEM. Following stimulation cells are lysed and platelet-derived growth factor receptor β phosphorylation determined by phospho-specific ELISA according to manufacturer's instructions (R&D Systems, Minneapolis, Minn.).

D. In Vivo Tumor Models:

Exemplary in vivo angiogenesis models include, but are not limited to, in a B16 B1/6 mouse melanoma metastasis model; a B16F10-luc metastasis model with Xenogen Imaging (described below); a Lewis Lung Carcinoma (LLC) Xenograft Resection Model (O'Reilly et al, 1994, Cell. 79(2):315-28); a LLC-luc metastasis model/Xenogen Imaging; a LLC-luc SC resection model/Xenogen Imaging; a RIP-Tag pancreatic islet carcinoma transgenic model (Hanahan et al., Nature, 315(6015):115-122, 1985 and Bergers et al., Science, 284:808-811, 1999); an orthotopic breast cancer model MDA-231 (Hiraga T. et al., 2001, Cancer Res. 61(11):4418-24); a C6 glioma model (Griscelli F, et al., 1998, Proc Natl Acad Sci USA. 95(11):6367-72), a 4C8 glioma model (Weiner Nebr., et al. J Neuropathol Exp Neurol. 1999 January; 58(1):54-60), a U-251 MG glioma model (Ozawa T et al. In Vivo. 2002 January-February; 16(1):55-60) or a U-87 MG glioma model, an LnCP prostate cancer model (Horoszewicz J S et al., Cancer Res. 43(4):1809-18, 1983); and a PC-3 Xenograft pancreatic tumor model (Donaldson J T et al., 1990, Int J Cancer. 46(2):238-44).

Cells

The human U-87MG and rat C6 glioma tumor cells are purchased from ATCC (Manassas, Va.). The human U-251 MG glioblastoma cell line is obtained from the Department of Neurological Surgery Tissue Bank at the University of California, San Francisco. The 4C8 tumor cell line, derived from a spontaneously arising glioma in a transgenic MBP/c-neu mouse (Dyer and Philibotte 1995; Weiner et al. 1999), was kindly provided by Dr. C. A. Dyer (Children's Hospital of Philadelphia, Pa. All tumor cells are cultured in DMEM medium (JRH Biosciences, Lenexa, Kans.) supplemented with 10% irradiated FBS (JRH Biosciences, Lenexa, Kans.), 2 mM L-glutamine (JRH Biosciences, Lenexa, Kans.), 100 U/ml Penicillin and 100 ?g/ml streptomycin (Gibco BRL, Rockville, Md.).

Sub-Cutaneous Tumor Studies

Six- to eight-week-old female NCR nu/nude mice are obtained from Taconic (Germantown, N.Y.) and housed under SPF conditions. Animals are treated according to the ILAR Guide for the care and use of laboratory animals and all animal protocols are reviewed and approved by the Cell Genesys Institution Animal Care and Use Committee (ACUC). For systemic gene transfer studies, a vector construct (such as rAAV) which encodes a multivalent soluble receptor fusion protein is administered by a single tail-vein injection or intra-peritonial injection at varying dosage regimes. Mice are bled by alternate retro-orbital puncture on scheduled intervals to measure the serum level of circulating multivalent soluble receptor fusion protein by ELISA. For subcutaneous glioma tumor models C6 (2×10⁵ cells/site), 4C8 (2×10⁶ cells/site), U-251 MG (5×10⁶ cells/site) or U-87 MG (5×10⁶ cells/site) tumor cells are diluted in 100 ?l of sterile basal media and injected s.c. into the right dorsal flank. U-87 MG cells are pre-mixed with an equal volume of Matrigel (BD Biosciences, Mass.) prior to implantation. Mice are monitored daily for health and their tumors measured twice-weekly using digital calipers. Tumor volumes (as cubic millimeters) are calculated as volume=length×width²×0.5. Mice are euthanized as a “cancer death” when the s.c. tumor volume exceeds 1500 mm³ or when the tumors become excessively necrotic. Studies running longer than 80 days are actively terminated.

Orthotopic 4C8 Murine Glioblastoma Model

An orthotopic murine glioblastoma model in immunocompetent mice has been developed using a cell line, 4C8, derived from a spontaneous glioma-like tumor that arose in a transgenic mouse (Weiner N E, et al. J Neuropathol Exp Neurol. 1999 January; 58(1):54-60). Briefly, six week-old, male, B6D2F1 mice are obtained from Jackson Laboratories (Bar Harbor, Me.) and housed under SPF conditions. For tumor implantation, mice are anesthetized with pentobarbital and secured in a stereotactic head frame (David Kopf Instruments, Tujunga, Calif.). 4C8 cells (1×10⁶ cells in 5_{—}1) are injected into the left cerebral cortex at the level of the bregma, 2.0 mm from midline, at a depth of 2.0 mm through a 1 mm burr hole. Injections are done over 2 minutes using a 26 gauge Hamilton non-coring beveled needle (Hamilton Company, Reno, Nev.), and an UltraMicroPump II microinfuser (World Precision Instruments, Sarasota, Fla.). Seven days following 4C8 implantation, multivalent receptors are delivered by administration of: (a) a vector construct which encodes a multivalent soluble receptor fusion protein (e.g., rAAV) by a single tail-vein injection; or (b) intra-peritonial injection of a recombinant multivalent soluble receptor fusion protein at varying dosage regimes. For tumor size assessment, sequential MR images of 4C8 orthotopic tumors are acquired under general anesthesia using a Bruker Biospec DBX scanner (Bruker Medical, Billeria, Mass.) interfaced to an Oxford 7.0 Tesla/183 clear-bore magnet (Oxford Instruments, Oxford, UK). Tumors are localized as well demarcated areas of decreased signal intensity on both gradient and spin echo sequence images. Sequential MR images of brain with a 1.2 mm interslice distance are acquired and tumor area for each slice is calculated using NIH Image 1.62 software (NIH, Besthesda, Md.). Mice are euthanized and scored as a cancer death when they displayed significant adverse neurological systems as assessed by UC Davis ACUC institutional guidelines.

Orthotopic U-251 MG Glioblastoma Model

Four human glioblastoma were tested in an orthotopic rat model. The results indicated that U-251 MG and U-87 MG cells produce solid intracerebral tumors with a 100% tumor take rate, while SF-767 and SF-126 cells do not grow in the brains of athymic rats. The U-87 MG tumors were shown to grow faster than U-251 MG tumors, with both determined to be reproducible models for human glioblastoma (Ozawa T et al. In Vivo. 2002 January-February; 16(1):55-60). Briefly, six-week-old male athymic rats are purchased from Harlan (Indianapolis, Ind.) and housed under SPF conditions. U-251 MG tumor cells are implanted as previously described (Ozawa et al. 2002). 5×10⁶ U-251 cells are intra-cranially injected into the right caudate-putamen of the athymic rat using an implantable guide-screw system. Fifteen days post U-251 implantation, a 200_{—}1 Alzet osmotic minipump (Cupertino, Calif.) is inserted into a subcutaneous pocket in the midsacapular region on the back and a catheter is connected between the pump and a brain infusion cannula. Osmotic minipumps are loaded for administration of (a) a vector construct which encodes the multivalent soluble receptor fusion protein (e.g., rAAV); or (b) intra-peritonial injection of a recombinant multivalent soluble receptor fusion protein at varying dosage regimes over a 24-hour period (8_l/hr). Following agent delivery animals are monitored for survival scored as a cancer death when they displayed significant adverse neurological systems as assessed by UCSF ACUC institutional guidelines.

Immunohistochemistry

Tissues harvested from animals are fixed in 4% Paraformaldehyde, infiltrated with 30% sucrose, and frozen in OCT compound (Triangle Biomedical Sciences, Durham, N.C.). Cryostat sections are cut 25 microns (brain) or 5 microns (tumor) and mounted on Superfrost Plus slides (Fisher Scientific, Pittsburgh, Pa.). Specimens are rehydrated in TBS, permbeabilized with 0.1% TritonX-100 (Sigma) and incubated in 10% normal serum (Vector Labs, Burlingame, Calif.). Primary antibodies of interest are applied overnight at 4 degrees. The antibodies used are goat polyclonal anti-PECAM-1 (Santa Cruz Biotech, Santa Cruz, Calif.), rabbit polyclonal anti-human IgG (DAKO, Carpinteria, Calif.) mouse monoclonal PDGFRβ and Desmin (DAKO, Carpinteria, Calif.). The corresponding secondary antibodies, goat anti-rabbit Alexa 594 and rabbit anti-goat Alexa 594 (Molecular Probes, Eugene, Oreg.), are incubated for 30 minutes at room temperature. Slides are mounted in Vectashield Mounting Medium with DAPI (Vector Laboratories, Burlingame, Calif.) and analyzed by fluorescence microscopy using a Zeiss Axioplan (Germany) microscope equipped with a SPOT RT Slider digital camera (Diagnostic Instruments, Inc., Sterling Heights, Mich.). Quantification is done using Image Pro Plus (MediaCybernetics, Silver Springs, Md.) software.

E. In Vivo Metastasis Models

In Vitro Evaluation of Lymphangiogenesis And Lymphatic Metastasis

The effectiveness of a given vector encoding a multivalent soluble receptor fusion protein may be evaluated in vitro using any of a number of methods known in the art. Many in vitro assays to test for modulators of lymphangiogenesis are similar to those used to evaluate angiogenesis. For example, in vitro lymphangiogenesis assays may include, but are not limited to, lymphatic endothelial cell proliferation assays, lymphatic endothelial migration assays, and assays for the formation of lymphatic capillaries in response to pro-lymphangiogenic factors in vitro and ex vivo. Other assays may include testing the ability of the multivalent soluble receptor fusion protein to block the biochemical and biological activities of pro-lymphangiogenic growth factor signaling pathways in responsive cells. For example, the ability of sVEGFR3 to inhibit the lymphangiogenic growth factor, VEGF-C or VEGF-D, may be tested in responsive tissue culture cells which have been engineered to be mitogenic in response to VEGF-C stimulation. Blockage of vascular endothelial growth factor receptor 3 signaling has been shown to suppress tumor lymphangiogenesis and lymph node metastasis (He Y et al., J Natl Cancer Inst. 94(11):819-25, 2002).

In Vivo Evaluation of Lymphangiogenesis And Lymphatic Metastasis

The ability of sVEGFR3 to block lymphatic-mediated metastasis can be evaluated in animal models which have been developed for tumors that are dependent on lymphangiogenesis for their growth and spread. Exemplary models may include, but are not limited to, metastatic models of prostate, melanoma, breast, head & neck, and renal cell carcinomas. Tumor variant cell lines that preferentially metastasize to lymph nodes may be selected or tumor lines that highly express VEGF-C or VEGF-D may be used for development of animal tumor models for lymphatic metastases.

Cell Lines and Transfections

A human prostate cancer carcinoma cell line, PC-3, and a human melanoma cell line, A375, are purchased from ATCC (ATCC, Manassas, Va.). PC-3-mlg2 and A375-mln1 are sub-lines of PC-3 and A375 respectively, established by in vivo selection of lymph node metastases from PC-3 or A375 subcutaneous-tumor bearing mice (see Lin et al. 2005). PC-3-mlg2-VEGF-C is a sub-line of PC-3-mlg2, established by transduction with a lentiviral vector encoding human VEGF-C. The above tumor cell lines are maintained in RPMI-1640 (JRH Biosciences, Lanexa, Kans.) medium supplemented with 2 mM 1-glutamine, 100 U/ml penicillin, 100 ?g/ml streptomycin, and 10% fetal bovine serum (GIBCO, Grand Island, N.Y.) A human renal clear cell carcinoma cell line, Caki-2, i]l.ps purchased from ATCC and maintained in McCoy's 5A medium (JRH Biosciences, Lanexa, Kans.)) supplemented with 2 mM 1-glutamine, 100 U/ml penicillin, 100 ug/ml streptomycin, and 10% fetal bovine serum (ATCC, Manassas, Va.). All above tumor cell lines are transduced with a lentiviral vector expressing the firefly luciferase reporter gene.

Xenotransplantation and Metastasis Detection

All experiments performed on animals are in accordance with institutional guidelines. For selection of metastatic PC-3 variants, approximately 3×10⁶ luciferase-expressing PC-3 cells in 50 ?l of serum-free medium are implanted in the subcutaneous tissue of the dorsal flank of 7-9 week old female NCR nu/nude mice (one tumor per mouse). Tumors are measured with digital calipers, and the tumor volume (as cubic millimeters) are calculated as follows: volume=length×width²×0.5. Mice are euthanized after 6 weeks and the internal organs including the axillaries and inguinal lymph nodes from both sides are collected and analyzed by bioluminescence imaging. Briefly, the mice are administered with luciferin substrate (Xenogen Corp., Alameda, Calif.) at a dose of 1.5 mg/g mouse body weight by intraperitoneal injection. Fifteen minutes after substrate injection, the mice are euthanized; the lymph nodes are collected and placed in a Petri dish for bioluminescence imaging analysis. Lymph nodes with bioluminescence CCD counts above 1e⁵, detected by bioluminescence imaging analysis (Xenogen), are collected for establishment of primary culture. Briefly, the lymph nodes are minced and incubated with 0.5% trypsin at 37_C for 15 min. The reaction is stopped by adding 10% FBS-containing medium. The solution is collected and placed in a culture dish. Tumor cells are selected by repeated trypsinization every two days. After 5 passages, the tumor cells are harvested. Approximately 3×10⁶ cells in 50 ?l of serum-free medium are implanted in the subcutaneous tissue of the dorsal flank of female NCR nu/nude mice for outgrowth and further metastatic selection. PC-3-mlg2 tumor cells are established after two rounds of in vivo selection as described above. A375-mln2 tumor cells are selected following one round of selection using similar procedures as described above. Samples of tumors are snap-frozen in liquid nitrogen and stored at −70_C for RT-PCR and protein analysis, or fixed immediately in 4% paraformaldehyde for further histological analysis.

Evaluation of Lymph Node Metastasis

In efficacy studies, mice are administered multivalent receptors are delivered by administration of: (a) a vector construct which encodes the multivalent soluble receptor fusion protein (e.g., rAAV); or (b) injection of a recombinant multivalent soluble receptor fusion protein at varying dosage regimes. The animals are bled by alternate retro-orbital puncture on scheduled intervals thoughout the study to measure the serum levels (+/−sem) of multivalent proteins by ELISA. For PC-3 and A375 tumor models, animals are euthanized either five or three weeks post-tumor cell inoculation. For evaluation of lymphogenous metastasis, lymph nodes (including axillaries and inguinal nodes from both sides) are collected from each animal analyzed by bioluminescence imaging as described above. A set of six lymph nodes collected from a naïve mouse is used as negative control in each study. The metastases of each mouse are calculated based on total bioluminescence (CCD counts). In a separate study, 5×10⁶ Caki-2 tumor cells are administered ten days following multivalent protein administration. The lymph nodes (axillaries and inguinal nodes from both sides) are collected from each animal and the length and the width of lymph nodes are measured. The volumes (as cubic milliliters) are calculated as volume=(π/6)×(length×width)^{3/2}.

Quantitative Detection of Human Tumor Cell Metastasis

The detection of human tumor cells in mouse lymph nodes is based on the quantitative detection of human alu sequences present in mouse lymph nodes DNA extracts. Genomic DNA is extracted from harvested tissue using the Puregene DNA purification system (Centra Systems, Minneapolis, Minn.). To detect human cell in the mouse tissues, primers specific for human alu sequences are used to amplify the human alu repeats presented in genomic DNA that is extracted from the mouse lymph nodes. The real-time PCR used to amplify and detect alu sequences contained 30 ng of genomic DNA, 2 mm MgCl2, 0.4 ?M each primer, 200 ?M DNTP, 0.4 units of Platinum Taq polymerase (Invitrogen Corp, Carlsbad Calif.) and a 1:100,000 dilution of SYBR green dye) Molecular Probes, Eugene, Oreg.). Each PCR is performed in a final volume of 10 ul under 10 ul of mineral oil with the iCycler iQ (Bio-Rad lab, Hercules, Calif.) under the following conditions: polymerase activation at 95 C for 2 min followed by 30 cycles at 95 C for 30 s, 63 C for 30 s, and 72 C for 30 s. A quantitative measure of amplifiable mouse DNA is obtained through amplification of the mouse GAPDH genomic DNA sequence with mGAPDH primers using the same conditions described for alu. To approximate the actual number of tumor cells present in each tissue sample, a standard curve is generated through quantitative amplification of genomic DNA extracted from a serial dilution of human tumor cells mixed in tissue homogenates. By interpolating the alu signal from experimental samples with standard curve, the actual number of tumor cells/lymph node pool (six lymph nodes from each mouse) could be determined.

### EXAMPLES

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive. The following examples are offered by way of illustration and not by way of limitation.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Construction of sVEGFR-PDGFRb-Fc Fusion Encoding Plasmid

One method for constructing a recombinant plasmid termed pTR-CAG-VT.Pb.Fc that encodes the multivalent fusion protein sVEGFR-PDGFRb-Fc (FIG. 2A; SEQ ID NO:35) under the control of the CAG promoter is described in this example.

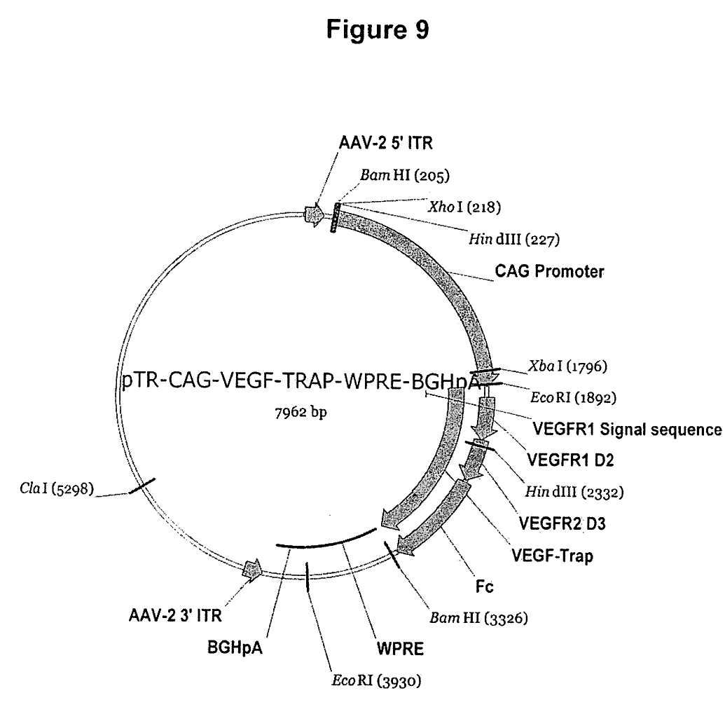

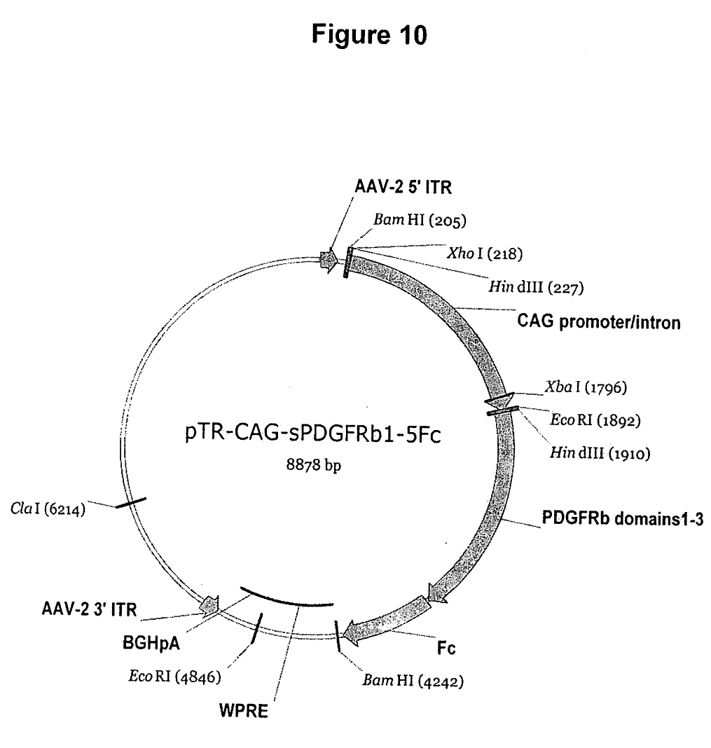

The plasmid is generated by cutting the plasmid pTR-CAG-sPDGFRb1-5Fc (FIG. 10; SEQ ID NO:39) with BglII, blunting the site with T4 DNA polymerase and then incubation with XbaI to extract a 8049 b.p. encoding PDGFRb Ig-like domains 1-5. This fragment is then ligated to the 801 b.p. XbaI-SmaI fragment of pTR-CAG-VEGF-TRAP-WPRE-BGHpA (FIG. 9; SEQ ID NO:38) creating pTR-CAG-VT.Pb.Fc. Recombinant structure is verified by restriction analysis and sequencing. SEQ ID NO:34 represents the composition of a sVEGFR-PDGFRb-IgG1 fusion protein.

### Example 2

### Construction of sPDGFRb-VEGFR-Fc Fusion Encoding Plasmid

One method for constructing a recombinant plasmid termed pTR-CAG-Pb.VT.Fc that encodes the multivalent fusion protein sPDGFRb-VEGFR-Fc (FIG. 2B; SEQ ID NO:35) under the control of the CAG promoter is described in this example. The plasmid is generated by taking the XbaI-ApaI fragment from the plasmid pTR-CAG-sPDGFRb1-5Fc (FIG. 10; SEQ ID NO:39) encoding PDGFRb Ig-like domains 1-5 and ligating into BspEI-XbaI sites in pTR-CAG-VEGF-TRAP-WPRE-BGHpA (FIG. 9; SEQ ID NO:38) using a linker (linker sequence 5′-CGGGCT-3′ (SEQ ID NO:40) and 5′-CCGGAGCCCGGGCC-3′ (SEQ ID NO:29) to create pTR-CAG-Pb.VT.Fc

### Example 3

### Construction of sVEGFR-Fc-PDGFRb Fusion Encoding Plasmid

One method for constructing a recombinant plasmid termed pTR-CAG-VT.Fc.Pb that encodes the multivalent fusion protein sVEGFR-Fc-PDGFRb (FIG. 2C; SEQ ID NO:36) under the control of the CAG promoter is described in this example. Initially the intermediate construct, pTR-CAG-VT.Fc.Pb.Fc is constructed by cloning the XbaI-NsiI fragment from pTR-CAG-VEGF-TRAP-WPRE-BGHpA (FIG. 9; SEQ ID NO:38) into the BglII-XbaI sites present in pTR-CAG-sPDGFRb1-5Fc (FIG. 10; SEQ ID NO:39) using a synthetic oligonucleotide linker (linker sequence forward 5′-TGAGGCTCTGCACAACCACTACACGCAGAAGAGCCTCTCCCTGTCTCCGGGTAAA CA-3′ (SEQ ID NO:30) and reverse 5′-GATCTGTTTACCCGGAGACAGGGAGAGGCTCTTCTGCGTGTAGTGGTTGTGCAGAGCCTCATGCA-3′ (SEQ ID NO:31). Following verification of pTR-CAG-VT.Fc.Pb.Fc sequence using restriction digest and sequencing, the secondary C-terminal IgG1 Fc region is removed by ligation of NotI-NsiI and NsiI-ApaI fragments from pTR-CAG-VT.Fc.Pb.Fc and synthetic linker (linker sequence forward 5′-TAACGCGTACCGGTGC-3′ (SEQ ID NO:32) and reverse 5′-GGCCGCACCGGTACGCGTTA-3′ (SEQ ID NO:33) following removal of the ApaI site by T4 DNA polymerase. The resulting plasmid structure of pTR-CAG-VT.Fc.Pb is verified by sequencing.

### Example 4

### Construction of sPDGFRb-Fc-VEGFR Fusion Encoding Plasmid

One method for constructing a recombinant plasmid termed pTR-CAG-Pb.Fc.VT that encodes the multivalent fusion protein sPDGFRb-Fc-VEGFR (FIG. 2D; SEQ ID NO:37) under the control of the CAG promoter is described in this example. Initially the intermediate construct pTR-CAG-Pb.Fc.VT.Fc is constructed by ligation of the XbaI-NsiI fragment from pTR-CAG-sPDGFRb1-5Fc (FIG. 10; SEQ ID NO: 39) with the BspEI-XbaI fragment from pTR-CAG-VEGF-TRAP-WPRE-BGHpA (FIG. 9; SEQ ID NO: 38) using a synthetic oligonucleotide linker (linker sequence forward 5′-TGAGGCTCTGCACAACCACTACACGCAGAAGAGCCTCTCCCTGTCTCCGGGTAAAT-3′ (SEQ ID NO:41) and reverse 5-CCGGATTTACCCGGAGACAGGGAGAGGCTCTTCTGCGTGTAGTGGTTGTGCAGAGCCTCATGCA-3′ (SEQ ID NO:47). Following verification of pTR-CAG-Pb.Fc.VT.Fc sequence using restriction digest and/or sequencing, the secondary C-terminal IgG1 Fc region is removed by ligation of the XbaI-BspEI fragment from pTR-CAG-Pb.Fc.VT.Fc to the BspEI-ApaI and NotI-XbaI fragments from pTR-CAG-VEGF-TRAP-WPRE-BGHpA (ApaI site removed by T4 DNA polymerase) and a synthetic linker (linker sequence forward 5′-TAACGCGTACCGGTGC-3′ (SEQ ID NO: 32) and reverse 5′-GGCCGCACCGGTACGCGTTA-3′ (SEQ ID NO: 33). The resulting plasmid structure of pTR-CAG-Pb.Fc.VT was verified by sequencing.

TABLE 1

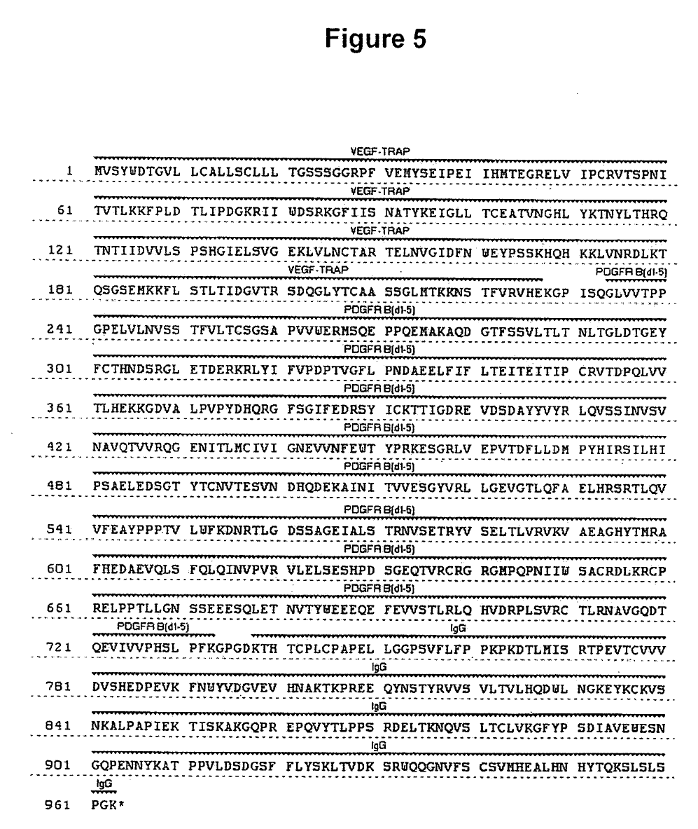

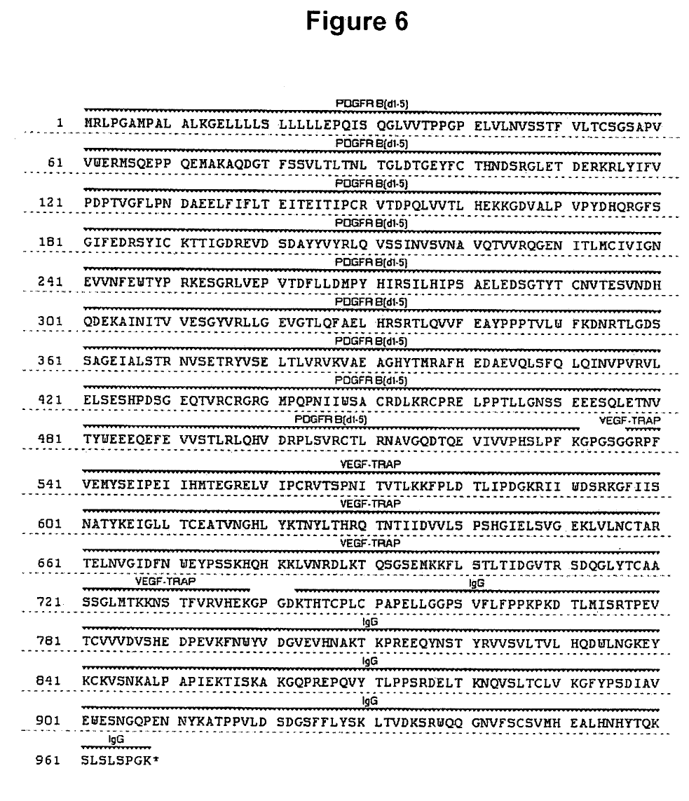

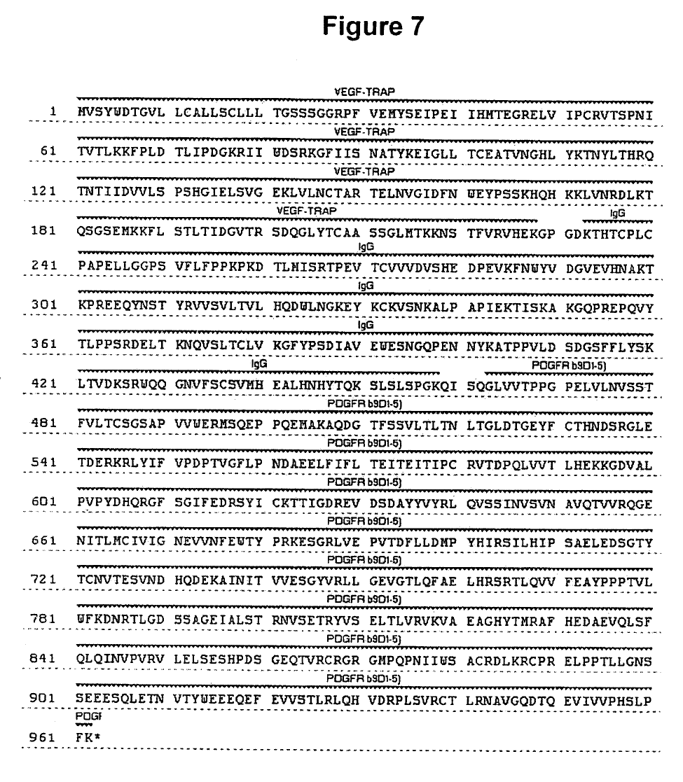

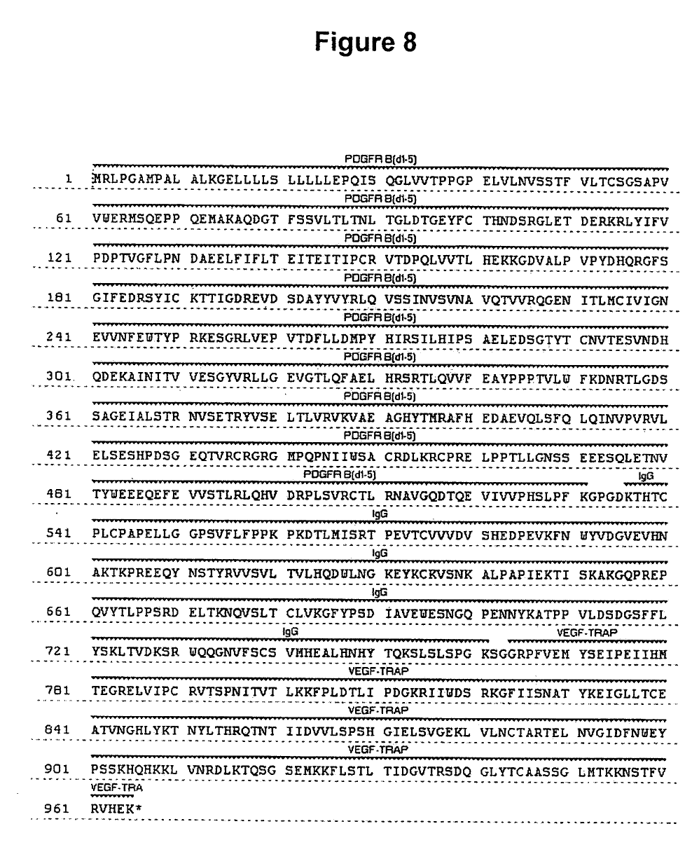

Table Of Sequences For Use In Practicing The
Invention

| | |
|---|---|
| SEQ | |
| ID | |
| NO | SUBJECT |

| | |
|---|---|
| 1 | VEGFR1 (FLT1) AMINO ACID SEQUENCE (1338 AMINO |
| | ACIDS) |
| 2 | VEGFR1 (FLT1) NUCLEOTIDE SEQUENCE-GENBANK |
| | ACCESSION No: NM_002019 (5777 NT)-CODING |
| | SEQUENCE IS NUCLEOTIDES 250-4266 |
| 3 | VEGFR1 (FLT1) AMINO ACID SEQUENCE |
| 4 | VEGFR2 (KDR) AMINO ACID SEQUENCE |
| 5 | VEGFR2 (KDR; A TYPE III RECEPTOR TYROSINE |
| | KINASE) NUCLEOTIDE SEQUENCE-GENBANK ACCESSION |
| | No: NM_002253 (5830 NT)-CODING SEQUENCE IS |
| | NUCLEOTIDES 304-4374 |
| 6 | VEGFR2 (KDR) AMINO ACIDS 1-327 |
| 7 | VEGFR3 (FLT4) AMINO ACID SEQUENCE |
| 8 | VEGFR3 (FLT4) NUCLEOTIDE SEQUENCE-GENBANK |
| | ACCESSION No: NM_182925 (4776 NT)-CODING |
| | SEQUENCE IS NUCLEOTIDES 21-4112 |
| 9 | VEGFR3 (FLT4) DOMAIN 1 AMINO ACIDS 30-132 |
| 10 | VEGFR3 (FLT4) DOMAIN 2 AMINO ACIDS 138-226 |
| 11 | VEGFR3 (FLT4) DOMAIN 3 AMINO ACIDS 232-329 |
| 12 | LINKER SEQUENCE: RDFEQ (BETWEEN DOMAINS 1 AND |
| | 2 OF VEGFR3) |
| 13 | LINKER SEQUENCE: NELYD (BETWEEN DOMAINS 2 AND |
| | 3 OF VEGFR3) |
| 14 | PLATELET-DERIVED GROWTH FACTOR RECEPTOR ALPHA |
| | (PDGF-ALPHA) AMINO ACID SEQUENCE |
| 15 | PDGF-ALPHA NUCLEOTIDE SEQUENCE-GENBANK |
| | ACCESSION No: NM_006206 (6405 NT)-CODING |
| | SEQUENCE IS NUCLEOTIDES 149-3418, SIGNAL |
| | SEQUENCE IS NUCLEOTIDES 149-217; THE MATURE |
| | PEPTIDE IS ENCODED BY NUCLEOTIDES 218-3415; THE |
| | POLYA SIGNAL IS NUCLEOTIDES 6366-6371 AND THE |
| | POLYA SITE IS ATNUCLEOTIDE 6391. |
| 16 | PLATELET-DERIVED GROWTH FACTOR RECEPTOR ALPHA: |
| | AMINO ACIDS 1-314 |
| 17 | PLATELET-DERIVED GROWTH FACTOR RECEPTOR BETA |
| | (PDGF-BETA) AMINO ACID SEQUENCE |
| 18 | PLATELET-DERIVED GROWTH FACTOR RECEPTOR BETA |
| | (PDGF-BETA) NUCLEOTIDE SEQUENCE-GENBANK |
| | ACCESSION No: NM_002609 (5598 NT)-CODING |
| | SEQUENCE IS NUCLEOTIDES 357-3677; SIGNAL |
| | SEQUENCE IS NUCLEOTIDES 357-452; THE MATURE |
| | PEPTIDE IS ENCODED BY NUCLEOTIDES 453-3674; |
| | THE POLYA SIGNAL IS NUCLEOTIDES 5574-5579 AND |
| | THE POLYA SITE IS AT NUCLEOTIDE 5598. |
| 19 | PLATELET-DERIVED GROWTH FACTOR RECEPTOR BETA: |
| | AMINO ACID SEQUENCE (LOKKERETAL. 1997) |
| 20 | FIBROBLAST GROWTH FACTOR RECEPTOR 1 (FGFR1) |
| | AMINO ACID SEQUENCE |
| 21 | FIBROBLAST GROWTH FACTOR RECEPTOR 1 (FGFR1) |
| | NUCLEOTIDE SEQUENCE-GENBANK ACCESSION No: |
| | NM_000604 (4049 NT)-CODING SEQUENCE IS |
| | NUCLEOTIDES 727-3195; SIGNAL SEQUENCE IS |
| | NUCLEOTIDES 727-789; THE MATURE PEPTIDE IS |
| | ENCODED BY NUCLEOTIDES 790-3192. |
| 22 | FIBROBLAST GROWTH FACTOR RECEPTOR 1: AMINO |
| | ACIDS 119-372 OF THE RECEPTOR (CHALLAIAH ET |
| | AL., J BIOL CHEM. 1999 DEC. 3; 274(49): |
| | 34785-94; OLSEN ET AL., PROC NATL ACAD SCI |
| | U.S.A. 2004; 101(4):935-40) |
| 23 | FIBROBLAST GROWTH FACTOR RECEPTOR 2 (FGFR1) |
| | AMINO ACID SEQUENCE |
| 24 | FIBROBLAST GROWTH FACTOR RECEPTOR 2: |
| | GENBANK ACCESSION No: NM_000141 (4587 NT)- |
| | CODING SEQUENCE IS NUCLEOTIDES 593-3058; |
| | SIGNAL SEQUENCE IS NUCLEOTIDES 593-655; |
| | THE MATURE PEPTIDE IS ENCODED BY NUCLEOTIDES |
| | 656-3055; |
| | THE POLYA SIGNAL IS NUCLEOTIDES 4553-4558 AND |
| | THE POLYA SITE IS AT NUCLEOTIDE 4571. |
| 25 | FIBROBLAST GROWTH FACTOR RECEPTOR 2: |
| | AMINO ACIDS 126-373 |
| 26 | HEPATOCYTE GROWTH FACTOR RECEPTOR AMINO ACID |
| | SEQUENCE |
| 27 | HEPATOCYTE GROWTH FACTOR RECEPTOR/C-MET |
| | RECEPTOR GENBANK ACCESSION No: LOCUS NM_000245 |
| | (6641 NT)-CODING SEQUENCE IS NUCLEOTIDES |
| | 188-4360; THE POLYA SIGNAL IS NUCLEOTIDES |
| | 6594-6599 AND POLYA SITES AT NUCLEOTIDES |
| | 6613, 6615 AND 6622. |
| 28 | HEPATOCYTE GROWTH FACTOR RECEPTOR/C-MET |
| | RECEPTOR: AMINO ACIDS 1-562 |
| 29 | LINKER NUCLEOTIDE SEQUENCE: CCGGAGCCCGGGCC |
| 30 | LINKER NUCLEOTIDE SEQUENCE:TGAGGCTCTGCACAACCAC |
| | TACACGCAGAAGAGCCTCTCCCTGTCTCCGGGTAAACA |
| 31 | LINKER NUCLEOTIDE SEQUENCE:CCGGATTTACCCGGAGACA |
| | GGGAGAGGCTCTTCTGCGTGTAGTGGTTGTGCAGAGCCTCATGCA |
| 32 | LINKER NUCLEOTIDE SEQUENCE: TAACGCGTACCGGTGC |
| 33 | LINKER NUCLEOTIDE SEQUENCE: GGCCGCACCGGTACGCGTTA |
| 34 | sVEGFR- PDGFR BETA DOMAINS 1-5-IGGFC |
| | NUCLEOTIDE SEQUENCE |
| 35 | SPDGFR BETA DOMAINS 1-5-VEGFR-IGGFC NUCLEOTIDE |
| | SEQUENCE |
| 36 | SVEGFR-IGGFC-PDGFR BETA DOMAINS 1-5 NUCLEOTIDE |
| | SEQUENCE |
| 37 | s PDGFR BETA DOMAINS 1-5-IGGFC-VEGFR NUCLEOTIDE |
| | SEQUENCE |
| 38 | pTR-CAG-VEGF-TRAP-WPRE-BGHpA NUCLEOTIDE |
| | SEQUENCE (7962 NTS) (FIG. 9) |
| 39 | PTR-CAG-sPDGFRB1-5Fc NUCLEOTIDE SEQUENCE |
| | (8878 NTS) (FIG. 10) |
| 40 | LINKER NUCLEOTIDE SEQUENCE: CGGGCT |
| 41 | LINKER NUCLEOTIDE SEQUENCE: |
| | TGAGGCTCTGCACAACCACTACACGCAGAAGAGCCTCTCCCTGTCTC |
| | CGGGTAAAT |
| 42 | TEK RECEPTOR TYROSINE KINASE AMINO ACID |
| | SEQUENCE |
| 43 | TEK RECEPTOR TYROSINE KINASE NUCLEOTIDE |
| | SEQUENCE; GENBANK ACCESSION No: NM_000459 (4138 |
| | NT)-CODING SEQUENCE IS NUCLEOTIDES 149-3523; |
| | SIGNAL SEQUENCE IS NUCLEOTIDES 149-202; THE |
| | MATURE PEPTIDE IS ENCODED BY NUCLEOTIDES |
| | 203-3520. |
| 44 | furin cleavage sites with the consensus |
| | sequence RXK(R)R |
| 45 | Furin cleavage consensus sequence RXR(K)R |
| 46 | Exemplary linker: Gly-Gly-Gly-Gly-Ser |
| 47 | synthetic oligonucleotide linker reverse |
| | 5-CCGGATFFFACCCGGAGACAGGGAGAGGCTCTTCTGCGTGTAGTG |
| | GTTGTGCAGAGCCTCATGCA-3′ |
| 48 | acid sequence of the extracellular domain |
| | of VEGFR3 |
| 49 | acid sequence of the extracellular domain of |
| | VEGFR2 |
| 50 | acid sequence of the extracellular domain of |
| | VEGFR1 |
| 51 | an annotated version of the amino acid |
| | sequence of the multivalent soluble receptor |
| | fusion proteins sVEGFR-PDGFR beta domains 1-5 |
| | IgGFc (FIG. 5) |
| 52 | an annotated version of the amino acid sequence |
| | of the multivalent fusion protein sPDGFR beta |
| | domains 1-5-VEGFR-IgGFc (FIG. 6) |
| 53 | an annotated version of the amino acid sequence |
| | of the multivalent fusion protein sVEGFR-IgGFc- |
| | sPDGFR beta domains 1-5 (FIG. 7) |
| 54 | an annotated version of the amino acid sequence |
| | of the multivalent fusion protein sPDGFR beta |
| | domains 1-5-IgGFc-VEGFR (FIG. 8) |

It is to be understood that while the invention has been described above in conjunction with preferred specific embodiments, the description and examples are intended to illustrate and not limit the scope of the invention, which is defined by the scope of the appended claims. All publications, sequences referred to in GenBank accession numbers, patents, and patent applications cited herein are hereby incorporated by reference for all purposes.

What is claimed is:

1. A nucleotide sequence encoding a multivalent soluble receptor protein comprising:
(a) the coding sequence for at least two domains selected from the group consisting of a PDGFR-alpha Ig-like domain, a PDGFR-beta Ig-like domain, a Fibroblast Growth Factor Receptor 1 (FGFR1) Ig-like domain, a Fibroblast Growth Factor Receptor 2 (FGFR2) Ig-like domain, a Hepatocyte Growth Factor Receptor (HGFR) SEMA domain-like domain; and
(b) the coding sequence for a heterologous multimerizing domain.

2. The nucleotide sequence of claim 1, wherein the multimerizing domain is an IgGFc domain.

3. The nucleotide sequence of claim 1, wherein the nucleotide sequence encodes at least one PDGFR-alpha Ig-like domain and at least one Fibroblast Growth Factor Receptor 1 (FGFR1) Ig-like domain.

4. The nucleotide sequence of claim 3, wherein the PDGFR-alpha Ig-like domain coding sequence comprises the sequence presented as SEQ ID NO:16.

5. The nucleotide sequence of claim 3, wherein the FGFR1 Ig-like domain coding sequence comprises the sequence presented as SEQ ID NO:22.

6. The nucleotide sequence of claim 1, wherein the nucleotide sequence encodes at least one PDGFR-alpha Ig-like domain and at least one Fibroblast Growth Factor Receptor 2 (FGFR2) Ig-like domain.

7. The nucleotide sequence of claim 6, wherein the PDGFR-alpha Ig-like domain coding sequence comprises the sequence presented as SEQ ID NO:16.

8. The nucleotide sequence of claim 6, wherein the FGFR2 Ig-like domain coding sequence comprises the sequence presented as SEQ ID NO:25.

9. The nucleotide sequence of claim 1, wherein the nucleotide sequence encodes at least one PDGFR-alpha Ig-like domain and the SEMA domain from Hepatocyte Growth Factor Receptor (HGFR)

10. The nucleotide sequence of claim 9, wherein the PDGFR-alpha Ig-like domain coding sequence comprises the sequence presented as SEQ ID NO:16.

11. The nucleotide sequence of claim 9, wherein the FGFR2 Ig-like domain coding sequence comprises the sequence presented as SEQ ID NO:25.

12. The nucleotide sequence of claim 1, wherein the nucleotide sequence encodes at least one PDGFR-beta Ig-like domain and at least one Fibroblast Growth Factor Receptor 1 (FGFR1) Ig-like domain.

13. The nucleotide sequence of claim 12, wherein the PDGFR-beta Ig-like domain coding sequence comprises the sequence presented as SEQ ID NO:19.

14. The nucleotide sequence of claim 12, wherein the FGFR1 Ig-like domain coding sequence comprises the sequence presented as SEQ ID NO:22.

15. The nucleotide sequence of claim 1, wherein the nucleotide sequence encodes at least one PDGFR-beta Ig-like domain and at least one Fibroblast Growth Factor Receptor 2 (FGFR2) Ig-like domain.

16. The nucleotide sequence of claim 15, wherein the PDGFR-beta Ig-like domain coding sequence comprises the sequence presented as SEQ ID NO:19.

17. The nucleotide sequence of claim 15, wherein the FGFR2 Ig-like domain coding sequence comprises the sequence presented as SEQ ID NO:25.

18. The nucleotide sequence of claim 1, wherein the nucleotide sequence encodes at least one PDGFR-beta Ig-like domain and the SEMA domain of Hepatocyte Growth Factor Receptor (HGFR)

19. The nucleotide sequence of claim 18, wherein the PDGFR-beta Ig-like domain coding sequence comprises the sequence presented as SEQ ID NO:19.

20. The nucleotide sequence of claim 18, wherein the HGFR SEMA domain coding sequence comprises the sequence presented as SEQ ID NO:28.

21. A nucleotide sequence encoding a multivalent soluble receptor protein comprising,
(a) the coding sequence for a Vascular Endothelial Growth Factor Receptor 1 (VEGFR1) Ig-like domain 2 and a Vascular Endothelial Growth Factor Receptor 2 (VEGFR2) Ig-like domain 3;
(b) the coding sequence for at least two additional domains selected from the group consisting of a PDGFR-alpha Ig-like domain, a PDGFR-beta Ig-like domain, a Fibroblast Growth Factor Receptor 1 (FGFR1) Ig-like domain, a Fibroblast Growth Factor Receptor 2 (FGFR2) Ig-like domain, a Hepatocyte Growth Factor Receptor (HGFR) SEMA domain; and
(c) the coding sequence for a multimerizing domain.

22. The nucleotide sequence of claim 21, wherein the multimerizing domain is an IgGFc domain.

23. The nucleotide sequence of claim 21, wherein the coding sequence encodes at least one PDGFR-alpha Ig-like domain.

24. The nucleotide sequence of claim 23, wherein the PDGFR-alpha Ig-like domain coding sequence comprises the sequence presented as SEQ ID NO:16.

25. The nucleotide sequence of claim 21, wherein the coding sequence encodes at least one PDGFR-beta Ig-like domain.

26. The nucleotide sequence of claim 25, wherein the PDGFR-beta Ig-like domain coding sequence comprises the sequence presented as SEQ ID NO:19.

27. The nucleotide sequence of claim 21, wherein the coding sequence encodes at least one FGFR1 Ig-like domain.

28. The nucleotide sequence of claim 27, wherein the FGFR1 Ig-like domain coding sequence comprises the sequence presented as SEQ ID NO:22.

29. The nucleotide sequence of claim 21, wherein the coding sequence encodes at least one FGFR2 Ig-like domain.

30. The nucleotide sequence of claim 29, wherein the FGFR2 Ig-like domain coding sequence comprises the sequence presented as SEQ ID NO:25.

31. The nucleotide sequence of claim 21, wherein the coding sequence encodes at least one HGFR SEMA domain.

32. The nucleotide sequence of claim 31, wherein the HGFR SEMA domain coding sequence comprises the sequence presented as SEQ ID NO:28.

33. A vector for expression of a multivalent soluble receptor protein, comprising the nucleotide sequence of claim 1.

34. A vector according to claim 33, wherein said vector is selected from the group consisting of an adeno associated virus (AAV) vector, a retroviral vector, a lentiviral vector, an adenovirus (Ad) vector, a simian virus 40 (SV 40) vector, a bovine papilloma virus vector, an Epstein Barr virus vector, a herpes virus vector, and a vaccinia virus vector.

35. The vector according to claim 34, wherein said vector is an AAV vector.

36. A host cell comprising the vector of claim 33.

37. A multivalent soluble receptor protein encoded by the vector of claim 33, wherein said expressed multivalent soluble receptor protein binds to more than one angiogenic factor.

38. A vector for expression of a multivalent soluble receptor protein, multivalent soluble receptor protein comprising the nucleotide sequence of claim 21.

39. A vector according to claim 38, wherein said vector is selected from the group consisting of an adeno associated virus (AAV) vector, a retroviral vector, a lentiviral vector, an adenovirus (Ad) vector, a simian virus 40 (SV 40) vector, a bovine papilloma virus vector, an Epstein Barr virus vector, a herpes virus vector, and a vaccinia virus vector.

40. The vector according to claim 39, wherein said vector is an AAV vector.

41. A host cell comprising the vector of claim 38.

42. A multivalent soluble receptor protein encoded by the vector of claim 38, wherein said expressed multivalent soluble receptor protein binds to more than one angiogenic factor.


SEQUENCE LISTING (54 sequences)

SEQ ID NO: 1 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 2 - DNA, 5777 bp, Homo sapiens
gcggacactcctctcggctcctccccggcagcggcggcggctcggagcgggctccggggctcgggtgcagcggccagcgggcctggcggcgaggattacccggggaagtggttgtctcctggctggagccgcgagacgggcgctcagggcgcggggccggcggcggcgaacgagaggacggactctggcggccgggtcgttggccgggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctccttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcccgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttctagaagcacatgtgtatttatacccccaggaaactagcttttgccagtattatgcatatataagtttacacctttatctttccatgggagccagctgctttttgtgatttttttaatagtgctttttttttttgactaacaagaatgtaactccagatagagaaatagtgacaagtgaagaacactactgctaaatcctcatgttactcagtgttagagaaatccttcctaaacccaatgacttccctgctccaacccccgccacctcagggcacgcaggaccagtttgattgaggagctgcactgatcacccaatgcatcacgtaccccactgggccagccctgcagcccaaaacccagggcaacaagcccgttagccccaggggatcactggctggcctgagcaacatctcgggagtcctctagcaggcctaagacatgtgaggaggaaaaggaaaaaaagcaaaaagcaagggagaaaagagaaaccgggagaaggcatgagaaagaatttgagacgcaccatgtgggcacggagggggacggggctcagcaatgccatttcagtggcttcccagctctgacccttctacatttgagggcccagccaggagcagatggacagcgatgaggggacattttctggattctgggaggcaagaaaaggacaaatatcttttttggaactaaagcaaattttagacctttacctatggaagtggttctatgtccattctcattcgtggcatgttttgatttgtagcactgagggtggcactcaactctgagcccatacttttggctcctctagtaagatgcactgaaaacttagccagagttaggttgtctccaggccatgatggccttacactgaaaatgtcacattctattttgggtattaatatatagtccagacacttaactcaatttcttggtattattctgttttgcacagttagttgtgaaagaaagctgagaagaatgaaaatgcagtcctgaggagagttttctccatatcaaaacgagggctgatggaggaaaaaggtcaataaggtcaagggaagaccccgtctctataccaaccaaaccaattcaccaacacagttgggacccaaaacacaggaagtcagtcacgtttccttttcatttaatggggattccactatctcacactaatctgaaaggatgtggaagagcattagctggcgcatattaagcactttaagctccttgagtaaaaaggtggtatgtaatttatgcaaggtatttctccagttgggactcaggatattagttaatgagccatcactagaagaaaagcccattttcaactgctttgaaacttgcctggggtctgagcatgatgggaatagggagacagggtaggaaagggcgcctactcttcagggtctaaagatcaagtgggccttggatcgctaagctggctctgtttgatgctatttatgcaagttagggtctatgtattta

SEQ ID NO: 3 - protein, 275 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKR

SEQ ID NO: 4 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 5 - DNA, 5830 bp, Homo sapiens
actgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctcttttttcattcatttaaaaagcattatcatgcccctgctgcgggtctcaccatgggtttagaacaaagagcttcaagcaatggccccatcctcaaagaagtagcagtacctggggagctgacacttctgtaaaactagaagataaaccaggcaacgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttagctgtctccttgccaagccccaggaagaaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttga

SEQ ID NO: 6 - protein, 327 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 7 - protein, 1363 aa, Homo sapiens
MQRGAALCLRLWLCLGLLDGLVSDYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGPYLCSVCRPKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEQRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRVRDGTRMRAPELATPAIRHIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 8 - DNA, 4776 bp, Homo sapiens
cccacgcgcagcggccggagatgcagcggggcgccgcgctgtgcctgcgactgtggctctgcctgggactcctggacggcctggtgagtgactactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctcaactgcaccgtgtgggctgagtttaactcaggtgtcacctttgactgggactacccagggaagcaggcagagcggggtaagtgggtgcccgagcgacgctcccaacagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggaccgtatctgtgcagcgtgtgcagacccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcagcgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcgcgtgagagacggcacaaggatgagggccccggagctggccactcccgccatacgccacatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcagctgtaaaggacctggccagaatgtggctgtgaccagggcacaccctgactcccaagggaggcggcggcggcctgagcggggggcccgaggaggccaggtgttttacaacagcgagtatggggagctgtcggagccaagcgaggaggaccactgctccccgtctgcccgcgtgactttcttcacagacaacagctactaagcagcatcggacaagacccccagcacttgggggttcaggcccggcagggcgggcagagggctggaggcccaggctgggaactcatctggttgaactctggtggcacaggagtgtcctcttccctctctgcagacttcccagctaggaagagcaggactccaggcccaaggctcccggaattccgtcaccacgactggccagggcacgctccagctgccccggcccctccccctgagattcagatgtcatttagttcagcatccgcaggtgctggtcccggggccagcacttccatgggaatgtctctttggcgacctcctttcatcacactgggtggtggcctggtccctgttttcccacgaggaatctgtgggtctgggagtcacacagtgttggaggttaaggcatacgagagcagaggtctcccaaacgccctttcctcctcaggcacacagctactctccccacgagggctggctggcctcacccacccctgcacagttgaagggaggggctgtgtttccatctcaaagaaggcatttgcagggtcctcttctgggcctgaccaaacagccaactagcccctggggtggccaccagtatgacagtattatacgctggcaacacagaggcagcccgcacacctgcgagtggcaaactgtcc

SEQ ID NO: 9 - protein, 103 aa, Homo sapiens
PTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFV

SEQ ID NO: 10 - protein, 89 aa, Homo sapiens
PFINKPDTLLVNRKDAMWVPCLVSIPGLNVTLRSQSSVLWPDGQEVVWDDRRGMLVSTPLLHDALYLQCETTWGDQDFLSNPFLVHITG

SEQ ID NO: 11 - protein, 98 aa, Homo sapiens
IQLLPRKSLELLVGEKLVLNCTVWAEFNSGVTFDWDYPGKQAERGKWVPERRSQQTHTELSSILTIHNVSQHDLGSYVCKANNGIQRFRESTEVIVHE

SEQ ID NO: 12 - protein, 5 aa, Artificial Sequence
  Description of Artificial Sequence Synthetic linker peptide
RDFEQ

SEQ ID NO: 13 - protein, 5 aa, Artificial Sequence
  Description of Artificial Sequence Synthetic linker peptide
NELYD

SEQ ID NO: 14 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 15 - DNA, 6405 bp, Homo sapiens
ggtttttgagcccattactgttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcgttctaaatatgaatgaatgggatattttgaaatgaactttgtcagtgttgcctcttgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttttcttcatgcctgatgaaagctttggcgaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggaattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgtgtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagttaaaaaaaaaaaaaaa

SEQ ID NO: 16 - protein, 314 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGF

SEQ ID NO: 17 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 18 - DNA, 5598 bp, Homo sapiens
ggcccctcagccctgctgcccagcacgagcctgtgctcgccctgcccaacgcagacagccagacccagggcggcccctctggcggctctgctcctcccgaaggatgcttggggagtgaggcgaagctgggcgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcactttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagccccaagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatggtaccccaagaaggatgtgagaggtggccgcttggagtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcac

SEQ ID NO: 19 - protein, 334 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSR

SEQ ID NO: 20 - protein, 822 aa, Homo sapiens [sequence omitted]

SEQ ID NO: 21 - DNA, 4049 bp, Homo sapiens
cctcttgcggccacaggcgcggcgtcctcggcggcgggcggcagctagcgggagccgggacgccggtgcagccgcagcgcgcggaggaacccgggtgtgccgggagctgggcggccacgtccggacgggaccgagacccctcgtagcgcattgcggcgacctcgccttccccggccgcgagcgcgccgctgcttgaaaagccgcggaacccaaggacttttctccggtccgagctcggggcgccccgcaggcgcacggtacccgtgctgcagtcgggcacgccgcggcgccgggggcctccgcagggcgatggagccggtctgcaaggaaagtgaggcgccgccgctgcgttctggaggaggggggcacaaggtctggagaccccgggtggcggacgggagccctccccccgccccgcctccggggcaccagctccggctccattgttcccgcccgggctggaggcgccgagcaccgagcgccgccgggagtcgagcgccggccgcggagctcttgcgaccccgccaggacccgaacagagcccgggggcggcgggccggagccggggacgcgggcacacgcccgctcgcacaagccacggcggactctcccgaggcggaacctccacgccgagcgagggtcagtttgaaaaggaggatcgagctcactgtggagtatccatggagatgtggagccttgtcaccaacctctaactgcagaactgggatgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctgactgccacccacacgccctccccagactccaccgtcagctgtaaccctcacccacagcccctgctgggcccaccacctgtccgtccctgtcccctttcctgctggcaggagccggctgcctaccaggggccttcctgtgtggcctgccttcaccccactcagctcacctctccctccacctcctctccacctgctggtgagaggtgcaaagaggcagatctttgctgccagccacttcatcccctcccagatgttggaccaacacccctccctgccaccaggcactgcctggagggcagggagtgggagccaatgaacaggcatgcaagtgagagcttcctgagctttctcctgtcggtttggtctgttttgccttcacccataagcccctcgcactctggtggcaggtgccttgtcctcagggctacagcagtagggaggtcagtgcttcgtgcctcgattgaaggtgacctctgccccagataggtggtgccagtggcttattaattccgatactagtttgctttgctgaccaaatgcctggtaccagaggatggtgaggcgaaggccaggttgggggcagtgttgtggccctggggcccagccccaaactgggggctctgtatatagctatgaagaaaacacaaagtgtataaatctgagtatatatttacatgtctttttaaaagggtcgttaccagagatttacccatcgggtaagatgctcctggtggctgggaggcatcagttgctatatattaaaaacaaaaaagaaaaaaaaggaaaatgtttttaaaaaggtcatatattttttgctacttttgctgttttatttttttaaattatgttctaaac

SEQ ID NO: 22 - protein, 253 aa, Homo sapiens
DALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSP

SEQ ID NO: 23 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 24 - DNA, 4587 bp, Homo sapiens
gagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccacaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgattctcttcagtgaattttgataatggcttccagactctttgcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttaaaaaaaaaaaaaaaa

SEQ ID NO: 25 - protein, 248 aa, Homo sapiens
DAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASP

SEQ ID NO: 26 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 27 - DNA, 6641 bp, Homo sapiens
gccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 28 - protein, 562 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICL

SEQ ID NO: 29 - DNA, 14 bp, Artificial Sequence
  Description of Artificial Sequence Synthetic linker sequence
ccggagcccgggcc

SEQ ID NO: 30 - DNA, 57 bp, Artificial Sequence
  Description of Artificial Sequence Synthetic linker sequence
tgaggctctgcacaaccactacacgcagaagagcctctccctgtctccgggtaaaca

SEQ ID NO: 31 - DNA, 65 bp, Artificial Sequence
  Description of Artificial Sequence Synthetic linker sequence
gatctgtttacccggagacagggagaggctcttctgcgtgtagtggttgtgcagagcctcatgca

SEQ ID NO: 32 - DNA, 16 bp, Artificial Sequence
  Description of Artificial Sequence Synthetic linker sequence
taacgcgtaccggtgc

SEQ ID NO: 33 - DNA, 20 bp, Artificial Sequence
  Description of Artificial Sequence Synthetic linker sequence
ggccgcaccggtacgcgtta

SEQ ID NO: 34 - DNA, 2892 bp, Homo sapiens
atggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaagggcccgatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaagggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 35 - DNA, 2907 bp, Homo sapiens
atgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaagggcccgggctccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaagggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 36 - DNA, 2889 bp, Homo sapiens
atggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaagggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaagtga

SEQ ID NO: 37 - DNA, 2898 bp, Homo sapiens
atgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaagggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaagtga

SEQ ID NO: 38 - DNA, 7962 bp, Homo sapiens
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggggctgcagcccgggggatccactagtactcgagctcaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctatggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaagggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagctagagcctgcggccgcaattgctagcgaatgcacttaaggatccgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtaccttgtggtgagatccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 39 - DNA, 8878 bp, Homo sapiens
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggggctgcagcccgggggatccactagtactcgagctcaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatcttaagcttatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaagggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagctagagcctgcggccgcaattgctagcgaatgcacttaaggatccgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtaccttgtggtgagatccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 40 - DNA, 6 bp, Artificial Sequence
  Description of Artificial Sequence Synthetic linker sequence
cgggct

SEQ ID NO: 41 - DNA, 56 bp, Artificial Sequence
  Description of Artificial Sequence Synthetic linker sequence
tgaggctctgcacaaccactacacgcagaagagcctctccctgtctccgggtaaat

SEQ ID NO: 42 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATIIQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 43 - DNA, 4138 bp, Homo sapiens
cttctgtgctgttccttcttgcctctaacttgtaaacaagacgtactaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcggctgtgatttcttggacaatattggatggctattctatttcttctattactatccgttacaaggttcaaggcaagaatgaagaccagcacgttgatgtgaagataaagaatgccaccatcattcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtacgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctaggacagaacatctgtataccctctgtttccctttcactggcatgggagacccttgacaactgctgagaaaacatgcctctgccaaaggatgtgatatataagtgtacatatgtgctggaattctaacaagtcataggttaatatttaagacactgaaaaatctaagtgatataaatcagattcttctctctcattttatccctcacctgtagcatgccagtcccgtttcatttagtcatgtgaccactctgtcttgtgtttccacagcctgcaagttcagtccaggatgctaacatctaaaaatagacttaaatctcattgcttacaagcctaagaatctttagagaagtatacataagtttaggataaaataatgggattttcttttcttttctctggtaatattgacttgtatattttaagaaataacagaaagcctgggtgacatttgggagacatgtgacatttatatattgaattaatatccctacatgtattgcacattgtaaaaagttttagttttgatgagttgtgagtttaccttgtatactgtaggcacactttgcactgatatatcatgagtgaataaatgtcttgcctactcaaaaaaaaaaaa

SEQ ID NO: 44 - protein, 5 aa, Artificial Sequence
  Description of Artificial Sequence Synthetic consensus sequence
RXKRR

SEQ ID NO: 45 - protein, 5 aa, Artificial Sequence
  Description of Artificial Sequence Synthetic consensus sequence
RXRKR

SEQ ID NO: 46 - protein, 5 aa, Artificial Sequence
  Description of Artificial Sequence Synthetic linker peptide
GGGGS

SEQ ID NO: 47 - DNA, 64 bp, Artificial Sequence
  Description of Artificial Sequence Synthetic linker sequence
ccggatttacccggagacagggagaggctcttctgcgtgtagtggttgtgcagagcctcatgca

SEQ ID NO: 48 - protein, 777 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYNTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCNVSSASVAVEGSEDKGSMEVT

SEQ ID NO: 49 - protein, 767 aa, Homo sapiens
MESKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLDPFE

SEQ ID NO: 50 - protein, 758 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNFE

SEQ ID NO: 51 - protein, 963 aa, Homo sapiens [sequence omitted]

SEQ ID NO: 52 - protein, 968 aa, Homo sapiens [sequence omitted]

SEQ ID NO: 53 - protein, 962 aa, Homo sapiens
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGKQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFK

SEQ ID NO: 54 - protein, 965 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKGPGDKTHTCPLCPAPELLGGPSVFLFPPKPKDTLMISRTPEVTCVVVDVSHEDPEVKRNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGKSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK